(19) United States
(12) Patent Application Publication
(10) Pub. No.: US 2011/0223671 A1
(43) Pub. Date: Sep. 15, 2011

(54) Methods for using positively and negatively selectable genes in a filamentous fungal cell

(75) Inventors: Wendy Yoder, Davis, CA (US); Jeffrey Shasky, Davis, CA (US)

(73) Assignee: Novozymes, Inc., Davis, CA (US)

(21) Appl. No.: 13/120,102

(22) PCT Filed: Sep. 30, 2009
(86) PCT No.: PCT/US09/59107

§ 371 Date: Jun. 1, 2011

Related U.S. Application Data
(60) Provisional application No. 61/101,276, filed on Sep. 30, 2008.

(51) Int. Cl. C12N 15/80; C12N 1/15; C12N 9/88; C07H 21/00; C12N 9/00

(52) U.S. Cl. 435/471; 435/320.1; 435/254.11; 435/232; 536/23.2; 435/183

(57) ABSTRACT

The present invention relates to methods for using positively and negatively selectable genes in a filamentous fungal cell to delete, disrupt, or insert a gene in a filamentous fungal cell.

### REFERENCE TO A SEQUENCE LISTING

This application contains a Sequence Listing in computer readable form. The computer readable form is incorporated herein by reference.

### BACKGROUND OF THE INVENTION

1. Field of the Invention

The present invention relates to methods for using positively and negatively selectable genes in a filamentous fungal cell.

2. Description of the Related Art

Selectable marker genes expressing specific phenotypes are widely used in recombinant DNA technology as part of an expression vector for identifying and isolating host cells into which a gene has been introduced. The product of a selectable marker gene can provide for biocide or viral resistance, resistance to heavy metals and the like, or may confer prototrophy to auxotrophs. Positively selectable genes are used to identify and/or isolate cells that have retained introduced genes, while negatively selectable genes provide a means for eliminating cells that retain the introduced gene.

The phenotype endowed by a positively selectable marker (e.g., resistance to a specific antibiotic), and thus the presence of the selectable marker gene in the cell/host, may be undesirable depending on the ultimate application of the cell/host, e.g., hygromycin B resistance gene in a commercial production strain. For this reason bi-directionally selectable marker genes, such as the Aspergillus nidulans acetamidase (amdS) gene, represent an attractive alternative. The amdS gene is a dominant, bi-directional selectable marker, in that the gene is dominant in both positive and negative directions. The advantage of the amdS gene is that it can be deleted or cured easily from a host organism by virtue of dominant negative selection, which is achieved by plating cells onto growth media containing fluoroacetamide. Fluoroacetamide is metabolized by amdS-harboring cells to fluoroacetic acid, which is toxic to the cells. Only those cells having lost the amdS gene can grow under negative selection conditions. However, one major problem with the use of amdS as a selectable marker is that it is fairly widespread throughout the fungal kingdom and any active endogenous copies of the gene in the wild-type host strain must be inactivated or deleted prior to use of the amdS gene as a selectable marker. Relatively few other bi-directionally-selectable marker genes are available (e.g., pyrG, sC, niaD, and oliC), but they suffer from the disadvantage of requiring generation of auxotrophic mutants prior to their utilization, which may introduce unknown and undesirable mutations into the host genome, and these systems may not function in all fungi. For example, some Fusarium strains can metabolize 5-fluoroorotic acid, rendering pyrG ineffective as a bi-directionally selectable marker. Consequently, there is a need in the art for new methods for using positive and negative phenotypes in filamentous fungi.

U.S. Pat. No. 6,555,370 discloses the use of bi-functional selectable fusion genes.

There is also a need in the art to provide different methods for removing extraneous DNA, e.g., selectable marker, introduced into a genetically engineered filamentous fungus so the fungus contains only minimal traces to none of the DNA that was used in the generation of the recombinant strain. Any technology that provides for removal of such DNA is valuable to the art.

The present invention provides methods for using positively and negatively selectable genes in a filamentous fungal cell.

### SUMMARY OF THE INVENTION

The present invention relates to methods for deleting a gene or a portion thereof in the genome of a filamentous fungal cell, comprising:

(a) introducing into the filamentous fungal cell a nucleic acid construct comprising:
(i) a first polynucleotide comprising a dominant positively selectable marker coding sequence, which when expressed confers a dominant positively selectable phenotype on the filamentous fungal cell;
(ii) a second polynucleotide comprising a negatively selectable marker coding sequence, which when expressed confers a negatively selectable phenotype on the filamentous fungal cell;
(iii) a first repeat sequence located 5′ of the first and second polynucleotides and a second repeat sequence located 3′ of the first and second polynucleotides, wherein the first and second repeat sequences comprise identical sequences; and
(iv) a first flanking sequence located 5′ of components (i), (ii), and (iii) and a second flanking sequence located 3′ of the components (i), (ii), and (iii), wherein the first flanking sequence is identical to a first region of the genome of the filamentous fungal cell and the second flanking sequence is identical to a second region of the genome of the filamentous fungal cell, wherein (1) the first region is located 5′ of the gene or a portion thereof and the second region is located 3′ of the gene or a portion thereof of the filamentous fungal cell, (2) both of the first and second regions are located within the gene of the filamentous fungal cell, or (3) one of the first and second regions is located within the gene and the other of the first and second regions is located 5′ or 3′ of the gene of the filamentous fungal cell;

wherein the first and second flanking sequences undergo intermolecular homologous recombination with the first and second regions of the filamentous fungal cell, respectively, to delete and replace the gene or a portion thereof with the nucleic acid construct;

(b) selecting cells having a dominant positively selectable phenotype from step (a) by applying positive selection; and

(c) selecting cells having a negatively selectable phenotype from the selected cells having the dominant positively selectable phenotype of step (b) by applying negative selection to force the first and second repeat sequences to undergo intramolecular homologous recombination to delete the first and second polynucleotides.

The present invention also relates to methods for introducing a polynucleotide of interest into the genome of a filamentous fungal cell, comprising:

(a) introducing into the filamentous fungal cell a nucleic acid construct comprising:
(i) a first polynucleotide of interest;
(ii) a second polynucleotide comprising a dominant positively selectable marker coding sequence, which when expressed confers a dominant positively selectable phenotype on the filamentous fungal cell;
(iii) a third polynucleotide comprising a negatively selectable marker coding sequence, which when expressed confers a negatively selectable phenotype on the filamentous fungal cell;
(iv) a first repeat sequence located 5′ of the second and third polynucleotides and a second repeat sequence located 3′ of the second and third polynucleotides, wherein the first and second repeat sequences comprise identical sequences and the first polynucleotide of interest is located either 5′ of the first repeat or 3′ of the second repeat; and
(v) a first flanking sequence located 5′ of components (i), (ii), (iii), and (iv) and a second flanking sequence located 3′ of the components (i), (ii), (iii), and (iv), wherein the first flanking sequence is identical to a first region of the genome of the filamentous fungal cell and the second flanking sequence is identical to a second region of the genome of the filamentous fungal cell;

wherein the first and second flanking sequences undergo intermolecular homologous recombination with the first and second regions of the genome of the filamentous fungal cell, respectively, to introduce the nucleic acid construct into the genome of the filamentous fungal cell;

(b) selecting cells having a dominant positively selectable phenotype from step (a) by applying positive selection; and

(c) selecting and isolating a cell having a negatively selectable phenotype from the selected cells having the dominant positively selectable phenotype of step (b) by applying negative selection to force the first and second repeat sequences to undergo intramolecular homologous recombination to delete the second and third polynucleotides.

The present invention also relates to such nucleic acid constructs and vectors and filamentous fungal cells comprising such nucleic acid constructs.

### BRIEF DESCRIPTION OF THE FIGURES

FIG. 1 shows a restriction map of pJaL504-[Bam HI].

FIG. 2 shows a restriction map of pJaL504-[Bgl II].

FIG. 3 shows a restriction map of pJaL574.

FIG. 4 shows a restriction map of pWTY1449-02-01.

FIG. 5 shows a restriction map of pEJG61.

FIG. 6 shows a restriction map of pEmY21.

FIG. 7 shows a restriction map of pDM156.2.

FIG. 8 shows a restriction map of pEmY23.

FIG. 9 shows a restriction map of pWTY1470-19-07.

FIG. 10 shows a restriction map of pWTY1515-02-01.

FIG. 11 shows a restriction map of pJfyS1540-75-5.

FIG. 12 shows a restriction map of pJfyS1579-1-13.

FIG. 13 shows a restriction map of pJfyS1579-8-6.

FIG. 14 shows a restriction map of pJfyS1579-21-16.

FIG. 15 shows a restriction map of pAlLo1492-24.

FIG. 16 shows a restriction map of pJfyS1579-35-2.

FIG. 17 shows a restriction map of pJfyS1579-41-11.

FIG. 18 shows a restriction map of pJfyS1604-55-13.

FIG. 19 shows a restriction map of pJfyS1579-93-1.

FIG. 20 shows a restriction map of pJfyS1604-17-2.

FIG. 21 shows a restriction map of pEJG69.

FIG. 22 shows a restriction map of pEJG65.

FIG. 23 shows a restriction map of pMStr19.

FIG. 24 shows a restriction map of pEJG49.

FIG. 25 shows a restriction map of pEmY15.

FIG. 26 shows a restriction map of pEmY24.

FIG. 27 shows a restriction map of pDM257.

FIG. 28 shows a restriction map of pDM258.

FIG. 29 shows the relative lactose oxidase yields of transformants of a Fusarium venenatum amyA-deleted strain.

FIG. 30 shows the relative alpha-amylase activity of transformants of a Fusarium venenatum amyA-deleted strain.

FIG. 31 shows a restriction map of pJfyS1698-65-15.

FIG. 32 shows a restriction map of pJfyS1698-72-10.

FIG. 33 shows the relative alkaline protease activity of transformants of a Fusarium venenatum alpA-deleted strain.

FIG. 34 shows a restriction map of pJfyS1879-32-2.

FIG. 35 shows a restriction map of pJfyS111.

FIG. 36 shows a restriction map of pJfyS2010-13-5.

FIG. 37 shows a restriction map of pJfyS120.

### DEFINITIONS

Selectable marker: The term “selectable marker” is defined herein as a gene encoding a protein capable of conferring an antibiotic resistance phenotype, supplying an autotrophic requirement (for dominant positive selection), or activating a toxic metabolite (for negative selection).

Dominant positively selectable marker: The term “dominant positively selectable marker” is defined herein as a gene which, upon being transformed into a filamentous fungal cell, expresses a dominant phenotype permitting positive selection of transformants.

Dominant positively selectable phenotype: The term “dominant positively selectable phenotype” is defined herein as a phenotype permitting positive selection of transformants.

Negatively selectable marker: The term “negatively selectable marker” is defined herein as a gene which, upon being transformed into a filamentous fungal cell, expresses a phenotype permitting negative selection (i.e., elimination) of transformants.

Negatively selectable phenotype: The term “negatively selectable phenotype” is defined herein as a phenotype permitting negative selection (i.e., elimination) of transformants.

Gene: The term “gene” is defined herein as a region of DNA of the genome of a cell, which controls a discrete hereditary characteristic, usually corresponding to a single protein or RNA. Encompassed within the term “gene” is the entire functional unit including coding sequences, non-coding sequences, introns, promoter, and other regulatory sequences that encode proteins that alter expression.

Portion thereof: The term “portion thereof” is defined herein as a component of the entire functional unit of a gene such as an open reading frame (ORF), promoter, intronic sequence, and other regulatory sequences; or a part thereof.

Located 5′ or 3′ of the first and second polynucleotides: The terms “located 5′ of the first and second polynucleotides” and “located 3′ of the first and second polynucleotides” are defined herein as preferably within 1000 to 5000 bp, more preferably within 100 to 1000 bp, even more preferably within 10 to 100 bp, most preferably within 1 to 10 bp, and even most preferably immediately proximal of the first and second polynucleotides. However, the location may be even greater than 5000 bp.

Located 5′ or 3′ of components (i), (ii), and (iii): The terms “located 5′ of components (i), (ii), and (iii)” and located 3′ of the components (i), (ii), and (iii)” are defined herein as preferably within 1000 to 5000 bp, more preferably within 100 to 1000 bp, even more preferably within 10 to 100 bp, most preferably within 1 to 10 bp, and even most preferably immediately proximal of components (i), (ii), and (iii). However, the location may be even greater than 5000 bp.

Located 5′ or 3′ of the gene or a portion thereof: The terms “located 5′ of the gene or a portion thereof” and “located 3′ of the gene or a portion thereof” are defined herein as preferably within 1000 to 5000 bp, more preferably within 100 to 1000 bp, even more preferably within 10 to 100 bp, most preferably within 1 to 10 bp, and even most preferably immediately proximal of the gene or a portion thereof. However, the location may be even greater than 5000 bp.

Isolated polynucleotide: The term “isolated polynucleotide” as used herein refers to a polynucleotide that is isolated from a source. In a preferred aspect, the polynucleotide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by agarose electrophoresis.

Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. A polynucleotide of the present invention is preferably in a substantially pure form, i.e., the polynucleotide preparation is essentially free of other polynucleotide material with which it is natively or recombinantly associated. The polynucleotide may be of genomic, cDNA, RNA, semi-synthetic, synthetic origin, or any combinations thereof.

Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, recombinant nucleotide sequence, or any combination thereof.

cDNA: The term “cDNA” is defined herein as a DNA molecule that can be prepared by reverse transcription from a mature, spliced, mRNA molecule obtained from a eukaryotic cell. cDNA lacks intron sequences that are usually present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic.

Control sequences: The term “control sequences” is defined herein to include all components necessary for the expression of a polynucleotide encoding a polypeptide. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” denotes herein a configuration in which a control sequence is placed at an appropriate position relative to the coding sequence of a polynucleotide sequence such that the control sequence directs expression of the coding sequence of a polypeptide.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide and is operably linked to additional nucleotides that provide for its expression.

Introduction: The term “introduction” and variations thereof are defined herein as the transfer of a DNA into a filamentous fungal cell. The introduction of a DNA into a filamentous fungal cell can be accomplished by any method known in the art, such as transformation.

Transformation: The term “transformation” is defined herein as introducing an isolated DNA into a filamentous fungal cell so that the DNA is maintained as a chromosomal integrant or as a self-replicating extra-chromosomal vector.

Isolated polypeptide: The term “isolated polypeptide” as used herein refers to a polypeptide that is isolated from a source. In a preferred aspect, the polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially pure polypeptide: The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form, i.e., the polypeptide preparation is essentially free of other polypeptide material with which it is natively or recombinantly associated. This can be accomplished, for example, by preparing the polypeptide by well-known recombinant methods or by classical purification methods.

### DETAILED DESCRIPTION OF THE INVENTION

The present invention relates to A method for deleting a gene or a portion thereof in the genome of a filamentous fungal cell, comprising: (a) introducing into the filamentous fungal cell a nucleic acid construct comprising: (i) a first polynucleotide comprising a dominant positively selectable marker coding sequence, which when expressed confers a dominant positively selectable phenotype on the filamentous fungal cell; (ii) a second polynucleotide comprising a negatively selectable marker coding sequence, which when expressed confers a negatively selectable phenotype on the filamentous fungal cell; (iii) a first repeat sequence located 5′ of the first and second polynucleotides and a second repeat sequence located 3′ of the first and second polynucleotides, wherein the first and second repeat sequences comprise identical sequences; and (iv) a first flanking sequence located 5′ of components (i), (ii), and (iii) and a second flanking sequence located 3′ of the components (i), (ii), and (iii), wherein the first flanking sequence is identical to a first region of the genome of the filamentous fungal cell and the second flanking sequence is identical to a second region of the genome of the filamentous fungal cell, wherein (1) the first region is located 5′ of the gene or a portion thereof and the second region is located 3′ of the gene or a portion thereof of the filamentous fungal cell, (2) both of the first and second regions are located within the gene of the filamentous fungal cell, or (3) one of the first and second regions is located within the gene and the other of the first and second regions is located 5′ or 3′ of the gene of the filamentous fungal cell; wherein the first and second flanking sequences undergo intermolecular homologous recombination with the first and second regions of the filamentous fungal cell, respectively, to delete and replace the gene or a portion thereof with the nucleic acid construct; (b) selecting and isolating cells having a dominant positively selectable phenotype from step (a) by applying positive selection; and (c) selecting and isolating a cell having a negatively selectable phenotype from the selected cells having the dominant positively selectable phenotype of step (b) by applying negative selection to force the first and second repeat sequences to undergo intramolecular homologous recombination to delete the first and second polynucleotides.

In one aspect, the entire gene is completely deleted leaving no foreign DNA.

The present invention also relates to methods for introducing a polynucleotide of interest into the genome of a filamentous fungal cell, comprising: (a) introducing into the filamentous fungal cell a nucleic acid construct comprising: (i) a first polynucleotide of interest; (ii) a second polynucleotide comprising a dominant positively selectable marker coding sequence, which when expressed confers a dominant positively selectable phenotype on the filamentous fungal cell; (iii) a third polynucleotide comprising a negatively selectable marker coding sequence, which when expressed confers a negatively selectable phenotype on the filamentous fungal cell; (iv) a first repeat sequence located 5′ of the second and third polynucleotides and a second repeat sequence located 3′ of the second and third polynucleotides, wherein the first and second repeat sequences comprise identical sequences and the first polynucleotide of interest is located either 5′ of the first repeat or 3′ of the second repeat; and (v) a first flanking sequence located 5′ of components (i), (ii), (iii), and (iv) and a second flanking sequence located 3′ of the components (i), (ii), (iii), and (iv), wherein the first flanking sequence is identical to a first region of the genome of the filamentous fungal cell and the second flanking sequence is identical to a second region of the genome of the filamentous fungal cell; wherein the first and second flanking sequences undergo intermolecular homologous recombination with the first and second regions of the genome of the filamentous fungal cell, respectively, to introduce the nucleic acid construct into the genome of the filamentous fungal cell; (b) selecting cells having a dominant positively selectable phenotype from step (a) by applying positive selection; and (c) selecting and isolating a cell having a negatively selectable phenotype from the selected cells having the dominant positively selectable phenotype of step (b) by applying negative selection to force the first and second repeat sequences to undergo intramolecular homologous recombination to delete the second and third polynucleotides.

The present invention describes a bi-functional positive and negative selection system that confers on any filamentous fungus the ability to be subjected to a clean or minimally marked gene deletion or insertion. This is accomplished as a result of a transforming DNA fragment integrating into the genome and causing a gene deletion or a gene insertion from a double crossover event between the flanking DNA sequences borne on the DNA fragment and the corresponding genomic sequences of the host. Internal recombination occurs between the direct repeats resulting in the excision of the intervening sequences, with the result that a target gene in the host genome has been deleted, a polynucleotide encoding a polypeptide of interest has inserted, or a polynucleotide has inserted into a gene and either no residual DNA remains or only a single repeat remains.

In one aspect, the dual marker system provides a universal system for any filamentous fungus sensitive to hygromycin B and resistant to 5-fluoro-deoxyuridine. The present invention allows any filamentous fungal strain, which is sensitive to hygromycin B and resistant to 5-fluoro-deoxyuridine, to serve as a candidate for transformation with vectors harboring a dual positively- and negatively-selectable cassette for the purpose of (1) generating a strain harboring one or more (several) clean or minimally marked gene deletions or (2) introducing one or more (several) genes into a filamentous fungal cell, while leaving no or minimal transforming DNA in the filamentous fungal cell.

### Dominant Positively and Negatively Selectable Markers

In the methods of the present invention, any dominant positively selectable marker can be used.

In one aspect, the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble/bleO), an acetamidase gene (amdS), a pyrithiamine resistance gene (ptrA), a puromycin-N-acetyl-transferase gene (pac), an acetyl CoA synthase gene (acuA/facA), D-serine dehydratase (dsdA), an ATP sulphurylase gene (sC), a mitochondrial ATP synthase subunit 9 gene (oliC), an aminoglycoside phosphotransferase 3″(I) (aph(3′)I) gene, and an aminoglycoside phosphotransferase 3′(II) (aph(3′)II) gene.

In another aspect, the dominant positively selectable marker is encoded by a coding sequence of a hygromycin phosphotransferase gene (hpt). In another aspect, the dominant positively selectable marker is encoded by a coding sequence of a phosphinothricin acetyltransferase gene (pat). In another aspect, the dominant positively selectable marker is encoded by a coding sequence of a bleomycin, zeocin and phleomycin resistance gene (ble/bleO). In another aspect, the dominant positively selectable marker is encoded by a coding sequence of an acetamidase gene (amdS). In another aspect, the dominant positively selectable marker is encoded by a coding sequence of a pyrithiamine resistance gene (ptrA). In another aspect, the dominant positively selectable marker is encoded by a coding sequence of a puromycin-N-acetyl-transferase gene (pac). In another aspect, the dominant positively selectable marker is encoded by a coding sequence of an acetyl CoA synthase gene (acuA/facA). In another aspect, the dominant positively selectable marker is encoded by a coding sequence of a D-serine dehydratase (dsdA) gene. In another aspect, the dominant positively selectable marker is encoded by a coding sequence of an ATP sulphurylase gene (sC). In another aspect, the dominant positively selectable marker is encoded by a coding sequence of a mitochondrial ATP synthase subunit 9 gene (oliC). In another aspect, the dominant positively selectable marker is encoded by a coding sequence of an aminoglycoside phosphotransferase 3′(I) (aph(3′)I) gene. In another aspect, the dominant positively selectable marker is encoded by a coding sequence of an aminoglycoside phosphotransferase 3′(II) (aph(3′)II) gene.

The positively selectable markers can be obtained from any available source. For example, the hygromycin phosphotransferase gene (hpt) encoding hygromycin B phosphotransferase (EC 2.7.1.119; UniProtKB/Swiss-Prot P09979) can be obtained from Streptomyces hygroscopicus (Zalacain et al., 1986, Nucleic Acids Research 14: 1565-1581) and E. coli (Lino et al., 2007, Acta Crystallogr. Sect. F Struct. Biol. Cryst. Commun. 63: 685-688). The phosphinothricin acetyltransferase gene (pat or bar) encoding phosphinothricin N-acetyltransferase (EC 2.3.1.183; UniProtKB/Swiss-Prot P16426) can be obtained from Streptomyces hygroscopicus (White et al., 1990, Nucleic Acids Research 18: 1062; and Thompson et al., 1987, EMBO J. 6: 2519-2523) and Streptomyces viridochromogenes (Lutz et al., 2001, Plant Physiol. 125: 1585-1590; and Strauch et al., 1988, Gene 63: 65-74). The bleomycin resistance proteins (BRPs) encoded by, for example, ble (UniProtKB/Swiss-Prot P13081) and bleO (UniProtKB/Swiss-Prot P67925) can be obtained from Klebsiella pneumonia (Mazodier et al., 1985, Nucleic Acids Research 13: 195-205) and Bacillus stearothermophilus (Oskam et al., 1991, Plasmid 26: 30-39), respectively. The acetamidase gene (amdS) (EC 3.5.1.4; UniProtKB/Swiss-Prot P08158) can be obtained from Emericella nidulans (Aspergillus nidulans) (Corrick et al., 1987, Gene 53: 63-71), Aspergillus niger and Penicillium chrysogenum (EP 758,020). The pyrithiamine resistance gene (ptrA or thiA) encoding a mitochondrial thiazole biosynthetic enzyme (UniProtKB/Swiss-Prot Q9UUZ9) can be obtained from Aspergillus oryzae (Kubodera et al., 2000, Biosci. Biotechnol. Biochem. 64: 1416-1421). The pac gene, encoding puromycin-N-acetyl-transferase (NCBI accession no: CAB42570) can be obtained from E. coli (WO 1998/11241). The acetyl CoA synthase gene (acuA/facA; EC 6.2.1.1) can be obtained from Aspergillus niger (UniProt A2QK81), Emericella nidulans (Aspergillus nidulans) (Uniprot P16928) (Papadopoulou and Sealy-Lewis, 1999, FEMS Microbiology Letters 178: 35-37; and Sandeman and Hynes, 1989, Mol. Gen. Genet. 218: 87-92), and Phycomyces blakesleeanus (UniProtKB/Swiss-Prot Q01576) (Garre et al., 1994, Mol. Gen. Gen. 244: 278-286). The dsdA gene encoding D-serine dehydratase (EC 4.3.1.18; UniProtKB/Swiss-Prot A1ADP3) can be obtained from E. coli (Johnson et al., 2007, J. Bacteriol. 189: 3228-3236). The sC gene, encoding ATP sulfurylase (NCBI accession no: AAN04497) can be obtained from Aspergillus niger (Varadarajalu and Punekar, 2005, Microbiol. Methods. 61: 219-224). The mitochondrial ATP synthase subunit 9 (oliC) gene (UniProtKB/Swiss-Prot P16000) can be obtained from Emericella nidulans (Aspergillus nidulans) (Ward and Turner, 1986, Mol. Gen. Genet. 205: 331-338). The aminoglycoside phosphotransferase 3′(I and II) (aph(3′)I and II) genes (EC 2.7.1.95; Interpro IPR002575) can be obtained from Bacillus circulans and Streptomyces griseus (Sarwar and Akhtar, 1991, Biochem. J. 273: 807; and Trower and Clark, 1990, N.A.R. 18: 4615 respectively).

In the methods of the present invention, any negatively selectable marker can be used.

In one aspect, the negatively selectable marker is encoded by a coding sequence of a gene selected from the group consisting of a thymidine kinase gene (tk), an orotidine-5′-phosphate decarboxylase gene (pyrG), and a cytosine deaminase gene (codA).

In another aspect, the negatively selectable marker is encoded by a coding sequence of a thymidine kinase gene (tk). In another aspect, the negatively selectable marker is encoded by a coding sequence of an orotidine-5′-phosphate decarboxylase gene (pyrG). In another aspect, the negatively selectable marker is encoded by a coding sequence of a cytosine deaminase gene (codA).

The negatively selectable markers can be obtained from any available source. For example, the thymidine kinase gene (tk) (EC 2.7.1.21; UniProtKB/Swiss-Prot PO₃₁₇₆) can be obtained from human Herpes simplex virus 1 (McKnight, 1980, Nucleic Acids Research 8: 5949-5964). The orotidine-5′-phosphate decarboxylase gene (pyrG) (EC 4.1.1.23; UniProtKB/Swiss-Prot P07817) can be obtained from Aspergillus niger (Wilson et al., 1988, N.A.R. 16: 2339). The cytosine deaminase (codA) gene (EC 3.5.4.1; UniProtKB/Swiss-Prot CODA_ECOLI) can be obtained from E. coli (strain K12) (Danielsen et al., 1992, Molecular Microbiology 6: 1335-1344).

In the nucleic acid constructs, the polynucleotides encoding the positively and negatively selectable markers can be in any order relative to each other, irrespective of whether, for example, they are designated first and second polynucleotides or second and third polynucleotides. In addition, the polynucleotides encoding the positively and negatively selectable markers may be in the same orientation or in opposite orientations.

In another aspect, the dominant positively selectable marker is encoded by a coding sequence of a hygromycin phosphotransferase gene (hpt) and the negatively selectable marker is encoded by a coding sequence of a thymidine kinase gene (tk). In another aspect, the dominant positively selectable marker is encoded by a coding sequence of a phosphinothricin acetyltransferase gene (pat) and the negatively selectable marker is encoded by a coding sequence of a thymidine kinase gene (tk). In another aspect, the dominant positively selectable marker is encoded by a coding sequence of a bleomycin, zeocin and phleomycin resistance gene (ble/bleO) and the negatively selectable marker is encoded by a coding sequence of a thymidine kinase gene (tk). In another aspect, the dominant positively selectable marker is encoded by a coding sequence of an acetamidase gene (amdS) and the negatively selectable marker is encoded by a coding sequence of a thymidine kinase gene (tk). In another aspect, the dominant positively selectable marker is encoded by a coding sequence of a pyrithiamine resistance gene (ptrA) and the negatively selectable marker is encoded by a coding sequence of a thymidine kinase gene (tk). In another aspect, the dominant positively selectable marker is encoded by a coding sequence of a puromycin-N-acetyl-transferase gene (pac) and the negatively selectable marker is encoded by a coding sequence of a thymidine kinase gene (tk). In another aspect, the dominant positively selectable marker is encoded by a coding sequence of an acetyl CoA synthase gene (acuA/facA) and the negatively selectable marker is encoded by a coding sequence of a thymidine kinase gene (tk). In another aspect, the dominant positively selectable marker is encoded by a coding sequence of a D-serine dehydratase (dsdA) and the negatively selectable marker is encoded by a coding sequence of a thymidine kinase gene (tk). In another aspect, the dominant positively selectable marker is encoded by a coding sequence of an ATP sulphurylase gene (sC) and the negatively selectable marker is encoded by a coding sequence of a thymidine kinase gene (tk). In another aspect, the dominant positively selectable marker is encoded by a coding sequence of a mitochondrial ATP synthase subunit 9 gene (oliC) and the negatively selectable marker is encoded by a coding sequence of a thymidine kinase gene (tk). In another aspect, the dominant positively selectable marker is encoded by a coding sequence of an aminoglycoside phosphotransferase 3′(I) (aph(3′)I) gene and the negatively selectable marker is encoded by a coding sequence of a thymidine kinase gene (tk). In another aspect, the dominant positively selectable marker is encoded by a coding sequence of an aminoglycoside phosphotransferase 3′(II) (aph(3′)II) gene and the negatively selectable marker is encoded by a coding sequence of a thymidine kinase gene (tk).

In another aspect, the dominant positively selectable marker is encoded by a coding sequence of a hygromycin phosphotransferase gene (hpt) and the negatively selectable marker is encoded by a coding sequence of an orotidine-5′-phosphate decarboxylase gene (pyrG). In another aspect, the dominant positively selectable marker is encoded by a coding sequence of a phosphinothricin acetyltransferase gene (pat) and the negatively selectable marker is encoded by a coding sequence of an orotidine-5′-phosphate decarboxylase gene (pyrG). In another aspect, the dominant positively selectable marker is encoded by a coding sequence of a bleomycin, zeocin and phleomycin resistance gene (ble/bleO) and the negatively selectable marker is encoded by a coding sequence of an orotidine-5′-phosphate decarboxylase gene (pyrG). In another aspect, the dominant positively selectable marker is encoded by a coding sequence of an acetamidase gene (amdS) and the negatively selectable marker is encoded by a coding sequence of an orotidine-5′-phosphate decarboxylase gene (pyrG). In another aspect, the dominant positively selectable marker is encoded by a coding sequence of a pyrithiamine resistance gene (ptrA) and the negatively selectable marker is encoded by a coding sequence of an orotidine-5′-phosphate decarboxylase gene (pyrG). In another aspect, the dominant positively selectable marker is encoded by a coding sequence of a puromycin-N-acetyl-transferase gene (pac) and the negatively selectable marker is encoded by a coding sequence of an orotidine-5′-phosphate decarboxylase gene (pyrG). In another aspect, the dominant positively selectable marker is encoded by a coding sequence of an acetyl CoA synthase gene (acuA/facA) and the negatively selectable marker is encoded by a coding sequence of an orotidine-5′-phosphate decarboxylase gene (pyrG). In another aspect, the dominant positively selectable marker is encoded by a coding sequence of a D-serine dehydratase (dsdA) and the negatively selectable marker is encoded by a coding sequence of an orotidine-5′-phosphate decarboxylase gene (pyrG). In another aspect, the dominant positively selectable marker is encoded by a coding sequence of an ATP sulphurylase gene (sC) and the negatively selectable marker is encoded by a coding sequence of an orotidine-5′-phosphate decarboxylase gene (pyrG). In another aspect, the dominant positively selectable marker is encoded by a coding sequence of a mitochondrial ATP synthase subunit 9 gene (oliC) and the negatively selectable marker is encoded by a coding sequence of an orotidine-5′-phosphate decarboxylase gene (pyrG). In another aspect, the dominant positively selectable marker is encoded by a coding sequence of an aminoglycoside phosphotransferase 3′(I) (aph(3′)I) gene and the negatively selectable marker is encoded by a coding sequence of an orotidine-5′-phosphate decarboxylase gene (pyrG). In another aspect, the dominant positively selectable marker is encoded by a coding sequence of an aminoglycoside phosphotransferase 3′(II) (aph(3′)II) gene and the negatively selectable marker is encoded by a coding sequence of an orotidine-5′-phosphate decarboxylase gene (pyrG).

In another aspect, the dominant positively selectable marker is encoded by a coding sequence of a hygromycin phosphotransferase gene (hpt) and the negatively selectable marker is encoded by a coding sequence of a cytosine deaminase gene (codA). In another aspect, the dominant positively selectable marker is encoded by a coding sequence of a phosphinothricin acetyltransferase gene (pat) and the negatively selectable marker is encoded by a coding sequence of a cytosine deaminase gene (codA). In another aspect, the dominant positively selectable marker is encoded by a coding sequence of a bleomycin, zeocin and phleomycin resistance gene (ble/bleO) and the negatively selectable marker is encoded by a coding sequence of a cytosine deaminase gene (codA). In another aspect, the dominant positively selectable marker is encoded by a coding sequence of an acetamidase gene (amdS) and the negatively selectable marker is encoded by a coding sequence of a cytosine deaminase gene (codA). In another aspect, the dominant positively selectable marker is encoded by a coding sequence of a pyrithiamine resistance gene (ptrA) and the negatively selectable marker is encoded by a coding sequence of a cytosine deaminase gene (codA). In another aspect, the dominant positively selectable marker is encoded by a coding sequence of a puromycin-N-acetyl-transferase gene (pac) and the negatively selectable marker is encoded by a coding sequence of a cytosine deaminase gene (codA). In another aspect, the dominant positively selectable marker is encoded by a coding sequence of an acetyl CoA synthase gene (acuA/facA) and the negatively selectable marker is encoded by a coding sequence of a cytosine deaminase gene (codA). In another aspect, the dominant positively selectable marker is encoded by a coding sequence of a D-serine dehydratase (dsdA) and the negatively selectable marker is encoded by a coding sequence of a cytosine deaminase gene (codA). In another aspect, the dominant positively selectable marker is encoded by a coding sequence of an ATP sulphurylase gene (sC) and the negatively selectable marker is encoded by a coding sequence of a cytosine deaminase gene (codA). In another aspect, the dominant positively selectable marker is encoded by a coding sequence of a mitochondrial ATP synthase subunit 9 gene (oliC) and the negatively selectable marker is encoded by a coding sequence of a cytosine deaminase gene (codA). In another aspect, the dominant positively selectable marker is encoded by a coding sequence of an aminoglycoside phosphotransferase 3″(I) (aph(3′)II) gene and the negatively selectable marker is encoded by a coding sequence of a cytosine deaminase gene (codA). In another aspect, the dominant positively selectable marker is encoded by a coding sequence of an aminoglycoside phosphotransferase 3′(II) (aph(3′)II) gene and the negatively selectable marker is encoded by a coding sequence of a cytosine deaminase gene (codA).

The present invention also relates to an isolated orotidine-5′-phosphate decarboxylase selected from the group consisting of: (a) an orotidine-5′-phosphate decarboxylase comprising an amino acid sequence having preferably at least 70%, more preferably at least 75%, more preferably at least 80%, more preferably at least 85%, even more preferably at least 90%, even more preferably at least 95% identity, and most preferably at least 95%, at least 97%, at least 98%, or at least 99% identity to the mature polypeptide of SEQ ID NO: 52; (b) an orotidine-5′-phosphate decarboxylase encoded by a polynucleotide that hybridizes under preferably at least medium stringency conditions, more preferably at least medium stringency conditions, even more preferably at least high stringency conditions, and most preferably very high stringency conditions with the mature polypeptide coding sequence of SEQ ID NO: 51 or its full-length complementary strand; and (c) an orotidine-5′-phosphate decarboxylase encoded by a polynucleotide comprising a nucleotide sequence having preferably at least 80%, more preferably at least 85%, even more preferably at least 90%, even more preferably at least 95% identity, and most preferably, at least 96%, at least 97%, at least 98%, or at least 99% identity to the mature polypeptide coding sequence of SEQ ID NO: 51.

In a preferred aspect, the orotidine-5′-phosphate decarboxylase comprises or consists of SEQ ID NO: 52 or a fragment thereof having orotidine-5′-phosphate decarboxylase activity. In another preferred aspect, the orotidine-5′-phosphate decarboxylase comprises or consists of SEQ ID NO: 52.

The present invention also relates to an isolated polynucleotide comprising a nucleotide sequence encoding an orotidine-5′-phosphate decarboxylase selected from the group consisting of: (a) a polynucleotide comprising a nucleotide sequence encoding an orotidine-5′-phosphate decarboxylase comprising an amino acid sequence having preferably at least 70%, more preferably at least 75%, more preferably at least 80%, more preferably at least 85%, even more preferably at least 90%, even more preferably at least 95% identity, and most preferably at least 95%, at least 97%, at least 98%, or at least 99% identity to the mature polypeptide of SEQ ID NO: 52; (b) a polynucleotide encoding an orotidine-5′-phosphate decarboxylase comprising a nucleotide sequence that hybridizes under preferably at least medium stringency conditions, more preferably at least medium stringency conditions, even more preferably at least high stringency conditions, and most preferably very high stringency conditions with SEQ ID NO: 51 or its full-length complementary strand; and (c) a polynucleotide encoding an orotidine-5′-phosphate decarboxylase comprising a nucleotide sequence having preferably at least 80%, more preferably at least 85%, even more preferably at least 90%, even more preferably at least 95% identity, and most preferably, at least 96%, at least 97%, at least 98%, or at least 99% identity to the mature polypeptide coding sequence of SEQ ID NO: 51.

In a preferred aspect, a polynucleotide encoding an orotidine-5′-phosphate decarboxylase comprises or consists of SEQ ID NO: 51 or a subsequence thereof that encodes a fragment having orotidine-5′-phosphate decarboxylase activity. In another preferred aspect, a polynucleotide encoding an orotidine-5′-phosphate decarboxylase comprises or consists of SEQ ID NO: 51.

Techniques used to isolate or clone a polynucleotide encoding a polypeptide are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used.

The nucleotide sequence of SEQ ID NO: 51; or a subsequence thereof; as well as the amino acid sequence of SEQ ID NO: 52; or a fragment thereof; may be used to design nucleic acid probes to identify and clone DNA encoding orotidine-5′-phosphate decarboxylases from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes that are preferably at least 600 nucleotides, more preferably at least 700 nucleotides, even more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from a strain may, therefore, be screened for DNA that hybridizes with the probes described above and encodes an orotidine-5′-phosphate decarboxylase. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1, or a subsequence thereof, the carrier material is preferably used in a Southern blot.

For purposes of the present invention, hybridization indicates that the nucleotide sequence hybridizes to a labeled nucleic acid probe corresponding to SEQ ID NO: 51 or a subsequence thereof; under very low to very high stringency conditions. Molecules to which the nucleic acid probe hybridizes under these conditions can be detected using, for example, X-ray film.

In a preferred aspect, the nucleic acid probe is SEQ ID NO: 51. In another preferred aspect, the nucleic acid probe is a polynucleotide sequence that encodes SEQ ID NO: 52, or a subsequence thereof. In another preferred aspect, the nucleic acid probe is a polynucleotide sequence that encodes SEQ ID NO: 52.

For probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For probes of at least 100 nucleotides in length, the carrier material is finally washed three times each for 15 minutes using 2×SSC, 0.2% SDS preferably at 45° C. (very low stringency), more preferably at 50° C. (low stringency), more preferably at 55° C. (medium stringency), more preferably at 60° C. (medium-high stringency), even more preferably at 65° C. (high stringency), and most preferably at 70° C. (very high stringency).

The present invention also relates to nucleic acid constructs, recombinant expression vectors, and recombinant filamentous fungal cells comprising such an orotidine-5′-phosphate decarboxylase.

The present invention also relates to methods of producing the orotidine-5′-phosphate decarboxylase, comprising: cultivating a host cell comprising a nucleic acid construct comprising a nucleotide sequence encoding the orotidine-5′-phosphate decarboxylase under conditions conducive for production of the polypeptide. In a preferred aspect, the host cell is a filamentous fungal cell.

### Repeat Sequences

In the methods of the present invention for deleting a gene in the genome of a filamentous fungus, the nucleic acid construct comprising a first polynucleotide encoding a dominant positively selectable marker and a second polynucleotide encoding a negatively selectable marker also comprises a first repeat sequence located 5′ of the first and second polynucleotides and a second repeat sequence located 3′ of the first and second polynucleotides.

In the methods of the present invention for introducing a polynucleotide of interest into the genome of a filamentous fungus, the nucleic acid construct comprising a first polynucleotide of interest, a second polynucleotide encoding a dominant positively selectable marker, and a third polynucleotide encoding a negatively selectable marker also comprises a first repeat sequence located 5′ of the second and third polynucleotides and a second repeat sequence located 3′ of the second and third polynucleotides, wherein the first polynucleotide of interest is located either 5′ of the first repeat or 3′ of the second repeat.

The repeat sequences for both methods preferably comprise identical sequences so the first and second repeat sequences can undergo intramolecular homologous recombination to delete the polynucleotides encoding the positively and negatively selectable markers.

The repeat sequences can be any polynucleotide sequence. In one aspect, the repeat sequences are sequences native to the filamentous fungal cell. In another aspect, the repeat sequences are sequences foreign (heterologous) to the filamentous fungal cell. The repeat sequences may be non-encoding or encoding polynucleotide sequences. In another aspect, the repeat sequences are polynucleotide sequences native to the filamentous fungal cell. In another aspect, the repeat sequences are identical to either the 3′ flanking sequence or the 5′ flanking to insure a clean gene deletion, disruption, or insertion.

To increase the likelihood of intramolecular homologous recombination to delete the polynucleotides for the positively and negatively selectable markers, the repeat sequences should contain a sufficient number of nucleic acids, such as preferably 20 to 10,000 base pairs, 50 to 10,000 base pairs, 100 to 10,000 base pairs, 200 to 10,000 base pairs, more preferably 400 to 10,000 base pairs, and most preferably 800 to 10,000 base pairs.

### Flanking Sequences

In the methods of the present invention for deleting a gene of interest in the genome of a filamentous fungus, the nucleic acid construct comprising a first polynucleotide encoding a dominant positively selectable marker, a second polynucleotide encoding a negatively selectable marker, a first repeat sequence, and a second repeat sequence also comprises a first flanking sequence located 5′ of the above-noted polynucleotides and a second flanking sequence located 3′ of the above-noted polynucleotides.

For deleting a gene of interest, the first flanking sequence is identical to a first region located at the 5′ end of the gene of the filamentous fungal cell and the second flanking sequence is identical to a second region located at the 3′ end of the gene. The first and second flanking sequences undergo intermolecular homologous recombination with the first and second regions of the genome of the filamentous fungal cell, respectively, to delete and replace the gene with the nucleic acid construct.

In the methods of the present invention for introducing a polynucleotide of interest into the genome of a filamentous fungus, the nucleic acid construct comprising the polynucleotide of interest, a second polynucleotide encoding a dominant positively selectable marker, a third polynucleotide encoding a negatively selectable marker, a first repeat sequence, and a second repeat sequence also comprises a first flanking sequence located 5′ of the above-noted polynucleotides and a second flanking sequence located 3′ of the above-noted polynucleotides.

For introducing a polynucleotide of interest, the first flanking sequence is identical to a first region of the genome of the filamentous fungal cell and the second flanking sequence is identical to a second region of the genome of the filamentous fungal cell. The first and second flanking sequences undergo intermolecular homologous recombination with the first and second regions of the genome of the filamentous fungal cell, respectively, to introduce the nucleic acid construct comprising the polynucleotide of interest into the genome of the filamentous fungal cell.

In one aspect, the first region is located 5′ of the gene and the second region is located 3′ of the gene of the filamentous fungal cell. In another aspect, both of the first and second regions are located within a gene of the filamentous fungal cell. In another aspect, one of the first and second regions is located within a gene and the other of the first and second regions is located 5′ or 3′ of the gene of the filamentous fungal cell.

In another aspect, the first and second repeat sequences are identical to either the first flanking sequence or the second flanking sequence.

To increase the likelihood of integration at a precise location, the flanking sequences should preferably contain a sufficient number of nucleic acids, such as 100 to 10,000 base pairs, preferably 400 to 10,000 base pairs, and most preferably 800 to 10,000 base pairs, sufficient to insure homologous recombination. The flanking sequences may be any sequence that is identical with the target sequence in the genome of the filamentous fungal cell. Furthermore, the flanking sequences may be non-encoding or encoding nucleotide sequences.

### Polynucleotides

In the methods of the present invention, the polynucleotide of interest can be any DNA. The DNA may be native or heterologous (foreign) to the filamentous fungal cell of interest.

The polynucleotide may encode any polypeptide having a biological activity of interest. The polypeptide may be native or heterologous (foreign) to the filamentous fungal cell of interest. The term “heterologous polypeptide” is defined herein as a polypeptide that is not native to the filamentous fungal cell; a native polypeptide in which structural modifications, e.g., deletions, substitutions, and/or insertions, have been made to alter the native polypeptide; or a native polypeptide whose expression is quantitatively altered as a result of manipulation of the DNA encoding the polypeptide by recombinant DNA techniques, e.g., a stronger promoter. The polypeptide may be a naturally occurring allelic and engineered variations of the below-mentioned polypeptides and hybrid polypeptides.

The term “polypeptide” is not meant herein to refer to a specific length of the encoded product and, therefore, encompasses peptides, oligopeptides, and proteins. The term “polypeptide” also encompasses hybrid polypeptides and fusion polypeptides. Polypeptides further include naturally occurring allelic and engineered variations of a polypeptide.

In one aspect, the polypeptide is an antibody, antigen, antimicrobial peptide, enzyme, growth factor, hormone, immunodilator, neurotransmitter, receptor, reporter protein, structural protein, and transcription factor.

In another aspect, the polypeptide is an oxidoreductase, transferase, hydrolase, lyase, isomerase, or ligase. In another aspect, the polypeptide is an alpha-glucosidase, aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, glucocerebrosidase, alpha-glucosidase, beta-glucosidase, invertase, laccase, lipase, mannosidase, mutanase, oxidase, pectinolytic enzyme, peroxidase, phospholipase, phytase, polyphenoloxidase, proteolytic enzyme, ribonuclease, transglutaminase, urokinase, or xylanase.

In another aspect, the polypeptide is an albumin, collagen, tropoelastin, elastin, or gelatin.

In another aspect, the polypeptide is a hybrid polypeptide, which comprises a combination of partial or complete polypeptide sequences obtained from at least two different polypeptides wherein one or more may be heterologous to the filamentous fungal cell.

In another aspect, the polypeptide is a fused polypeptide in which another polypeptide is fused at the N-terminus or the C-terminus of the polypeptide or fragment thereof. A fused polypeptide is produced by fusing a nucleotide sequence (or a portion thereof) encoding one polypeptide to a nucleotide sequence (or a portion thereof) encoding another polypeptide. Techniques for producing fusion polypeptides are known in the art, and include, ligating the coding sequences encoding the polypeptides so that they are in frame and expression of the fused polypeptide is under control of the same promoter(s) and terminator.

The polynucleotide encoding a polypeptide of interest may be obtained from any prokaryotic, eukaryotic, or other source. For purposes of the present invention, the term “obtained from” as used herein in connection with a given source shall mean that the polypeptide is produced by the source or by a cell in which a gene from the source has been inserted.

The techniques used to isolate or clone a polynucleotide encoding a polypeptide of interest are known in the art and include isolation from genomic DNA, preparation from cDNA, or a combination thereof. The cloning of the polynucleotide of interest from such genomic DNA can be effected, e.g., by using the well known polymerase chain reaction (PCR). See, for example, Innis et al., 1990, PCR Protocols: A Guide to Methods and Application, Academic Press, New York. The cloning procedures may involve excision and isolation of a desired nucleic acid fragment comprising the nucleic acid sequence encoding the polypeptide, insertion of the fragment into a vector molecule, and incorporation of the recombinant vector into the mutant fungal cell where multiple copies or clones of the nucleic acid sequence will be replicated. The polynucleotide may be of genomic, cDNA, RNA, semi-synthetic, synthetic origin, or any combinations thereof.

A polynucleotide encoding a polypeptide of interest may be manipulated in a variety of ways to provide for expression of the polynucleotide in a suitable filamentous fungal cell. The construction of nucleic acid constructs and recombinant expression vectors for the DNA encoding a polypeptide of interest can be carried out as described herein.

The polynucleotide may also be a control sequence, e.g., promoter, for manipulating the expression of a gene of interest. Non-limiting examples of control sequences are described herein.

The polynucleotide may also be any nucleic acid molecule useful for disrupting a gene in the genome of the filamentous fungus. The polynucleotide may be a coding or non-coding polynucleotide. The polynucleotide may encode another selectable marker besides those disclosed earlier. The polynucleotide may encode a polypeptide such as those described above. The polynucleotide may simply be any nucleic acid molecule of sufficient length to disrupt the gene.

The polynucleotide is not to be limited in scope by the specific examples disclosed above, since these examples are intended as illustrations of several aspects of the invention.

### Nucleic Acid Constructs

The present invention also relates to nucleic acid constructs for deleting a gene or a portion thereof in the genome of a filamentous fungal cell, comprising: (i) a first polynucleotide comprising a dominant positively selectable marker coding sequence, which when expressed confers a dominant positively selectable phenotype on the filamentous fungal cell; (ii) a second polynucleotide comprising a negatively selectable marker coding sequence, which when expressed confers a negatively selectable phenotype on the filamentous fungal cell; (iii) a first repeat sequence located 5′ of the first and second polynucleotides and a second repeat sequence located 3′ of the first and second polynucleotides, wherein the first and second repeat sequences comprise identical sequences; and (iv) a first flanking sequence located 5′ of components (i), (ii), and (iii) and a second flanking sequence located 3′ of the components (i), (ii), and (iii), wherein the first flanking sequence is identical to a first region of the genome of the filamentous fungal cell and the second flanking sequence is identical to a second region of the genome of the filamentous fungal cell, wherein (1) the first region is located 5′ of the gene or a portion thereof and the second region is located 3′ of the gene or a portion thereof of the filamentous fungal cell, (2) both of the first and second regions are located within the gene of the filamentous fungal cell, or (3) one of the first and second regions is located within the gene and the other of the first and second regions is located 5′ or 3′ of the gene of the filamentous fungal cell, wherein the first and second flanking sequences undergo intermolecular homologous recombination with the first and second regions of the filamentous fungal cell, respectively, to delete and replace the gene or a portion thereof with the nucleic acid construct

The present invention also relates to nucleic acid constructs for introducing a polynucleotide into the genome of a filamentous fungal cell, comprising (i) a first polynucleotide of interest; (ii) a second polynucleotide comprising a dominant positively selectable marker coding sequence, which when expressed confers a dominant positively selectable phenotype on the filamentous fungal cell; (iii) a third polynucleotide comprising a negatively selectable marker coding sequence, which when expressed confers a negatively selectable phenotype on the filamentous fungal cell; (iv) a first repeat sequence located 5′ of the second and third polynucleotides and a second repeat sequence located 3′ of the second and third polynucleotides, wherein the first and second repeat sequences comprise identical sequences and the first polynucleotide of interest is located either 5′ of the first repeat or 3′ of the second repeat; and (v) a first flanking sequence located 5′ of components (i), (ii), (iii), and (iv) and a second flanking sequence located 3′ of the components (i), (ii), (iii), and (iv), wherein the first flanking sequence is identical to a first region of the genome of the filamentous fungal cell and the second flanking sequence is identical to a second region of the genome of the filamentous fungal cell; the first and second flanking sequences undergo intermolecular homologous recombination with the first and second regions of the genome of the filamentous fungal cell, respectively, to introduce the nucleic acid construct into the genome of the filamentous fungal cell; and the first and second repeat sequences can undergo intramolecular homologous recombination to delete the second and third polynucleotides.

An isolated polynucleotide encoding a polypeptide of interest, a dominant positively selectable marker, or a negatively selectable marker may be manipulated in a variety of ways to provide for its expression. Manipulation of such a polynucleotide's sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence that is recognized by a filamentous fungal cell for expression of a polynucleotide encoding a polypeptide of interest. The promoter sequence contains transcriptional control sequences that mediate the expression of the polypeptide. The promoter may be any nucleotide sequence that shows transcriptional activity in the filamentous fungal cell of choice including mutant, truncated, and hybrid promoters, and may be obtained from genes encoding extracellular or intracellular polypeptides either homologous or heterologous to the filamentous fungal cell.

Examples of suitable promoters for directing the transcription of the nucleic acid constructs of the present invention in a filamentous fungal cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum amyA, Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

The control sequence may also be a suitable transcription terminator sequence, a sequence recognized by a filamentous fungal cell to terminate transcription. The terminator sequence is operably linked to the 3′ terminus of a nucleotide sequence encoding a polypeptide. Any terminator that is functional in the filamentous fungal cell of choice may be used in the present invention.

Preferred terminators for filamentous fungal cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

The control sequence may also be a suitable leader sequence, a nontranslated region of an mRNA that is important for translation by the filamentous fungal cell. The leader sequence is operably linked to the 5′ terminus of a nucleotide sequence encoding a polypeptide. Any leader sequence that is functional in the filamentous fungal cell of choice may be used in the present invention.

Preferred leaders for filamentous fungal cells are obtained from the genes for Aspergillus oryzae TAKA amylase and Aspergillus nidulans triose phosphate isomerase.

The control sequence may also be a polyadenylation sequence, a sequence operably linked to the 3′ terminus of a nucleotide sequence and, when transcribed, is recognized by the filamentous fungal cell as a signal to add polyadenosine residues to transcribed mRNA. Any polyadenylation sequence that is functional in the filamentous fungal cell of choice may be used in the present invention.

Preferred polyadenylation sequences for filamentous fungal cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide coding sequence that encodes a signal peptide linked to the amino terminus of a polypeptide and directs the encoded polypeptide into the cell's secretory pathway. The 5′ end of the coding sequence of the nucleotide sequence may inherently contain a signal peptide coding sequence naturally linked in translation reading frame with the segment of the coding sequence that encodes the secreted polypeptide. Alternatively, the 5′ end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of a filamentous fungal cell of choice, i.e., secreted into a culture medium, may be used in the present invention.

Effective signal peptide coding sequences for filamentous fungal cells are the signal peptide coding sequences obtained from the genes for Aspergillus oryzae TAKA amylase, Aspergillus niger neutral amylase, Aspergillus niger glucoamylase, Rhizomucor miehei aspartic proteinase, Humicola insolens cellulase, Humicola insolens endoglucanase V, and Humicola lanuginosa lipase.

The control sequence may also be a propeptide coding sequence that encodes a propeptide positioned at the amino terminus of a polypeptide. The resultant polypeptide is known as a proenzyme or propolypeptide (or a zymogen in some cases). A propeptide is generally inactive and can be converted to a mature active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide sequences are present at the amino terminus of a polypeptide, the propeptide sequence is positioned next to the amino terminus of a polypeptide and the signal peptide sequence is positioned next to the amino terminus of the propeptide sequence.

It may also be desirable to add regulatory sequences that allow the regulation of the expression of a polypeptide relative to the growth of the filamentous fungal cell. Examples of regulatory systems are those that cause the expression of the gene to be turned on or off in response to a chemical or physical stimulus, including the presence of a regulatory compound. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the nucleotide sequence encoding the polypeptide would be operably linked with the regulatory sequence.

### Expression Vectors

The present invention also relates to recombinant expression vectors comprising a nucleic acid construct of the present invention. The recombinant expression vector may be any plasmid that can be conveniently subjected to recombinant DNA procedures and can bring about expression of the polynucleotide sequences. The choice of the vector will typically depend on the compatibility of the vector with the filamentous fungal cell into which the vector is to be introduced. The vectors are preferably linear so that the first and second flanking sequences undergo efficient intermolecular homologous recombination with the first and second regions of the filamentous fungal cell.

The procedures used to construct the recombinant expression vectors of the present invention are well known to one skilled in the art (see, e.g., Sambrook et al., 1989, supra).

### Filamentous Fungal Cells

The present invention also relates to recombinant filamentous fungal cells comprising ae nucleic acid construct of the invention.

In the methods of the present invention, the filamentous fungal cell may be any filamentous fungal cell. The term “filamentous fungal cell” encompasses any progeny of a parent cell that is not identical to the parent cell due to mutations that occur during replication.

“Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In one aspect, the filamentous fungal cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a more preferred aspect, the filamentous fungal cell is an Aspergillus awamori, Aspergillus fumigatus, Aspergillus foetidus, Aspergillus japonicus, Aspergillus nidulans, Aspergillus niger or Aspergillus oryzae cell. In another more preferred aspect, the filamentous fungal cell is a Fusarium bactridioides, Fusarium cerealis, Fusarium crookwellense, Fusarium culmorum, Fusarium graminearum, Fusarium graminurn, Fusarium heterosporum, Fusarium negundi, Fusarium oxysporum, Fusarium reticulaturn, Fusarium roseum, Fusarium sambucinurn, Fusarium sarcochroum, Fusarium sporotrichioides, Fusarium sulphureum, Fusarium torulosum, Fusarium trichothecioides, or Fusarium venenatum cell. In another more preferred aspect, the filamentous fungal cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

In a most preferred aspect, the filamentous fungal cell is a Fusarium venenatum cell. In another most preferred aspect, the filamentous fungal cell is Fusarium venenatum NRRL 30747. In another most preferred aspect, the filamentous fungal cell is Fusarium venenatum ATCC 20334.

In another most preferred aspect, the filamentous fungal cell is an Aspergillus niger cell.

In another most preferred aspect, the filamentous fungal cell is an Aspergillus oryzae cell.

In another most preferred aspect, the filamentous fungal cell is a Trichoderma reesei cell.

Filamentous fungal cells may be transformed by a process involving protoplast formation, transformation of the protoplasts, and regeneration of the cell wall in a manner known per se. Suitable procedures for transformation of Aspergillus and Trichoderma cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787.

### Methods of Production

The present invention also relates to methods of producing a polypeptide of interest, comprising: (a) cultivating a filamentous fungal cell, obtained as described herein, under conditions conducive for production of the polypeptide; and (b) recovering the polypeptide.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted into the medium, it can be recovered from cell lysates.

The polypeptide may be detected using methods known in the art that are specific for the polypeptide. These detection methods may include use of specific antibodies, formation of an enzyme product, or disappearance of an enzyme substrate. For example, an enzyme assay may be used to determine the activity of the polypeptide.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptide of interest may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J. -C. Janson and Lars Ryden, editors, VCH Publishers, New York, 1989) to obtain a substantially pure polypeptide.

The present invention is further described by the following examples that should not be construed as limiting the scope of the invention.

### EXAMPLES

Materials

Chemicals used as buffers and substrates were commercial products of at least reagent grade. All primers and oligonucleotides were supplied by MWG Biotech, Inc., High Point, N.C., USA.

### Fungal Strains

Fusarium venenatum strain WTY842-1-11 is described in U.S. Pat. No. 7,368,271. Fusarium venenatum strain EmY1154-46-4.3 is a Δtri5, amdS+, ΔpyrG derivative of Fusarium venenatum strain WTY842-1-11. Fusarium venenatum strain WTY1449-03-03 is a Δtri5, amdS+, bar+, tk+transformant of Fusarium venenatum strain WTY842-1-11. Fusarium venenatum strain WTY1449-09-01 is a Δtri5, amdS+, bar+, tk-cured derivative of Fusarium venenatum strain WTY1449-03-03. Fusarium strain A3/5, now reclassified as Fusarium venenatum (Yoder and Christianson, 1998, Fungal Genetics and Biology 23: 62-80; O'Donnell et al., 1998, Fungal Genetics and Biology 23: 57-67), was obtained from Dr. Anthony Trinci, University of Manchester, Manchester, England. Deposits of this strain can be obtained from the American Type Culture Collection, Manassas, Va., USA as Fusarium strain ATCC 20334 or the Agricultural Research Service Patent Culture Collection (NRRL), Northern Regional Research Center, Peoria, Ill., USA as Fusarium strain NRRL 30747. Trichoderma reesei RutC30 is described by Montenecourt and Eveleigh, 1979, Adv. Chem. Ser. 181: 289-301.

### Media and Solutions

LB plates were composed per liter of 10 g of tryptone, 5 g of yeast extract, 5 g of NaCl, and 15 g of Bacto agar.

NZY top agarose was composed per liter of 5 g of NaCl, 5 g of yeast extract, 10 g of NZ amine, 2 g of MgSO₄, and 7 g of agarose.

M400 medium was composed per liter of 50 g of maltodextrin, 2 g of MgSO₄.7H₂O, 2 g of KH₂PO₄, 4 g of citric acid, 8 g of yeast extract, 2 g of urea, 0.5 g of CaCl₂, and 0.5 ml of AMG trace metals solution, pH 6.0.

AMG trace metals solution were composed per liter of 14.3 g of ZnSO₄.7H₂O, 2.5 g of CuSO₄.5H₂O, 0.5 g of NiCl₂, 13.8 g of FeSO₄, 8.5 g of MnSO₄, and 3.0 g of citric acid.

2XYT medium was composed per liter of 16 g of tryptone, 10 g of yeast extract, 5 g of NaCl, and 5 g of Bacto agar.

YP medium was composed per liter of 10 g of yeast extract and 20 g of Bacto peptone.

YPG_{2%} medium was composed per liter of 10 g of yeast extract, 20 g of Bacto peptone, and 20 g of glucose.

YPG_{5%} medium was composed per liter of 10 g of yeast extract, 20 g of Bacto peptone, and 50 g of glucose.

RA medium was composed per liter of 50 g of succinic acid, 12.1 g of NaNO₃, 1 g of glucose, and 20 ml of 50× Vogels salts solution (No C, No NaNO₃).

RA+uridine medium was composed per liter of 50 g of succinic acid, 12.1 g of NaNO₃, 1 g of glucose, and 20 ml of 50× Vogels salts solution (No C, No NaNO₃). After filter sterilization of the RA medium, filter sterilized uridine was added to a final concentration of 10 mM.

RA+BASTA™ medium was composed per liter of 50 g of succinic acid, 12.1 g of NaNO₃, 1 g of glucose, and 20 ml of 50× Vogels salts solution (No C, No NaNO₃). After filter sterilization of the RA medium, filter-sterilized BASTA™ (glufosinate, Hoechst Schering AgrEvo, Frankfurt, Germany) was added to a final concentration of 6 mg/ml using a working stock solution of 250 mg/ml.

50× Vogels salts solution (No C, No NaNO₃) was composed of per liter of 250 g of KH₂PO₄, 10 g of MgSO₄.7H₂O, 5 g of CaCl₂2H₂O, 2.5 ml of biotin solution, and 5 ml of Vogels trace elements solution.

Biotin stock solution was composed of 5 mg of biotin in 100 ml of 50% ethanol.

Vogels trace elements solution was composed per 100 ml of 5 g of citric acid, 5 g of ZnSO₄.7H₂O, 1 g of Fe(NH₄)₂(SO₄)₂.6H₂O, 0.25 g of CuSO₄.5H₂O, 0.05 g of MnSO₄.H₂O, 0.05 g of H₃BO₃, and 0.05 g of Na₂MoO₄.2H₂O.

PDA plates were composed per liter of 39 g of Potato Dextrose Agar (BD Biosciences, San Jose, Calif., USA)

PDA+1 M sucrose plates were composed per liter of 39 g of Potato Dextrose Agar (BD Biosciences, San Jose, Calif., USA) and 342 g of sucrose.

VNO₃RLMT plates were composed per liter of 20 ml of 50× Vogels salts solution (25 mM NaNO₃), 273.33 g of sucrose, and 15 g of LMT agarose (Sigma, St. Louis, Mo., USA).

50× Vogels salts solution (25 mM NaNO₃) was composed per liter of 125 g of sodium citrate, 250 g of KH₂PO₄, 106.25 g of NaNO₃, 10 g of MgSO₄.7H₂O, 5 g of CaCl₂2H₂O, 2.5 ml of biotin stock solution, and 5 ml of Vogels trace elements solution.

VNO₃RLMT-BASTA™ plates were composed per liter of 20 ml of 50× Vogels salts solution (25 mM NaNO₃), 273.33 g of sucrose, and 15 g of LMT agarose. After autoclaving and cooling BASTA™ was added to a final concentration of 6 mg/ml.

COVE salt solution was composed of 26 g KCl, 26 g MgSO₄ 7H₂0, 76 g KH₂PO₄, 50 ml COVE trace elements, per liter.

COVE trace elements solution was composed of 0.004 g of Na₂B₄O₇10H₂O, 0.4 g of CuSO₄5H₂O, 1.2 g of FeSO₄7H₂O, 0.7 g of MnSO₄H₂O, 0.8 g Na₂MoO₂2H₂O, 10 g of ZnSO₄7H₂O, per liter.

TrMM medium was composed of 20 ml of COVE salt solution, 0.6 g of CaCl₂, 6 g of (NH₄)₂SO₄, 30 g of sucrose, and 25 g Agar Noble

TrMM-G was composed of 20 ml of COVE salt solution, 0.6 g of CaCl₂, 6 g of (NH₄)₂SO₄, 25 g of Agar Noble, autoclaved, cooled and 40 ml of 50% glucose added.

STC was composed of 0.8 M sorbitol, 2.5 mM Tris pH 8, and 5 mM CaCl₂.

TrSTC was composed of 1 M sorbitol, 10 mM Tris pH 8, and 10 mM CaCl₂.

PEG was composed of 50% PEG 4000, 10 mM Tris pH7.5 and 10 mM CaCl₂

STC was composed of 0.8 M sorbitol, 25 or 50 mM Tris pH 8, and 50 mM CaCl₂.

SPTC was composed of 40% polyethylene glycol 4000, 0.8 M sorbitol, 25 or 50 mM Tris pH 8, and 50 mM CaCl₂.

SY50 medium (pH 6.0) was composed per liter of 50 g of sucrose, 2.0 g of MgSO₄.7H₂O, 10 g of KH₂PO₄, 2.0 g of K₂SO₄, 2.0 g of citric acid, 10 g of yeast extract, 2.0 g of urea, 0.5 g of CaCl₂.2H₂O, and 5 ml of 200×AMG trace metals solution (no nickel).

200×AMG trace metals solution (no nickel) was composed per liter of 3.0 g of citric acid, 14.3 g of ZnSO₄.7H₂O, 2.5 g of CuSO₄.5H₂O, 13.8 g of FeSO₄.7H₂O, and 8.5 g of MnSO₄.H₂O.

20×SSC was composed of 0.3 M sodium citrate pH 7 and 3 M sodium chloride.

### DNA Sequencing

DNA sequencing was conducted with an ABI PRIZM® 3700 DNA Analyzer (Applied Biosystems, Inc., Foster City, Calif., USA).

### Example 1

### Sensitivity Testing of Fusarium venenatum WTY842-1-11 to 5-fluoro-deoxyuridine (FdU)

For a thymidine kinase gene (tk) to be useful as a negatively selectable marker, a fungus must be insensitive to rather high concentrations of the nucleoside analog 5-fluoro-deoxyuridine (FdU). In order to ascertain the degree of sensitivity of Fusarium venenatum WTY842-1-11 to FdU, a one week old culture of Fusarium venenatum WTY842-1-11 was prepared by plating a colonized agar plug of the strain taken from a 10% glycerol stock, which had been stored at −140° C., onto a VNO₃RLMT plate and incubating in a ChexAll Instant Seal Sterilization Pouch (Fisher Scientific, Pittsburgh, Pa., USA) for 7 days at 26-28° C. After 7 days plugs were cut sub-marginally from the one week old culture and placed face down on VNO₃RLMT medium supplemented with different concentrations of FdU (0 to 500 μM) (Sigma Chemical Co., St. Louis, Mo., USA) in 6 well plates. The plates were incubated at 26-28° C. in open ZIPLOC® bags (S.C. Johnson Home Storage, Inc., Racine, Wis., USA) for 14 days, after which the extent of growth at each FdU concentration was recorded.

Fusarium venenatum WTY842-1-11 was found to be insensitive to all FdU concentrations tested, although at concentrations greater than 100 μM, growth was slightly reduced compared to concentrations of 50 μM and below.

### Example 2

### Construction of Plasmid pJaL574

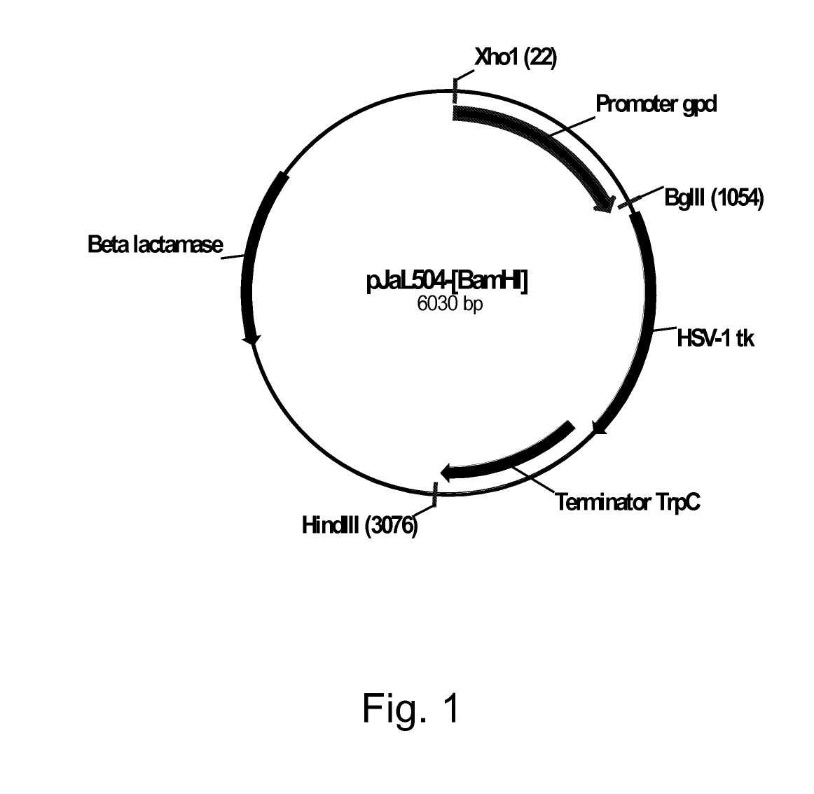

Plasmid pDV8 (U.S. Pat. No. 6,806,062) harbors the Herpes simplex virus type 1 thymidine kinase (HSV1-TK; tk) gene (SEQ ID NO: 37 for the DNA sequence and SEQ ID NO: 38 for the deduced amino acid sequence) as a 1.2 kb Bgl II/Bam HI fragment inserted between a 1.0 kb Xho I/Bgl II fragment of the Aspergillus nidulans glyceraldehyde-3-phosphate dehydrogenase (gpdA) promoter and a 1.8 kb Bam HI/Hind III fragment harboring the tri-functional Aspergillus nidulans indoleglycerolphosphate synthase, phosphoribosylanthranilate isomerase, and glutamine amidotransferase (trpC) transcriptional terminator. Plasmid pDV8 was digested with Bam HI, extracted with phenol-chloroform, ethanol precipitated, and then filled in using Klenow polymerase (Stratagene, La Jolla, Calif., USA). The digested plasmid was re-ligated using a QUICK LIGATION™ Kit (Roche Diagnostics Corporation, Indianapolis, Ind., USA) following the manufacturer's protocol, treated with a MINELUTE® Gel Extraction Kit (QIAGEN Inc., Valencia, Calif., USA), and the resulting ligation products cloned into pCR®4Blunt-TOPO® (Invitrogen, Carlsbad, Calif., USA) using a TOPO® Blunt Cloning Kit (Invitrogen, Carlsbad, Calif., USA) according to the manufacturer's instructions. The cloning reaction was transformed into ONE SHOT® chemically competent TOP10 cells (Invitrogen, Carlsbad, Calif., USA) according to the manufacturer's directions. Plasmid DNA was extracted from eight of the resulting transformants using a BIOROBOT® 9600 (QIAGEN Inc, Valencia, Calif., USA) and screened by restriction digestion using Xho I/Bam HI and Xho I/Hind III. DNA sequencing of plasmid DNA from two transformants with the correct restriction digestion pattern confirmed that both harbored the desired sequence. One was designated pJaL504-[Bam HI] (FIG. 1).

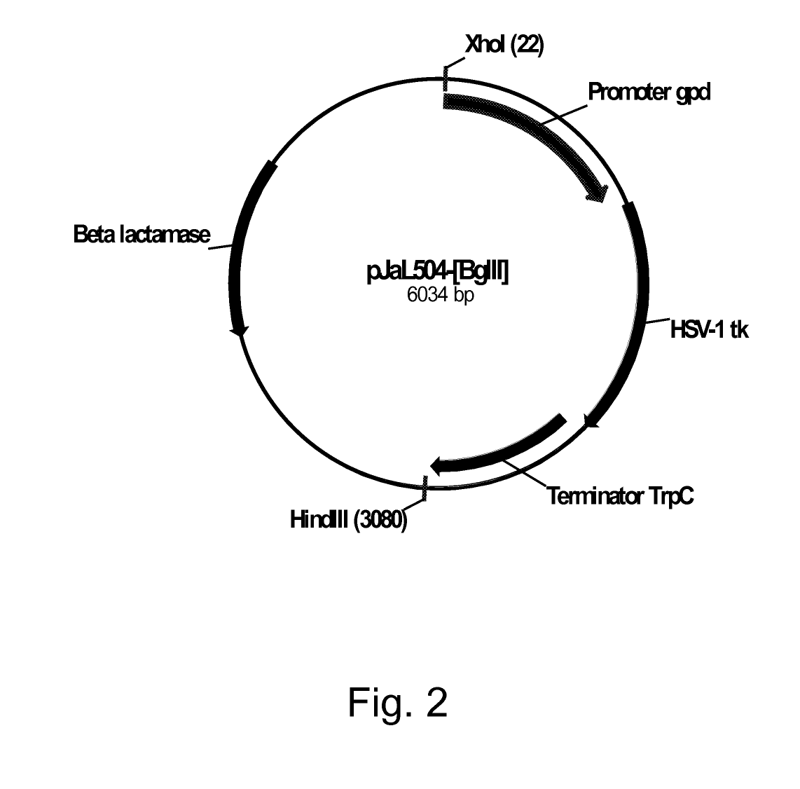

Plasmid pJaL504-[Bam HI] was digested with Bgl II, extracted with phenol-chloroform, ethanol precipitated, and then filled in using Klenow polymerase. The digested plasmid was re-ligated using a QUICK LIGATION™ Kit following the manufacturer's protocol, treated with a MINELUTE® Reaction Cleanup Kit, and the resulting ligation cloned into pCR®4Blunt-TOPO® using a TOPO® Blunt Cloning Kit according to the manufacturer's instructions. The cloning reaction was transformed into ONE SHOT® chemically competent TOP10 cells according to the manufacturer's directions. Plasmid DNA was extracted from eight of the resulting transformants using a BIOROBOT® 9600 and screened by restriction digestion using Xho I/Bgl II and Xho I/Hind III. DNA sequencing of plasmid DNA from two transformants with the correct restriction digestion pattern confirmed that both harbored the desired sequence. One was named pJaL504-[Bgl II] (FIG. 2). Punt et al. (1990, Gene 3: 101-109) have previously shown that 364 bp of the A. nidulans gpdA promoter could be deleted without affecting the strength of the promoter. Based on these authors' observations, primer #172450 shown below was designed to truncate the A. nidulans gpdA promoter and reduce the size of the vector.

Primer 172450:
(SEQ ID NO: 1)
5′-GACGAATTCTCTAGAAGATCTCTCGAGGAGCTCAAGCTTCTGTACA
GTGACCGGTGACTC-3′

The underlined sequence corresponds to gpdA promoter sequence. The remaining sequence is a handle harboring the following restriction sites: Eco RI, Xba I, Bgl II, Xho I, and Hind III.

For truncating the Aspergillus nidulans trpC terminator (again to reduce vector size), primer #172499, shown below, was designed harboring an Eco RI handle.

Primer 172499:
(SEQ ID NO: 2)
5′-GACGAATTCCGATGAATGTGTGTCCTG-3′

The underlined sequence corresponds to the trpC terminator sequence. Amplification using primers 172499 and 172450 truncates the promoter by 364 bp and the trpC terminator sequence by 239 bp.

PCR was performed with the above two primers using pJaL504-[Bgl II] as template to generate a 2.522 kb fragment composed of a truncated version of the A. nidulans gpdA promoter, the coding sequence of the HSV1-TK gene, and a truncated version of the A. nidulans trpC terminator.

The amplification reaction consisted of 5 μl of 10× Buffer (Promega Corporation, Madison, Wis., USA), 0.4 μl of 25 mM dNTPs, 1.25 μl of primer 172450 (100 ng/μl), 1.25 μl of primer 172499 (100 ng/μl), 0.5 μl of pJaL504-[Bgl II] (100 ng/μl), 2 μl of Pfu DNA polymerase (Promega Corporation, Madison, Wis., USA) (2.5 U/μl), and 39.6 μl of sterile distilled water. The amplification reaction was incubated in a ROBOCYCLER® (Stratagene, La Jolla, Calif., USA) programmed for 1 cycle at 95° C. for 45 seconds; and 28 cycles each at 95° C. for 45 seconds, 57° C. for 45 seconds, and 72° C. for 5 minutes. A final extension was performed for 10 minutes at 72° C.

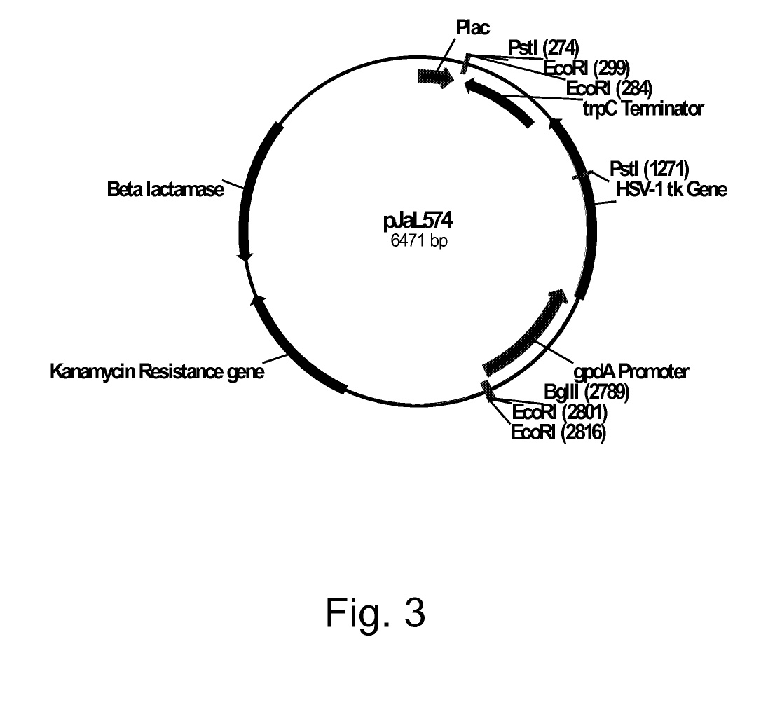

The amplification reaction was subjected to 1% agarose gel electrophoresis using low melting temperature agarose gel in 50 mM Tris-50 mM boric acid-1 mM disodium EDTA (TBE) buffer. A 2522 bp fragment was excised from the gel and extracted using a QIAQUICK® Gel Extraction Kit (QIAGEN Inc., Valencia, Calif., USA). The gel-purified DNA was then inserted into pCR®4Blunt-TOPO® using a TOPO® Blunt Cloning Kit according to the manufacturer's instructions. The cloning reaction was transformed into ONE SHOT® chemically competent TOP10 cells according to the manufacturer's directions. Plasmid DNA was extracted from eight of the resulting transformants using a BIOROBOT® 9600 and screened by restriction digestion using Eco RI and Bgl II. DNA sequencing of plasmid DNA from two transformants with the correct restriction digestion pattern confirmed that both harbored the desired sequence. One was designated pJaL574 (FIG. 3).

### Example 3

### Construction of Plasmid pWTY1449-02-01

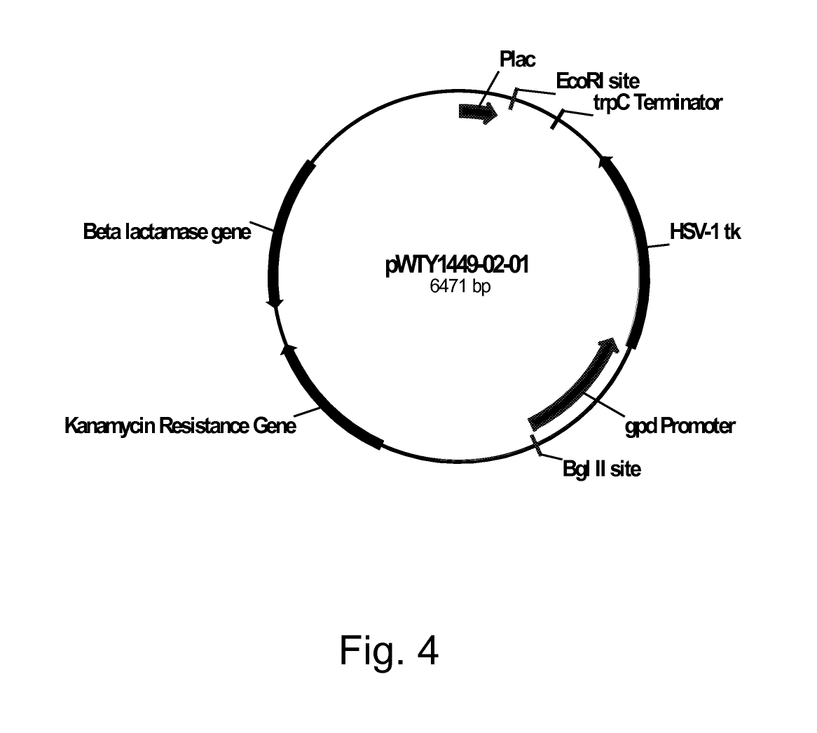

Plasmid pJaL574 was transformed into competent E. coli SCS110 cells (Stratagene, La Jolla, Calif., USA) following the manufacturer's recommended protocol. Plasmid DNA was extracted from twenty-four of the resulting transformants, using a BIOROBOT® 9600, and then subjected to analytical digestion using Eco RI and Bgl II. Subsequent DNA sequence analysis resulted in the identification of a clone with the correct sequence, which was designated pWTY1449-02-01 (FIG. 4).

### Example 4

### Construction of Plasmid pEJG61

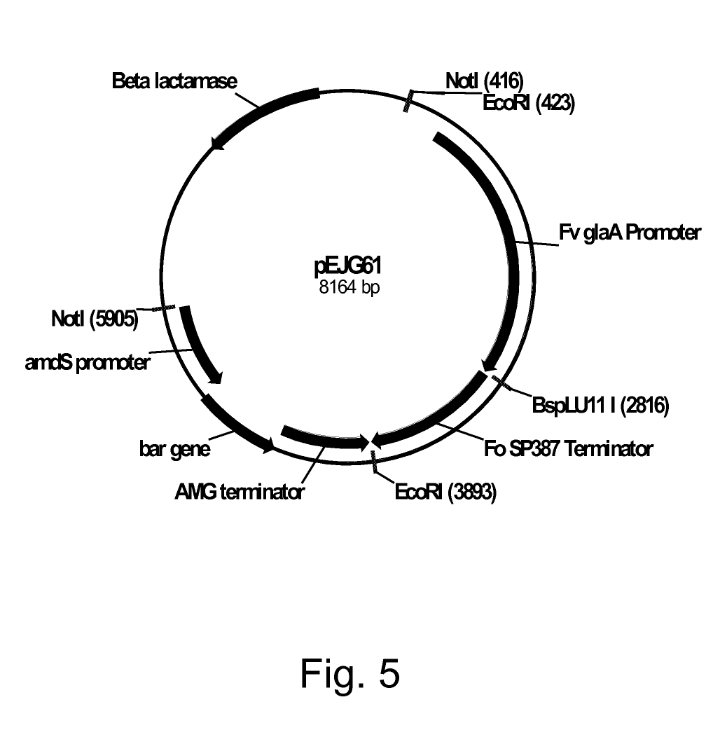

Plasmid pEJG61 (FIG. 5) was constructed as described in U.S. Pat. No. 7,368,271, with the exception that the orientation of the bar cassette was reversed (i.e., nucleotides 5901-5210 encode the amdS promoter, nucleotides 5209-4661 encode the bar coding sequence, and nucleotides 4660-4110 encode the Aspergillus niger glucoamylase (AMG) terminator).

### Example 5

### Generation of Spores and Protoplasts of Fusarium venenatum WTY842-01-11

To generate spores of Fusarium venenatum WTY842-01-11, 16 agar plugs (approximately 1 cm×1 cm), taken from a fresh agar culture (approximately 1 week old) as described in Example 1, were inoculated into 500 ml of RA medium in a 2.8 L Fernbach flask, and incubated at 26.5° C. for 24 hours with shaking at 150 rpm followed by an additional 12 hours at 28.5° C. The culture was then filtered through sterile MIRACLOTH™ (CalBiochem, San Diego, Calif., USA) in a sterile plastic funnel into the base of a 1 liter filtration unit through a 0.45 μM filter. The spores collected on the filter were washed with 500 ml of sterile distilled water and then resuspended in 10 ml of sterile distilled water and counted using a hemocytometer. The concentration was adjusted to 2×10⁸/ml.

The freshly generated spores were used to inoculate four 500 ml baffled shake flasks, each containing 100 ml of YPG_{5%} medium with 1 ml of fresh spores (2×10⁸/ml). The shake flasks were incubated at 23.5° C. for 15 hours with shaking at 150 rpm, by which time the germlings were approximately 3-5 spore lengths long. Twenty ml of 5 mg of NOVOZYME™ 234 per ml (Novozymes A/S, Bagsvaerd, Denmark), which had been filter sterilized in 1 M MgSO₄, were aliquoted into eight sterile 50 ml tubes. The germlings were then filtered through sterile MIRACLOTH™ in a sterile funnel and rinsed with 100 ml of sterile distilled water followed by 100 ml of sterile 1 M MgSO₄. Using a sterile spatula the rinsed germlings were scraped gently into the tubes containing the NOVOZYME™ 234 in 1 M MgSO₄ and mixed gently. The tubes were incubated on their sides, wedged in clamps, at 29° C. with shaking at 90 rpm for up to 1 hour. Thirty ml of 1 M sorbitol were added to each tube and the tubes were centrifuged at 377×g in a Sorvall RT 6000B swinging-bucket centrifuge (Thermo-Fischer Scientific, Waltham, Mass., USA) for 10 minutes at room temperature (approximately 24-28° C.). After decanting off the supernatant the pellets were gently resuspended in 1 ml of 1 M sorbitol. Thirty ml of 1 M sorbitol were then added and the tubes inverted gently several times. They were centrifuged at 377×g for 5 minutes at room temperature and the pellets gently resuspended in 1 ml of 1 M sorbitol. After gentle inversion of the tubes several times 30 ml of 1 M sorbitol were added and the tubes mixed gently. At this point a 100 μl aliquot was removed from each tube and added to an EPPENDORF® tube containing 900 μl of STC, for calculation of the protoplast concentration. The remaining suspensions were centrifuged for 5 minutes at 377×g at room temperature (approximately 24-28° C.). The supernatants were removed and the pellets resuspended in STC: SPTC:DMSO (9:1:0.1) so that the final concentration of protoplasts was 5×10⁷ per ml. The protoplasts were used immediately for co-transformation.

### Example 6

### Co-Transformation of pEJG61 and pWTY1449-01-02 into Fusarium venenaturn WTY842-01-11

Two ml of freshly generated Fusarium venenatum WTY842-01-11 protoplasts (5×10⁷/ml) were added to a 50 ml sterile centrifuge tube along with 50 μg each of circular pEJG61 and pWTY1449-02-01 in a volume of 80 μl (40 μl each). The protoplasts and DNA were mixed gently and then incubated on ice for 30 minutes. One hundred μl of SPTC were added slowly and gently mixed. The tube was incubated for 10 minutes at room temperature (26° C.). Eight ml of SPTC were added slowly and mixed by swirling gently. The tube was then incubated for 10 minutes at room temperature (26° C.). The reaction was then split between ten sterile 50 ml tubes (1 ml/tube). Thirty-five ml of VNO₃RLMT medium (top agarose) were then added to one tube at a time and mixed by inverting gently three times. The contents of each tube were then poured over pre-poured plates containing 35 ml of VNO₃RLMT medium supplemented with 12 mg of BASTA™ per ml. The plates were stored in ChexAll Instant Seal Sterilization Pouches for 3-4 days and then transferred to plastic bags for an additional 7-8 days. Colonies arising on the plates were sub-cultured to VNO₃RLMT-BASTA™ plates. Putative transformants were designated Fusarium venenatum WTY1449-03-01 through 29.

### Example 7

### Phenotypic Analysis of BASTA™-Resistant Transformants

Fusarium venenatum transformants WTY1449-03-01 through 29 were screened on three additional media: (1) VNO₃RLMT medium supplemented with different concentrations of FdU (0-500 μM); (2) VNO₃RLMT-BASTA™ and (3) VNO₃RLMT-BASTA™-FdU (the latter supplemented with FdU at 0 to 500 μM). The plates were incubated in opened plastic bags at ambient temperature (approximately 26° C.) for up to 15 days. Forty percent of the putative transformants were co-transformants (phenotypically), i.e., were able to grow on VNO₃RLMT-BASTA™ but not on VNO₃RLMT medium supplemented with different concentrations of FdU or VNO₃RLMT-BASTA™ medium supplemented with different concentrations of FdU.

### Example 8

### Genotypic Analysis of Putative bar+, tk+Co-Transformants

For five phenotypic bar+, tk+ co-transformants (Example 7), four small plugs were cut from seven day old cultures (described in Example 1) grown on VNO₃RLMT+BASTA™ medium and inoculated into baffled 125 ml shake flasks containing 25 ml of M400 medium to generate biomass for DNA extractions. The shake flasks were incubated at 28° C. for 4 days with shaking at 150 rpm. Biomass was then harvested through sterile MIRACLOTH™. The biomass was rinsed thoroughly with 200 ml of sterile distilled water, squeezed using gloved hands, and immersed in liquid nitrogen, using clean, long tweezers. Frozen biomass was either processed immediately or stored temporarily in sterile 50 ml plastic tubes at −80° C. After grinding the biomass in a mortar and pestle, genomic DNA was extracted using a DNEASY® Plant Maxi Kit (QIAGEN Inc., Valencia, Calif., USA), according to the manufacturer's directions except that the initial lytic incubation was extended to 90 minutes (from the 10 minutes suggested by the manufacturer). DNA was quantified using a NANODROP® ND-1000 Spectrophotometer (Thermo Scientific, Wilmington, Del., USA). Aliquots from each stock containing 8 μg of DNA were then concentrated to dryness, using a SPEEDVAC® Concentrator (Thermo-Electron Corp., Waltham, Mass., USA), after which time 60 μl of 10 mM Tris pH 8.0 were added to each sample and mixed.

Eight micrograms of DNA from each strain were digested with Eco RI and selected strains were also digested with Bam HI. Eco RI reactions were composed of 1× Eco RI buffer, 8 μg of DNA, 65 units of Eco RI, and sterile distilled water to a final volume of 100 μl. After incubation at 37° C. for 10 hours, loading buffer (40% sucrose, 5 mM EDTA, 0.025% bromophenol blue, 0.025% xylene cyanol) was added and samples were loaded onto four 1% agarose gels, which were run in TBE buffer at 60 volts for 5 hours. Bam HI restriction digests were composed of 1×NEB buffer 3 (New England Biolabs Inc., Ipswich, Mass., USA), 8 μg of DNA, 65 units of Bam HI, 100 μg of bovine serum albumin per ml, and sterile distilled water to a final volume of 100 μl. After incubation at 37° C. for 10 hours, loading buffer was added, and samples were loaded onto 1% agarose gels, which were run in TBE buffer at 60 volts for 5 hours.

Following ethidium bromide staining and de-staining, Southern blots were prepared from the gels using HYBOND™ N nylon membranes (Amersham Biosciences, Buckinghamshire, UK) as follows. Depurination was conducted in 0.25 N HCl for 10 minutes at 26° C. with gentle shaking followed by a 5 minute wash in sterile distilled water at 26° C. The wash was followed by two denaturing reactions using 0.5 N NaOH/1.5 M NaCl at 26° C. for 15 minutes (1^{st} reaction) and 20 minutes (2^{nd} reaction) with gentle shaking. Another wash followed in sterile distilled water at 24-26° C. for 2 minutes with gentle shaking. The final wash was followed by two neutralization reactions using 1.5 M NaCl, 0.5 M Tris pH 7.5, and 0.001 M EDTA for 30 minutes each at 24-26° C. with gentle shaking. The membranes were then blotted overnight using a TURBO BLOTTER™ Kit (Schleicher & Schuell, Keene, N.H., USA) in 10×SSC at 24-26° C. The membranes were washed for 5 minutes in 2×SSC with shaking at 24-26° C. The membranes were then air-dried for 10 minutes at 24-26° C., UV-cross linked using a STRATALINKER™ (Stratagene, La Jolla, Calif., USA) (on the automatic setting which generates a total dose of 120 mJ/cm²), and finally baked in a vacuum oven at 80° C. for 1 hour.

Primers, shown below, for generating bar- and tk gene-specific probes were designed using Vector NTI® software (Invitrogen, Carlsbad, Calif., USA).

bar Gene Forward Primer # 996023:
(SEQ ID NO: 3)
5′-CGAGTGTAAACTGGGAGTTG-3′
bar Gene Reverse Primer # 996024:
(SEQ ID NO: 4)
5′-GAGCAAGCCCAGATGAGAAC-3′
tk Gene Forward Primer # 998744:
(SEQ ID NO: 5)
5′-GGCGATTGGTCGTAATCCAG-3′
tk Gene Reverse Primer # 998745:
(SEQ ID NO: 6)
5′-TCTTCGACCGCCATCCCATC-3″

DIG-labeled probes of the bar and tk genes were generated using a PCR DIG Probe Synthesis Kit (Roche Diagnostics Corporation, Indianapolis, Ind., USA) according to the manufacturer's protocol. After cycling, the reactions were placed on ice, centrifuged momentarily in a microfuge, and then loaded onto 1% agarose gels. After electrophoresis in TBE buffer, bands of the predicted size were excised and gel purified using a MINELUTE® Gel Extraction Kit.

Filters were pre-hybridized in 35 ml of DIG Easy Hyb (Roche Diagnostics Corporation, Indianapolis, Ind., USA) in glass tubes for 3 hours at 42° C. after which the DIG Easy Hyb was removed and replaced with 7.5 ml of fresh DIG Easy Hyb plus 10 μl of labeled probe, which had been boiled for five minutes and then placed on ice (i.e., approximately 30% of the gel-purified DNA which resulted from the PCR reaction was used). Hybridizations were performed at 42° C. in a hybridization oven for 12 hours. Two 5 minute post-hybridization washes were performed at room temperature in 2×SSC, 0.1% SDS, followed by two 15 minute washes at 65° C. in 0.2×SSC, 0.1% SDS. Subsequent washing and detection was conducted using a DIG Wash and Block Set, Anti-Digoxigenin-AP Fab Fragments, and CDP-Star Chemi-luminescent substrate (Roche Diagnostics Corporation, Indianapolis, Ind., USA), as recommended by the manufacturer. One of the Fusarium venenatum strains confirmed as being a true bar+, tk+ co-transformant by phenotypic (Example 7) and Southern analyses (this example) was designated Fusarium venenatum WTY1449-03-03.

### Example 9

### Curing the tk Gene from Fusarium venenatum WTY1449-03-03 bar+, tk+ Co-Transformant

Sporulation of Fusarium venenatum strain WTY1449-03-03 was induced, as described in Example 5, in RA+BASTA™ medium. The spores were subsequently screened for growth on FdU-supplemented media, which should induce loss of the tk gene. From inoculation of 25 ml of RA medium with four plugs cut from a fresh culture of this strain, 1.06×10⁸ spores were obtained. This spore stock was used to make a series of dilutions for plating to both 15 mm diameter FdU-supplemented VNO₃RLMT plates and unsupplemented VNO₃RLMT plates (the latter for viability estimates). Spores (100 to 1×10⁷) were spread on duplicate plates and incubated at approximately 26° C. in ChexAll Instant Seal Sterilization Pouches for 5 days.

Southern analysis (using the bar and tk probes described in Example 8) was performed on five selected colonies, which were able to grow when sub-cultured onto 25 μM FdU, using the procedure described in Example 7. The results revealed that all five of the single spore isolates had been cured of the tk gene. One strains was designated Fusarium venenatum WTY1449-09-01.

### Example 10

### Confirmation that Uridine Supplementation Negates the FdU-Sensitive Phenotype of tk-Harboring Transformants

It was important to determine whether supplementation of growth media with uridine interfered with the mechanism of FdU-sensitivity of tk⁺ strains, in order to optimize the gene deletion system for use in pyrG-deleted strains, which require uridine supplementation for viability.

To this end the bar+, tk+ strain Fusarium venenatum WTY1449-03-03 was revived on VNO₃RLMT-BASTA™ plates (as described in Example 1) and induced to produce spores as described in Example 5. After harvesting and washing, the spores were plated (50,000 spores per 14 cm diameter plate) to FdU-supplemented VNO₃RLMT plates containing 50 μM FdU and varying concentrations of uridine (0.1-1 mM). These plates were incubated at 28° C. in ChexAll Instant Seal Sterilization Pouches for 6 days, after which they were evaluated for growth.

While no growth was observed on FdU-supplemented VNO₃RLMT plates without uridine, extensive growth of the tk+ strain occurred at all concentrations (0.1-1 mM) of uridine- and FdU-supplemented VNO₃RLMT. This situation makes it difficult or impossible to distinguish tk+ from tk− strains on FdU-containing media. As a result it was necessary to optimize the uridine and FdU concentrations to determine if there was any combination that would allow tk+ and tk− strains to be distinguished on FdU- and uridine-supplemented media (Examples 15 and 16).

### Example 11

### Generation of pEmY21

An E. coli hygromycin phosphotransferase (hpt) gene (SEQ ID NO: 7 for the DNA sequence and SEQ ID NO: 8 for the deduced amino acid sequence) was amplified from plasmid pPHTI (Cummings et al., 1999, Current Genetics 36: 371-382) using the following primers:

Forward primer:
(SEQ ID NO: 9)
5′-GGGttcgaaTTCATTTAAACGGCT-3′
Reverse primer:
(SEQ ID NO: 10)
5′-GGGagcgctCAATATTCATCTCTC-3′

The restriction enzyme sites Bst BI (forward primer) and Eco 47III (reverse primer) were engineered into the primers, represented by the underlined sequences, for cloning.

The PCR reaction (to amplify the hpt gene) was composed of 1× ThermoPol Buffer (New England Biolabs, Ipswich, Mass., USA), 200 μM dNTPs, 50 pmol of the forward and reverse primers, 100 pg of pPHT1, 1 unit of Vent® DNA polymerase (New England Biolabs Inc., Ipswich, Mass. USA), and sterile distilled water in a total volume of 100 μl. The amplification reaction was performed using a ROBOCYCLER® programmed for 1 cycle at 95° C. for 2 minutes; 25 cycles each at 95° C. for 1 minute, 51° C. for 1 minute, and 72° C. for 2 minutes; and 1 cycle at 72° C. for 7 minutes.

PCR products were separated by 1% agarose gel electrophoresis in 40 mM Tris base-20 mM sodium acetate-1 mM disodium EDTA (TAE) buffer. A 1.8 kb fragment was excised from the gel and agarose extracted using a QIAQUICK® Gel Extraction Kit. The gel purified fragment was then cloned into pCR®-Bluntll-TOPO® (Invitrogen, Carlsbad, Calif., USA) using a TOPO® Blunt Cloning Kit. The resulting plasmid was designated pEmY10.

The Eco RI site was then removed from the coding sequence of the hpt gene in pEmY10 using a QUIKCHANGE® Site-Directed Mutagenesis Kit (Stratagene, La Jolla, Calif., USA) according to the manufacturer's instructions using the primers shown below, where the lower case letters represent the non-mutated nucleotides of the target Eco RI site and the underlined case letters represent the mutated nucleotides. The resulting plasmid was designated pBK3.

Forward primer:
(SEQ ID NO: 11)
5′-GGGTACCCCAAGGGCgTattcTGCAGATGGG-3′
Reverse primer:
(SEQ ID NO: 12)
5′-CCCATCTGCAgaatAcGCCCTTGGGGTACCC-3′

The resulting hpt gene without the Eco RI site was PCR amplified from pBK3 using forward and reverse primers shown below.

Forward primer:
(SEQ ID NO: 13)
5′-GGggtaccTTCATTTAAACGGCTTCAC-3′
Reverse primer:
(SEQ ID NO: 14)
5′-GGggtaccCGACCAGCAGACGGCCC-3′

The underlined portions represent introduced Kpn I sites for cloning.

Portions of the Aspergillus oryzae pyrG gene were used to generate direct repeats and were PCR amplified from pSO₂ (WO 98/12300) using the following primers:

Repeat 1:
Forward primer:
(SEQ ID NO: 15)
5′-TCCcccgggTCTCTGGTACTCTTCGATC-3′
Reverse primer:
(SEQ ID NO: 16)
5′-GGggtaccCGACCAGCAGACGGCCC-3′
Repeat 2:
Forward primer:
(SEQ ID NO: 17)
5′-GGggtaccTCTCTGGTACTCTTCGATC-3′
Reverse primer:
(SEQ ID NO: 18)
5′-TCCcccgggCGACCAGCAGACGGCCC-3′

The underlined portions represent introduced restriction sites Sma I (cccggg) or Kpn I (ggtacc) for cloning.

The three fragments (hpt, repeat #1 and repeat #2) were amplified in separate reactions (50 μl each) composed of 1× ThermoPol Buffer, 200 μM dNTPs, 0.25 μM each primer, 50 ng of template DNA, and 1 unit of Vent® DNA polymerase. The amplification reaction was performed using a ROBOCYCLER® programmed for 1 cycle at 95° C. for 2 minutes; 30 cycles each at 95° C. for 1 minute, 61° C. for 1 minute, and 72° C. for 2 minutes; and 1 cycle at 72° C. for 7 minutes.

The PCR products were separated by 1.5% agarose gel electrophoresis in TAE buffer. The approximately 2 kb amplified hpt fragment and the approximately 0.2 kb repeat fragments were excised from the gels and agarose-extracted using a MINELUTE® Gel Extraction Kit. The two pyrG repeat fragments were digested with Kpn I, dephosphorylated with calf intestine phosphatase (New England Biolabs Inc., Ipswich, Mass., USA), and treated with a MINELUTE® Reaction Cleanup Kit (QIAGEN Inc., Valencia, Calif., USA) according to the manufacturer's instructions. The fragments harboring repeat #1 and hpt were then ligated together using a QUICK LIGATION™ Kit according to the manufacturer's instructions, treated with a MINELUTE® Reaction Cleanup Kit, and cloned into pCR®-BluntII-TOPO® using a TOPO® Blunt Cloning Kit. Sequence analysis confirmed one clone in which repeat #1 and the hpt fragment were ligated together. This plasmid was designated pEmY18.

In order to clone the second repeat into pEmY18, plasmid pEmy18 was digested with Eco RV and the digestion purified by 1% agarose gel electrophoresis in TAE buffer. A 5.6 kb fragment was excised from the gel and agarose-extracted using a QIAQUICK® Gel Extraction Kit. The 0.2 kb Repeat 2 fragment (described above) and digested pEmY18 were ligated together using a QUICK LIGATION™ Kit. The ligation mixture was used to transform SOLOPACK® Gold Supercompetent Cells (Stratagene, La Jolla, Calif., USA). Sequence analysis identified a plasmid in which the three components (repeat #1, hpt, and repeat #2) were in the desired order and orientation and lacked PCR errors. The resulting plasmid was designated pEmY20.

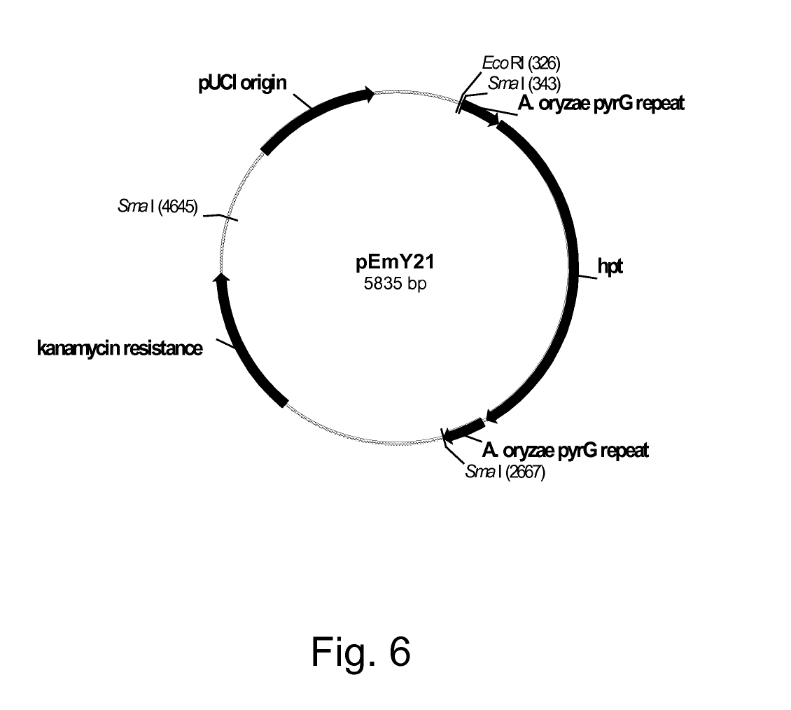

To insure that subsequent digestion of pEmY20 with Eco RI would liberate a single fragment, an Eco RI site was removed using a QUIKCHANGE® Site-Directed Mutagenesis Kit according to the manufacturer's instructions and forward and reverse primers shown below. The resulting plasmid was designated pEmY21 (FIG. 6) after sequence verification.

Forward primer:
(SEQ ID NO: 19)
5′-GGGTACCCCAAGGGCQTATTCTGCAGATGGG-3′
Reverse primer:
(SEQ ID NO: 20)
5′-CCCATCTGCAGAATACGCCCTTGGGGTACCC-3′

### Example 12

### Construction of Plasmid pDM156.2, Harboring the Genomic DNA Fragment Incorporating the Fusarium venenaturn Orotidine-5′-Monophosphate Decarboxylase (pyrG) Gene

A probe of a Neurospora crassa orotidine-5′-monophosphate decarboxylase (pyr-4) gene (SEQ ID NO: 21 for the DNA sequence and SEQ ID NO: 22 for the deduced amino acid sequence) was prepared by PCR incorporating digoxigenin-labeled deoxyuridine-triphosphate (dUTP) using the primers described below.

Primer (sense):
(SEQ ID NO: 23)
5′-GTCAGGAAACGCAGCCACAC-3′
Primer (anti-sense):
(SEQ ID NO: 24)
5′-AGGCAGCCCTTGGACGACAT-3′

Plasmid pFB6 (Buxton et al, 1983, Molecular and General Genetics 190: 403-405) was digested with Hind III and the digestion purified by 1% agarose gel electrophoresis using TAE buffer. A 1.1 kb pyr-4 fragment was excised and agarose-extracted using a QIAQUICK® Gel Extraction Kit according to the manufacturer's suggested protocols.

The amplification reaction (50 μl) was composed of 1× Taq DNA Polymerase Buffer (New England Biolabs Inc., Ipswich, Mass., USA), 5 μl of PCR DIG Labeling Mix (Boehringer Mannheim, Manheim, Germany), 10 ng of the 1.1 kb Hind III pyr-4 fragment, 10 pmol of the sense primer, 10 μmol of the anti-sense primer, and 1 unit of Taq DNA polymerase (New England Biolabs Inc., Ipswich, Mass., USA). The reaction was incubated in a ROBOCYCLER® programmed for 1 cycle at 95° C. for 3 minutes followed by 35 cycles each at 95° C. for 30 seconds, 55° C. for 1 minute, and 72° C. for 1 minute. A final extension was performed for 5 minutes at 72° C.

The amplification reaction products were purified by 1% agarose gel electrophoresis using TAE buffer. A digoxigenin (DIG) labeled probe of approximately 0.78 kb was excised from the gel and agarose-extracted using a QIAQUICK® Gel Extraction Kit.

A genomic DNA library of Fusarium venenatum strain A3/5 was generated and cloned into lambda vector EMBL4 as described in WO 99/60137.

The DIG-labeled probe was used to screen the genomic library of Fusarium venenatum A3/5 DNA cloned into lambda vector EMBL4. Lambda phage were plated with E. coli K802 cells (New England Biolabs, Ipswich, Mass., USA) onto LB plates with NZY top agarose. Plaque lifts were made to HYBOND™ N nylon membranes using the technique of Sambrook et al. (Molecular Cloning, A Laboratory Manual, Second Edition; J. Sambrook, E. F. Fritsch, and T. Maniatis; Cold Spring Harbor Laboratory Press, 1989). DNA was bound to the membranes by UV cross-linking using a UV STRATALINKER™. Filters were then hybridized with the 0.78 kb DIG-labeled N. crassa pyr-4 probe. Hybridization and detection of pyrG clones were performed according to the GENIUS™ System User's Guide (Boehringer Hammheim, Manheim, Germany) at 42° C. with a hybridization solution composed of 5×SSC, 35% formamide, 0.1% L-lauroylsarcosine, 0.02% SDS, and 1% blocking reagent (Boehringer Hammheim, Manheim, Germany). The concentration of DIG-labeled probe used was 2.5 ng per ml of the hybridization solution. Hybridizing DNA was immuno-detected with an alkaline-phosphatase-conjugated anti-digoxigenin antibody (Boehringer Hammheim, Manheim, Germany) and visualized with Lumiphos 530, a chemiluminescent substrate (Boehringer Hammheim, Manheim, Germany). DNA preparations were made from putative positive lambda clones using a Lambda Midi Kit (QIAGEN Inc., Valencia, Calif., USA).

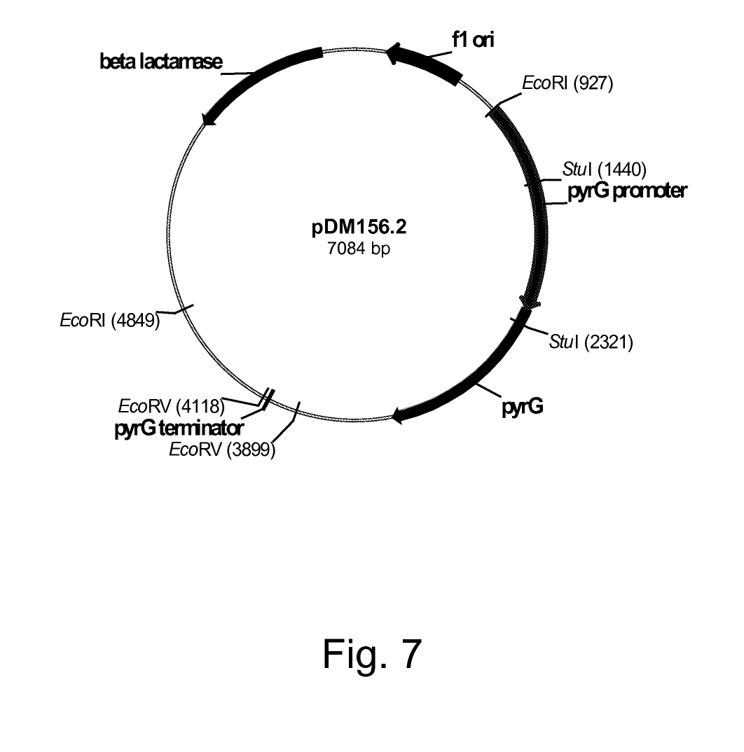

Lambda DNA from a clone identified above was digested with Eco RI and subjected to 1% agarose gel electrophoresis in TAE buffer. A 3.9 kb fragment was excised and agarose-extracted using a QIAEX Gel Extraction Kit (QIAGEN Inc., Valencia, Calif.). The fragment was then cloned into the Eco RI site of pUC118 (Viera and Messing, 1987, Methods in Enzymology 153: 3-11) and ONE SHOT® TOP10 competent cells were transformed with 2 μl of the cloning reaction. Plasmid DNA from eight of the resulting transformants was analyzed by DNA sequencing. One clone with the desired sequence was selected and designated pDM156.2 (FIG. 7). The pyrG fragment harbored the entire coding region plus 1.3 kb of the promoter and 1.5 kb of the terminator.

### Example 13

### Creation of the Fusarium venenatum pyrG Deletion Vector pEmY23

The Fusarium venenatum pyrG coding sequence (2,678 bp, SEQ ID NO: 51 for the DNA sequence and SEQ ID NO: 52 for the deduced amino acid sequence) was excised from pDM156.2 (Example 12) by digestion with Eco RV and Stu I, and the remaining 4,398 bp vector was gel-purified using a QIAQUICK® Gel Extraction Kit according to the manufacturer's directions. The Sma I fragment of pEmY21 was isolated and gel-purified using a QIAQUICK® Gel Extraction Kit and the two gel-purified fragments were ligated together using a QUICK LIGATION™ Kit according to the manufacturer's instructions and treated with a MINELUTE® Reaction Cleanup Kit, and 2 μl of the resulting ligation were used to transform ONE SHOT® chemically competent TOP10 cells according to the manufacturer's directions.

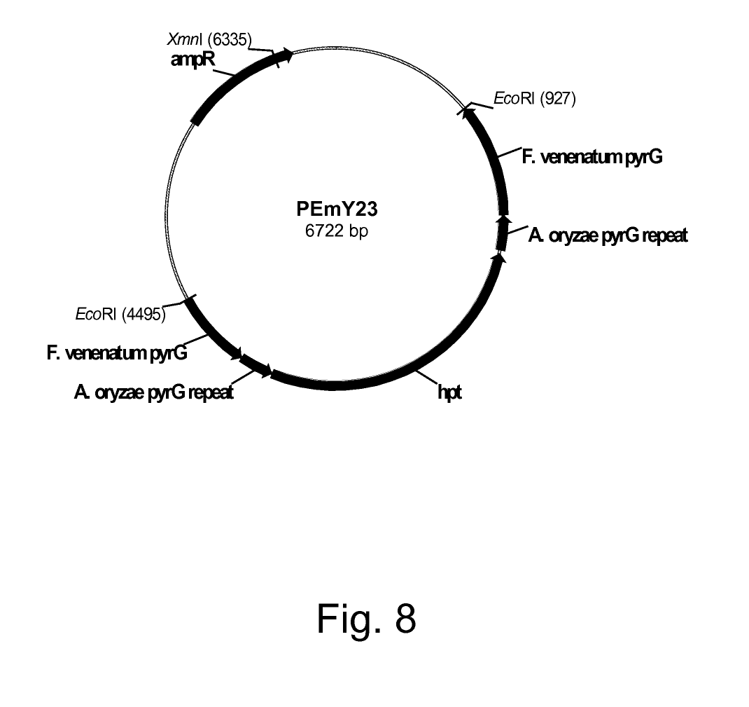

Plasmid DNA was extracted from eight of the resulting transformants using a BIOROBOT® 9600. These DNAs were screened for insert orientation, sequenced for the absence of errors, and one of the clones with the correct insert sequence was selected and designated pEmY23 (FIG. 8).

### Example 14

### Creation of the pyrG-Deleted Strain EmY1154-46-4.3

Plasmid pEmY23 was digested with Eco RI and Xmn I and subjected to 1% agarose gel electrophoresis in TAE buffer in order to isolate a 3.6 kb DNA fragment. The 3.6 kb fragment was gel-purified using a QIAQUICK® Gel Extraction Kit according to the manufacturer's directions and used to transform protoplasts of Fusarium venenatum WTY842-1-11 as described in Example 6, with two differences: Firstly, only one type of transforming DNA was used (the 3.6 kb Eco RI-Xmn I digested pEmY23 fragment), and secondly, transformants were selected on VNO₃RLMT supplemented with 1 mM uridine and 0.125 mg of hygromycin B (Roche, Indianapolis, Ind., USA) per ml. Ten transformants were chosen for screening in 25 ml of unsupplemented M400 liquid medium and also in a phenotypic screen on VNO₃RLMT+1 mM uridine (positive control for growth), VNO₃RLMT+1 mM uridine+0.125 mg per ml hygromycin B (positive control for transformation), and unsupplemented VNO₃RLMT (screen for pyrG deletion). Candidates for uridine prototrophy could be identified within three days in liquid medium and seven days by the plate-based phenotype screen. One of the candidates chosen for further screening and spore purification was designated EmY1154-46-4. Spore-purified isolates derived from this strain (obtained as described in Example 21, except that the agar medium was VNO₃RLMT supplemented with 10 mM uridine) were subjected to the same screening protocols described above and two single spore isolates were chosen for Southern hybridization analysis for comparison with the parent strain. These spore-purified strains were designated Fusarium venenatum EmY1154-46-4.3 and EmY1154-46-4.5.

Genomic DNA was prepared as described in Example 8 from Fusarium venenatum WTY842-1-11 (the control strain) for the presence of pyrG and absence of hpt, primary transformant Fusarium venenatum EmY1154-46-4, and single spore isolates Fusarium venenatum EmY1154-46-4.3 and EmY1154-46-4.5. Eight micrograms of DNA from each strain were digested with Stu I and Mfe I. Stu I reactions were composed of 1×NEB buffer 2 (New England Biolabs Inc., Ipswich, Mass., USA), 8 μg of DNA, 65 units of Stu I, and sterile distilled water to a total volume of 100 μl. After incubation at 37° C. for 10 hours, loading buffer (40% sucrose, 5 mM EDTA, 0.025% bromophenol blue, 0.025% xylene cyanol) was added and the samples were loaded onto two 1% agarose gels, which were run in TBE buffer at 60 volts for 5 hours. Mfe I restriction digests were composed of 1×NEB buffer 4 (New England Biolabs Inc., Ipswich, Mass., USA), 8 μg of DNA, 65 units of MFe I, and sterile distilled water to a total volume of 100 μl. After incubation at 37° C. for 10 hours, loading buffer was added, and samples were loaded onto 1% agarose gels, which were run in TBE buffer at 60 volts for 5 hours.

Following ethidium bromide staining and de-staining, Southern blots were prepared from the gels using HYBOND™ N nylon membranes as follows. Depurination was conducted in 0.25 N HCl for 10 minutes at 26° C. with gentle shaking followed by a 5 minute wash in sterile distilled water at 26° C. The wash was followed by two denaturing reactions using 0.5 N NaOH/1.5 M NaCl at 26° C. for 15 minutes (1^{st} reaction) and 20 minutes (2^{nd} reaction) with gentle shaking. Another wash followed in sterile distilled water at 26° C. for 2 minutes with gentle shaking. The final wash was followed by two neutralization reactions using 1.5 M NaCl, 0.5 M Tris pH 7.5, and 0.001 M EDTA for 30 minutes each at 26° C. with gentle shaking. The membranes were then blotted overnight using a TURBO BLOTTER™ Kit in 10×SSC at 26° C. The membranes were washed for 5 minutes in 2×SSC with shaking at 26° C. The membranes were then air-dried for 10 minutes at 26° C., UV-cross linked using a STRATALINKER™ (on the automatic setting which generates a total dose of 120 mJ/cm²), and finally baked in a vacuum oven at 80° C. for 1 hour.

Primers shown below for generating pyrG and hpt gene-specific probes were designed using Vector NTI® software (Invitrogen, Carlsbad, Calif., USA).

Fusarium venenatum pyrG forward primer:
(SEQ. ID NO: 25)
5′-GCCATGCGATCCAGCGTTTGAATCC-3′
Fusarium venenatum pyrG forward primer:
(SEQ. ID NO: 26)
5′-GCGTCCGCAACTGACGATGGTCCTC-3′
E. coli hpt forward primer:
(SEQ. ID NO: 27)
5′-CAGATACCACAGACGGCAAGC-3′
E. coli hpt reverse primer:
(SEQ. ID NO: 28)
5′-GGGCAGTTCGGTTTCAGG-3′

DIG-labeled probes of the pyrG and hpt genes were generated using a PCR DIG Probe Synthesis Kit according to the manufacturer's protocol. After cycling, the reactions were placed on ice, centrifuged momentarily in a microfuge, and then loaded onto 1% agarose gels. Following electrophoresis in TBE buffer, bands of the predicted size were excised and gel-purified using a MINELUTE® Gel Extraction Kit. Filters were pre-hybridized in 35 ml of DIG Easy Hyb in glass tubes for 3 hours at 42° C. after which time the Easy Hyb was removed and replaced with 7.5 ml of fresh DIG Easy Hyb plus 10 μl of labeled probe (approximately 30% of the gel-purified DNA amplified from the PCR reactions), which had been boiled for five minutes and then placed on ice. Hybridizations were performed at 42° C. in a hybridization oven for 12 hours. Two 5 minute post-hybridization washes were performed at room temperature in 2×SSC, 0.1% SDS, followed by two 15 minute washes at 65° C. in 0.2×SSC, 0.1% SDS. Subsequent washing and detection was conducted using DIG Wash and Block Set, Anti-Digoxigenin-AP Fab Fragments, and CDP-Star Chemi-luminescent substrate, as recommended by the manufacturer.

Southern hybridization results revealed that Fusarium venenatum EmY1154-46-4 and its two single spore isolates EmY1154-46-4.3 and EmY1154-46-4.5 had sustained pyrG deletion events and harbored the hpt gene.

### Example 15

### Germination Efficiency of Spores of the pyrG-Deleted Fusarium Venenatum Strain EmY1154-46-4.3 on Uridine- and FdU-Supplemented Media

Germination efficiency of spores from the pyrG-deleted Fusarium venenatum strain EmY1154-46-4.3 on uridine- and FdU-supplemented media was tested. Spores of Fusarium venenatum EmY1154-46-4.3 were generated as described in Example 5 using RA medium supplemented with 10 mM uridine. Fifty spores in a volume of 200 μl were aliquoted to each of 45 VNO₃RLMT plates (14 cm diameter) supplemented with 0, 25, or 50 μM FdU and 0, 0.01, 0.05, 0.1, or 0.25 mM uridine. Triplicate plates of each combination of FdU and uridine were set up and incubated in ChexAll Instant Seal Sterilization Pouches at 26° C. for 10 days.

At a uridine concentration of 0.01 mM, spores of Fusarium venenatum EmY1154-46-4.3 could not germinate in the presence of 25 or 50 μM FdU, while they germinated readily in the absence of FdU on the same medium. However, at uridine concentrations of 0.1 mM, spores of the pyrG-deleted strain could germinate at a frequency of approximately 50% in the presence of 25 and 50 μM FdU (compared with a frequency of 75% in the absence of FdU).

### Example 16

### Distinction of tk+ and tk− Strains on FdU-Supplemented Minimal Media at Low Uridine Concentrations

To determine whether very low uridine concentrations could confer resistance to FdU in a tk+ strain, a reconstruction experiment was performed. The tk+ strain Fusarium venenatum WTY1449-3-3 and the tk− strain Fusarium venenatum WTY1449-9-1 were used. Spores of each strain were induced and plated at 50 spores per plate (Fusarium venenatum WTY1449-9-1) or 50,000 spores per 14 cm diameter plate (Fusarium venenatum WTY1449-3-3). In addition, a combination of WTY1449-3-3 and WTY1449-9-1 spores, 50 and 50,000 respectively, were mixed and plated. All plates contained VNO₃RLMT supplemented with 50 μM FdU. The uridine concentration in the plates was 1, 0.5, 0.25, or 0.1 mM. Each treatment was performed in triplicate.

The tk+ strain grew as a uniform haze on all plates, except on the medium lacking uridine, on which it did not grow. The tk− strain grew well at all concentrations of uridine and on the medium lacking uridine. On the mixed plates the results were a combination of the results from the pure plates of the tk+ and tk− strains. On each uridine-containing plate the distinct tk− colonies were superimposed over the hazy background growth of the tk+ strain.

Colonies appearing on the plates on which mixtures of tk+ and tk− spores had been plated were sub-cultured to fresh plates of VNO₃RLMT medium supplemented with 50 μM FdU (NO uridine). An equivalent number of samples were also sub-cultured from the background growth (3 colonies per mixed plate) to VNO₃RLMT+50 μM FdU (No uridine). In addition, colonies and background growth were sub-cultured from pure tk− plates and pure tk+ plates to plates of VNO₃RLMT+50 μM FdU (no uridine). This was done to evaluate whether (1) the background growth on mixed plates (presumptive FdU-sensitive, tk+ strains) would subsequently manifest the expected phenotype (sensitivity to FdU) in the absence of uridine; and (2) the presumptive FdU-resistant, tk− colonies would grow normally under these conditions. After incubation it became apparent that the tk+ strains could absolutely not grow on uridine-deficient media in the presence of 50 μM FdU, while the tk− strains grew normally on uridine-deficient media in the presence of 50 μM FdU. Despite the background haze of tk+ growth on mixed plates with uridine, the tk− strains were easily distinguishable and could be easily sub-cultured from FdU-containing media supplemented with 0.1 mM uridine to uridine-free media, without the danger of being contaminated with tk+ strains, as is required with the claimed dual selection technology.

The results demonstrated that the tk gene can be employed successfully as a negatively selectable marker under uridine-supplemented growth conditions (contrary to published statements that supplementation with uridine abrogates the inhibitory effects of FdU, e.g., Sachs et al., 1997, Nucleic Acids Research 25: 2389-2395).

### Example 17

### Construction of Plasmid pWTY1470-19-07

Plasmid pJRoy40 (U.S. Pat. No. 7,332,341), which harbors 5′ and 3′ flanking sequences of a Fusarium venenatum trichodiene synthase (tri5) gene (SEQ ID NO: 29 for the DNA sequence and SEQ ID NO: 30 for the deduced amino acid sequence), was used as template for amplification of a portion of the 5′ tri5 gene flanking sequence. The PCR reaction contained 200 μM dNTPs, 1× Taq DNA polymerase buffer, 125 pg of pJRoy40 DNA, 50 pmol of each primer shown below, and 1 unit of Taq DNA polymerase in a final volume of 50 μl.

Forward primer:
(SEQ ID NO: 31)
5′-GGGAGATCTTCGTTATCTGTGCC-3′
Reverse primer:
(SEQ ID NO: 32)
5′-GGGAGATCTTAGTAGTCGGCATTTGAAAC-3′
(Underlined nucleotides indicate
introduced Bgl II sites).

The amplification reaction was incubated in a ROBOCYCLER® programmed for 1 cycle at 95° C. for 3 minutes; 10 cycles each at 95° C. for 30 seconds, 52° C. for 45 seconds, and 7° C. for 2 minutes; 20 cycles each at 95° C. for 30 seconds, 52° C. for 45 seconds, and 72° C. for 5 minutes; and 1 cycle at 72° C. for 7 minutes.

PCR products were separated by 1.5% agarose gel electrophoresis using TBE buffer. A fragment of approximately 600 bp was excised from the gel and agarose-extracted using a MINELUTE® Gel Extraction Kit. The fragment was inserted into pCR®2.1 (Invitrogen, Carlsbad, Calif., USA) using a TOPO® TA Cloning Kit (Invitrogen, Carlsbad, Calif., USA) and ONE SHOT® TOP10 competent cells were transformed with 2 μl of the cloning reaction. Plasmid DNA from eight of the resulting transformants was digested with Eco RI and Bgl II in separate reactions and the inserts for three transformants with the correct restriction digestion patterns were confirmed by DNA sequencing. One clone with the desired sequence was selected and designated pWTY1470-09-05.

A 608 bp Bgl II fragment harboring the tri5 gene 5′ repeat was liberated from pWTY1470-09-05 by digestion with Bgl II, purified by 1.0% agarose gel electrophoresis using TBE buffer, excised from the gel, and agarose extracted using a MINELUTE® Gel Extraction Kit.

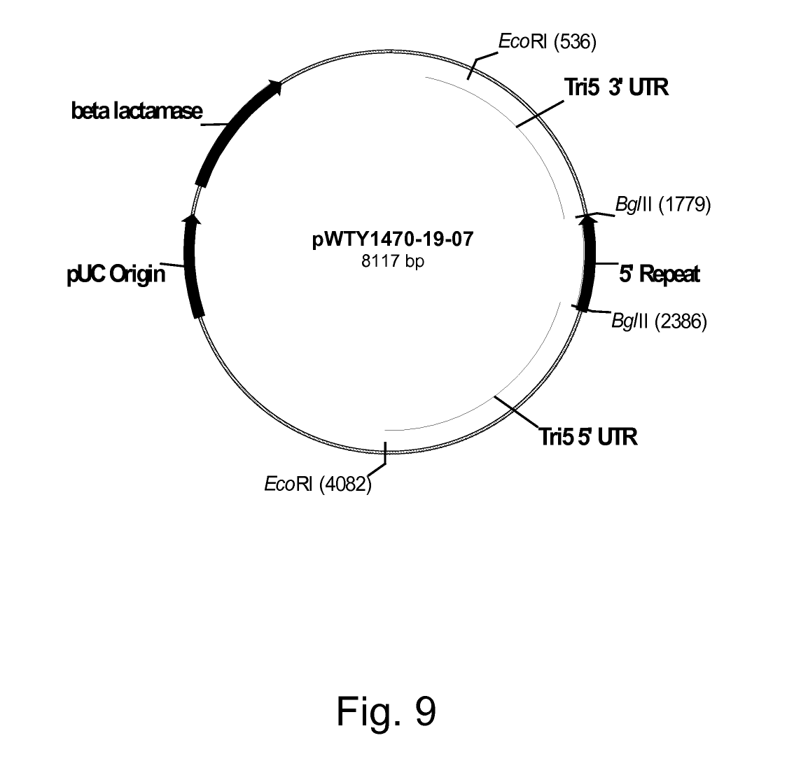

Plasmid pJRoy40 was linearized by digestion with Bgl II, after which it was dephosphorylated using shrimp alkaline phosphatase (Roche Diagnostics Corporation, Indianapolis, Ind., USA) according to the manufacturer's instructions, and purified using a QIAQUICK® PCR Purification Kit (QIAGEN Inc., Valencia, Calif., USA). Linearized pJRoy40 and the gel-purified Bgl II fragment were ligated together using T4 DNA ligase (New England Biolabs Inc., Ipswich, Mass., USA) according to the manufacturer's instructions. Transformation of E. coli SURE® chemically competent cells (Stratagene, LA Jolla, Calif., USA) was performed according to the manufacturer's directions. One transformant was confirmed by DNA sequencing to contain the desired vector, i.e., harboring the tri5 5′ and 3′ flanking sequences and, in addition, a repeat of a portion of the 5′ flanking sequence. The resulting plasmid was designated pWTY1470-19-07 (FIG. 9).

### Example 18

### Construction of Plasmid pWTY1515-02-01

Plasmid pWTY1470-19-07 was subjected to in vitro mutagenesis using a QUIKCHANGE® Site-Directed Mutagenesis Kit according to the manufacturer's instructions and forward and reverse primers shown below.

Forward primer:
(SEQ ID NO: 33)
5′-CAAGTAACAGACGCGACAGCTTGCAAAATCTTCGTTATCTGTG-3′
Reverse primer:
(SEQ ID NO: 34)
5′-CACAGATAACGAAGATTTTGCAAGCTGTCGCGTCTGTTACTTG-3′

The mutagenesis removed the Bgl II site at 1779 bp and rendered the Bgl II site at 2386 bp unique and usable in subsequent manipulations to insert fragments harboring thymidine kinase (tk) and hygromycin phosphotransferase (hpt) gene cassettes. The mutagenesis reaction was used to transform the kit-supplied E. coli XL10-GOLD® Ultra-competent cells (Stratagene, La Jolla, Calif., USA) according to the manufacturer's suggested protocol.

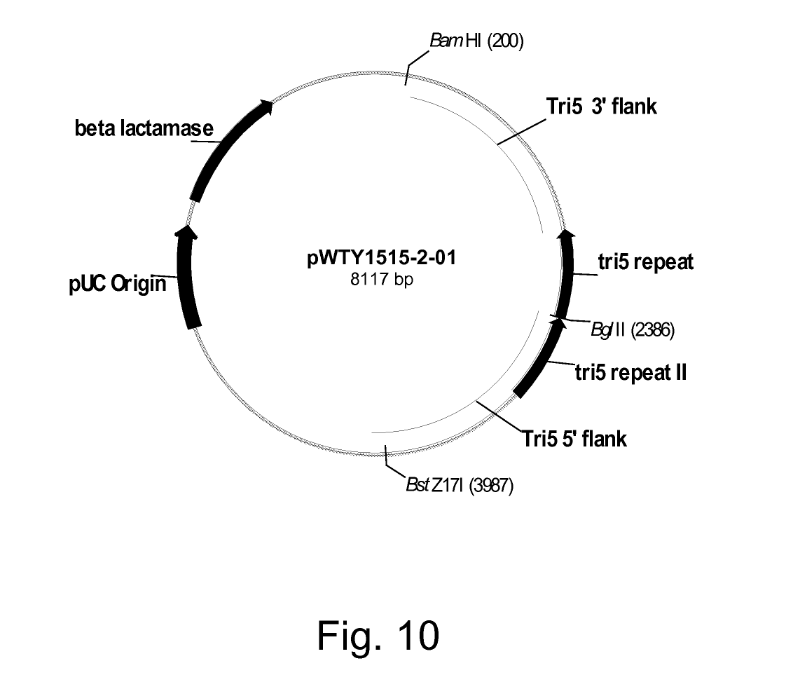

One transformant harboring the mutations indicated above, as verified by sequence analysis, was designated pWTY1515-02-01 (FIG. 10) and used as the backbone in Example 19.

### Example 19

### Generation of the Tris Deletion Vector pJfyS1579-21-16

An E. coli hygromycin phoshotransferase (hpt) gene cassette was PCR amplified from plasmid pEmY23 using an ADVANTAGE® GC Genomic PCR Kit (Clonetech, Palo Alto, Calif., USA) and gene-specific forward and reverse primers shown below. The underlined portion in the reverse primer is a Bgl II site for cloning.

Forward primer:
(SEQ ID NO: 35)
5′-TTGAACTCTCAGATCCCTTCATTTAAACGGCTTCACGGGC-3′
Reverse primer:
(SEQ ID NO: 36)
5′-CAGATAACGAAGATCTACGCCCTTGGGGTACCCAATATTC-3′

The PCR reaction contained 362 ng of pEmY23 as DNA template, 200 μm dNTP's, 1.1 mM magnesium acetate, 0.4 μM primers, 1× GC Reaction Buffer (Clonetech, Palo Alto, Calif., USA), 0.5 M GC Melt (Clonetech, Palo Alto, Calif., USA), and 1× GC Genomic Polymerase Mix (Clonetech, Palo Alto, Calif., USA) in a final volume of 50 μl.

The amplification reaction was incubated in an EPPENDORF® MASTERCYCLER® (Eppendorf, Munich, Germany) programmed for 1 cycle at 95° C. for 2 minutes; 25 cycles each at 94° C. for 30 seconds and 66° C. for 3 minutes; and 1 cycle at 66° C. for 3 minutes; and a hold at 4° C.

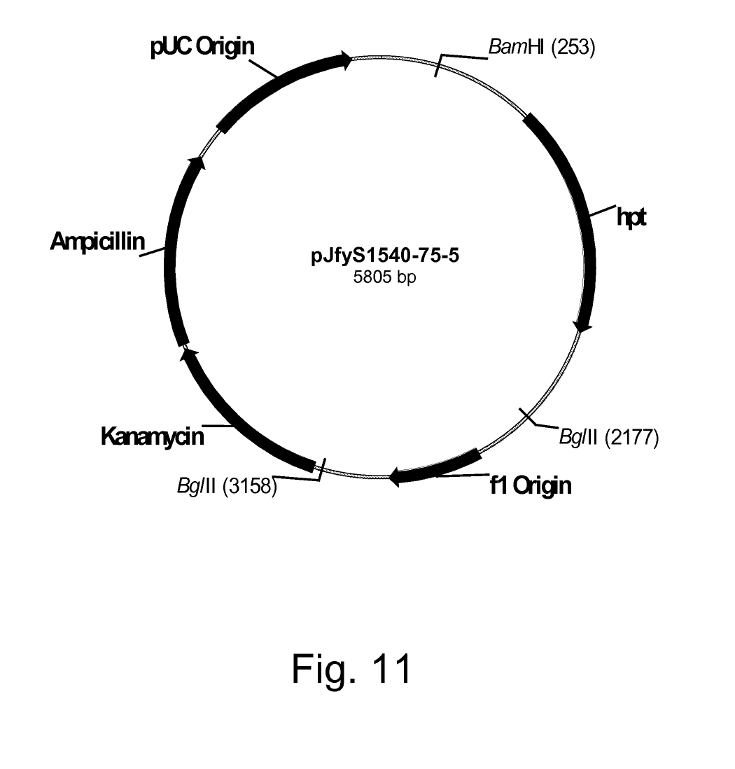

PCR products were separated by 1% agarose gel electrophoresis using TAE buffer. A fragment of approximately 1.9 kb was excised from the gel and agarose extracted using a MINIELUTE® Gel Extraction Kit (QIAGEN Inc., Valencia, Calif., USA). The fragment was cloned into pCR®2.1 using a TOPO® TA Cloning Kit according to the manufacturer's instructions. ONE SHOT® TOP10 competent cells (Invitrogen, Carlsbad, Calif., USA) were transformed with 2 μl of the TOPO® TA reaction. Sequence analysis of plasmid DNA from 8 transformants confirmed that there were no deviations from the expected sequence and the plasmid was designated pJfyS1540-75-5 (FIG. 11).

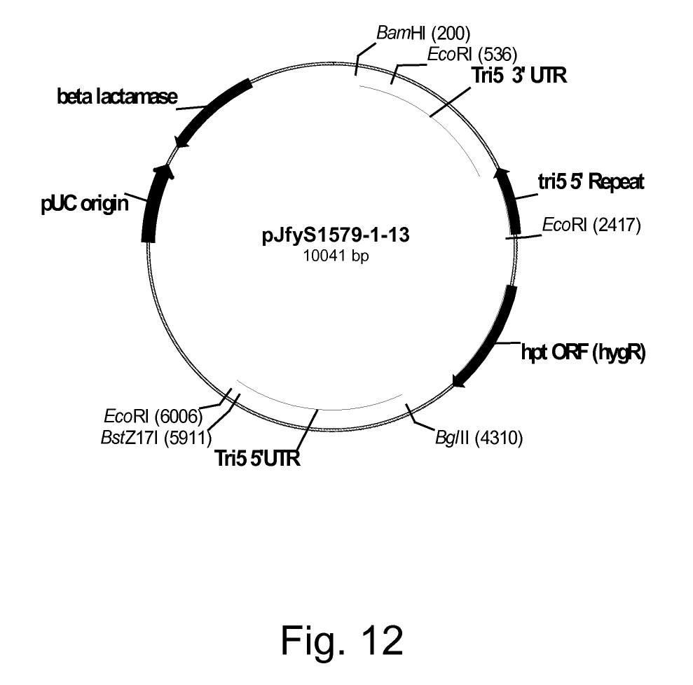

The hpt insert was liberated from pJfyS1540-75-05 by digestion with Bam HI and Bgl II and purified by 1% agarose gel electrophoresis in TAE buffer. A fragment of 1.9 kb was excised and agarose-extracted using a MINIELUTE® Gel Extraction Kit. A Rapid DNA Ligation Kit was used to ligate the fragment to Bgl II-linearized empty tri5 deletion vector pWTY1515-02-01 (Example 18), which had been dephosphorylated using calf intestine phosphatase. E. coli SURE® chemically competent cells were transformed with the ligation reaction and plasmid DNA from 24 of the resulting transformants was analyzed by restriction digestion with Eco RI to confirm the orientation of the insert. One of the transformants harboring the insert in the desired orientation was selected and designated pJfyS1579-1-13 (FIG. 12).

A Herpes simplex virus thymidine kinase (tk) gene (SEQ ID NO: 37 for the DNA sequence and SEQ ID NO: 38 for the deduced amino acid sequence) was PCR amplified using pWTY1449-2-1 as template and gene specific forward and reverse primers shown below. The bold sequence represents the introduced Bgl II site.

Forward primer:
(SEQ ID NO: 39)
5′-GCCGACTACTAGATCGACCGGTGACTCTTTCTGGCATGCG-3′
Reverse primer:
(SEQ ID NO: 40)
5′-CAGATAACGAAGATCTGAGAGTTCAAGGAAGAAACAGTGC-3′

The PCR reaction contained 1× HERCULASE® reaction buffer (Stratagene, La Jolla, Calif., USA), 200 μM dNTPs, 55 ng of pWTY1449-2-1, 0.2 μM primers, 2% DMSO, and 2.5 units of HERCULASE® DNA polymerase (Stratagene, La Jolla, Calif., USA) in a final volume of 50 μl.

The amplification reaction was incubated in an EPPENDORF® MASTERCYCLER® programmed for 1 cycle at 95° C. for 1 minute; 25 cycles each at 94° C. for 30 seconds, 60° C. for 30 seconds, and 68° C. for 2 minutes and 45 seconds; and 1 cycle at 68° C. for 2 minutes and 45 seconds; and a hold at 4° C.

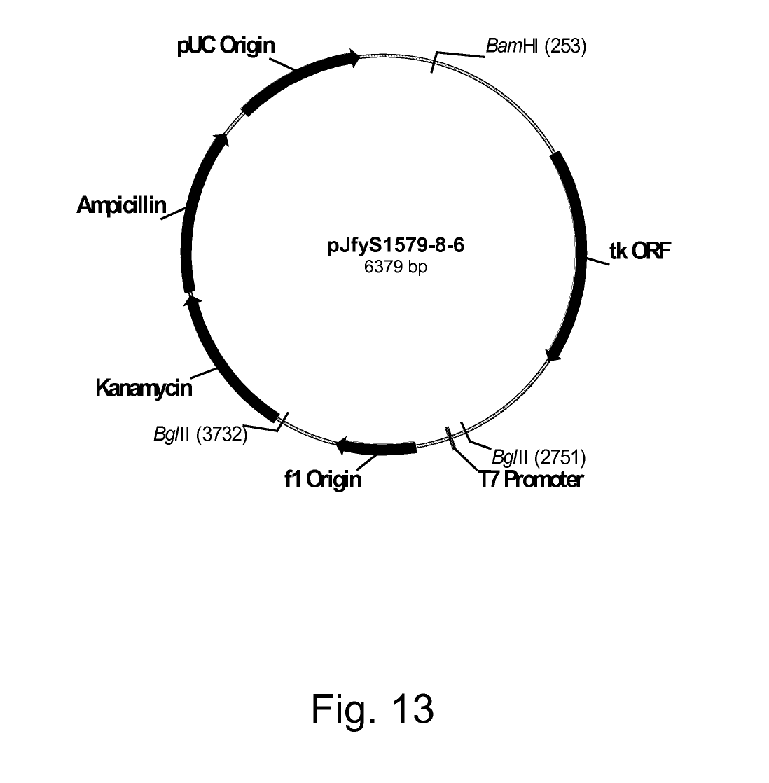

PCR products were separated by 1% agarose gel electrophoresis using TAE buffer. A fragment of approximately 2.8 kb was excised from the gel and purified using a MINIELUTE® Gel Extraction Kit. The fragment was cloned into pCR®2.1 using a TOPO® TA Cloning Kit. ONE SHOT® TOP10 competent cells (Invitrogen, Carlsbad, Calif., USA) were transformed with 2 μl of the TOPO® TA reaction. Sequence analysis of plasmid DNA from one of the transformants identified a mutation in the tk coding sequence (C1621G) resulting in an amino acid change of glycine to alanine. This mutation was corrected using a QUIKCHANGE® II XL Site-Directed Mutagenesis Kit (Stratagene, La Jolla, Calif., USA) according to the manufacturer's instructions and forward and reverse primers shown below. The lower case letter indicates the desired change. Sequence analysis of 16 clones resulted in the selection of one which was designated pJfyS1579-8-6 (FIG. 13).

Forward primer:
(SEQ ID NO: 41)
5′-CCCTGTTTCGGGgCCCCGAGTTGCTGG-3′
Reverse primer:
(SEQ ID NO: 42)
5′-CCAGCAACTCGGGGcCCCGAAACAGGG-3′

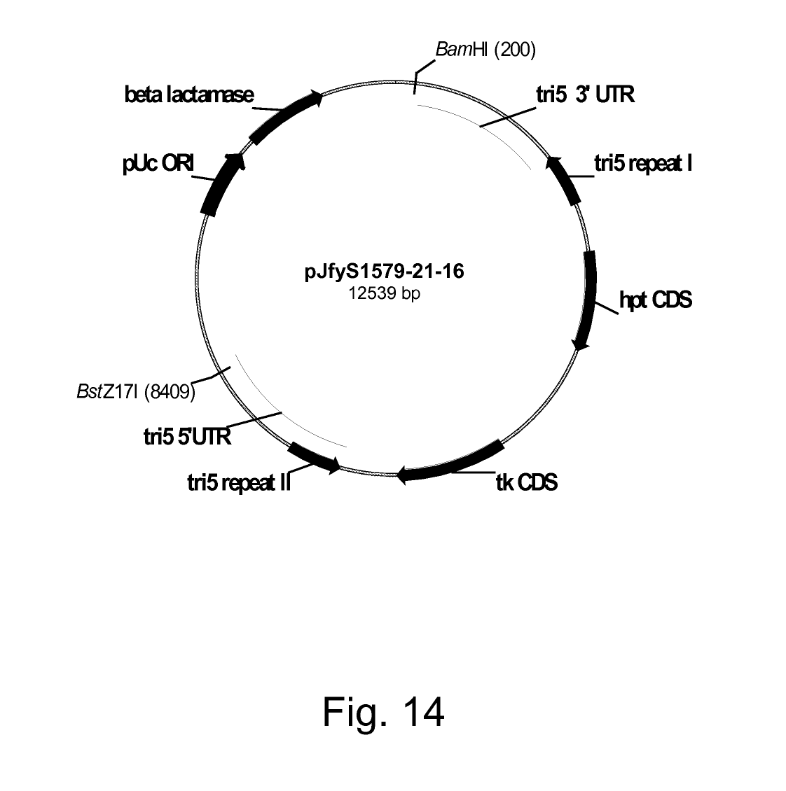

Plasmid pJfyS1579-08-06 was digested with Bam HI and Bgl II to liberate the 2.8 kb tk fragment and the fragment was purified as described above. This fragment was ligated to pJfyS1579-1-13, which had been linearized with Bgl II and treated with calf intestine phosphatase, using a QUICK LIGATION™ Kit and used to transform E. coli SURE® chemically competent cells according to the manufacturer's protocol. The resulting plasmid was designated pJfyS1579-21-16 (FIG. 14) and used as the tri5 deletion cassette.

### Example 20

### Fusarium venenatum Transformation Procedure

One hundred micrograms of each of the deletion cassettes described in the following examples were digested with either Bst Z171/Bam HI (Example 21) or Not I (Examples 24, 26, 37 and 39). Each digestion reaction was purified by 1% agarose gel electrophoresis in TAE buffer and a DNA band was extracted using a QIAQUICK® Gel Extraction Kit. The resulting purified DNA was concentrated in a 1.5 ml microfuge tube by ethanol precipitation with the addition of 10% reaction volume of 3 M sodium acetate pH 5 followed by 2.5 volumes of ice cold ethanol (94%) and incubation on ice for 20 minutes. The tube was then centrifuged at 15,000×g for 10 minutes in an EPPENDORF® 5424 bench-top centrifuge (Eppendorf, Hamburg, Germany). The supernatant was discarded and the pellet washed with 1 ml of ice cold 70% ethanol and centrifuged at 15,000×g for 5 minutes. The supernatant was discarded and the pellet allowed to air dry. The pellet was then resuspended in 70 μl of 10 mM Tris pH 8 buffer. The concentration of the resulting DNA containing solution was determined using a NANODROP® 1000 spectrophotometer (ThermoFischer Scientific, Waltham, Mass., USA).

Protoplasts of the appropriate recipient strain were generated by the following method. Spores were first obtained by inoculating 500 ml of RA medium (Example 21) or RA medium supplemented with 10 mM uridine (Examples 24, 26, 37 and 39) in a 2.8 L Fernbach flask with 15×1 cm² agar plugs of a 7-day old culture containing VNO₃RLMT medium and incubating the flask for 36 hours at 28° C. with shaking at 150 rpm. The spore culture was filtered through sterile MIRACLOTH™ and the spores captured on a MILLIPORE® STERICUP® 0.2 μm filter unit (Millipore, Bellerica, Mass., USA). The spores were washed with 200 ml of sterile glass distilled water and resuspended in 10 ml of sterile glass distilled water.

One ml of the spore solution was used to inoculate 100 ml of YP medium supplemented with 5% glucose (Example 21) or YP medium supplemented with 5% glucose and 10 mM uridine (Examples 24, 26, 37 and 39). The inoculated medium was incubated for 16 hours at 17° C. with shaking at 150 rpm. Cultures were filtered through MIRACLOTH™ to collect mycelia, which were transferred to a 50 ml polypropylene tube using a sterile spatula. The mycelia were resuspended in 20 ml of protoplasting solution, which contained 5 mg of NOVOZYME™ 234 per ml and 5 mg of GLUCANEX™ (both from Novozymes NS, Bagsvaerd, Denmark) in 1 M MgSO₄ per ml and transferred to 50 ml polypropylene tubes. The tubes were incubated at 29.5° C. with shaking at 90 rpm for one hour after which 30 ml of 1 M sorbitol were added. Then the tubes were centrifuged at 800×g for 10 minutes in a Sorvall RT 6000B swinging-bucket centrifuge (ThermoFischer Scientific, Waltham, Mass., USA). The supernatants were discarded and the protoplast pellets were washed twice with 30 ml of 1 M sorbitol. The tubes were centrifuged at 800×g for 5 minutes and the supernatants discarded. The protoplasts were resuspended in a solution of filter-sterilized 9:1:0.1 (v/v) STC:SPTC:DMSO at a concentration of 5×10⁷ per ml and frozen overnight at −80° C. at controlled rate freezing using a NALGENE™ Cryo 1° C. Freezing Container (ThermoFischer Scientific, Waltham, Mass., USA).

Transformation was accomplished by thawing the protoplasts on ice and adding 200 μl of the protoplasts to each of four 14 ml tubes. Five μg of DNA (in less than 10 μl) were added to the first three and no DNA was added to the fourth. Then 750 μl of SPTC were added to each tube and the tubes were inverted gently 6 times. The tubes were incubated at room temperature for 30 minutes and 6 ml of STC were added to each tube. Each transformation was divided into three parts and added to 150 mm diameter plates containing VNO₃RLMT medium supplemented with 125 μg of hygromycin per ml (Example 21) or VNO₃RLMT medium supplemented with 125 μg of hygromycin per ml and 10 mM uridine (Examples 24, 26, 37 and 39) and incubated at room temperature for 7 days.

### Example 21

### Construction of the Δtri5 Fusarium venenatum Strain JfyS1604-47-02

Fusarium venenatum A3/5 protoplasts were transformed with Bst Z171/Bam HI-linearized pJfyS1579-21-16 using the method described in Example 20. Transformants were selected on VNO₃RLMT plates containing 125 μg of hygromycin B per ml. After day 7, 48 out of 123 transformants were sub-cultured to a new plate containing the same medium. Eight transformants were then analyzed by Southern analysis as follows. Fungal biomass of these strains was generated by inoculating 25 ml of M400 medium with four 1 cm agar plugs from 7 day old transformants obtained as described above. The cultures were incubated for 3 days at 28° C. with shaking at 150 rpm. Agar plugs were removed and the cultures were filtered through MIRACLOTH™. Harvested biomass was frozen with liquid nitrogen and the mycelia were ground using a mortar and pestle.

Genomic DNA was isolated using a DNEASY® Plant Maxi Kit according to the manufacturer's instructions, except the lytic incubation period at 65° C. was extended to 1.5 hours from 10 minutes.

Two μg of genomic DNA were digested with 16 units of Sph 1 and 22 units of Dra I in a 50 μl reaction volume at 37° C. for 22 hours. The digestion was subjected to 1.0% agarose gel electrophoresis in TAE buffer. The DNA was fragmented in the gel by treating with 0.25 M HCl, denatured with 1.5 M NaCl-0.5 M NaOH, neutralized with 1.5 M NaCl-1 M Tris pH 8, and then transferred in 20×SSC to a NYTRAN® Supercharge nylon membrane using a TURBOBLOTTER™ Kit (both from Whatman, Kent, UK). The DNA was UV cross-linked to the membrane using a UV STRATALINKER™ and pre-hybridized for 1 hour at 42° C. in 20 ml of DIG Easy Hyb.

A PCR probe for the 3′ flanking sequence of the tri5 gene was generated using the following forward and reverse primers.

Forward primer:
(SEQ ID NO: 43)
5′-GTGGGAGGATCTGATGGATCACCATGGGC-3″
Reverse primer:
(SEQ ID NO: 44)
5′-CCGGGTTTCGTTCCGAACGATCTTTACAAGG-3′

The probe was generated using a PCR Dig Probe Synthesis Kit according to the manufacturer's instructions. The probe was purified by 1.2% agarose gel electrophoresis in TAE buffer and the band corresponding to the probe was excised and agarose-extracted using a MINELUTE® Gel Extraction Kit. The probe was boiled for 5 minutes and added to 10 ml of DIG Easy Hyb to produce the hybridization solution. Hybridization was performed at 42° C. for 15-17 hours. The membrane was then washed under high stringency conditions in 2×SSC plus 0.1% SDS for 5 minutes at room temperature followed by two washes in 0.1×SSC plus 0.1% SDS for 15 minutes each at 65° C. The probe-target hybrids were detected by chemiluminescent assay (Roche Diagnostics, Indianapolis, Ind., USA) according to the manufacturer's instructions.

One transformant, Fusarium venenatum JfyS1579-43-23, harboring the deletion cassette in a single copy in the tri5 locus, as determined by Southern analysis, was sporulated by cutting four 1 cm² plugs using sterile toothpicks from a 7 day-old plate containing VNO₃RLMT medium and transferring them to a 125 ml baffled shake flask containing 25 ml of RA medium. The flask was incubated at 28° C. with shaking at 150 rpm for 48 hours. The spore culture was filtered through sterile MIRACLOTH™ and collected in a 50 ml polypropylene tube. The concentration of spores was determined using a hemocytometer and 10⁵ spores (in one ml) were transferred to a 150 mm plate containing VNO₃RLMT medium supplemented with 50 μM FdU and incubated for 4 days at 28° C. Spore isolates were picked using sterile toothpicks and transferred to a new plate containing VNO₃RLMT medium supplemented with 10 μM FdU and allowed to grow for 7 days at 24-28° C.

Genomic DNA was extracted from 7 spore isolates and Southern analyses performed as described above to insure the cassette's correct excision from the genome. All spore isolates analyzed by Southern blots had excised the cassette leaving behind one repeat as expected. One spore isolate was spore purified once by inducing sporulation in the strain as described in the preceding paragraph, and the spore concentration was determined using a hemocytometer and diluted to 40 spores per ml. One ml of the diluted spore solution was plated to 150 mm plates containing VNO₃RLMT medium and the plates were incubated at 28° C. for 4 days. Spore isolates were sub-cultured to new plates containing VNO₃RLMT medium and one spore isolate, designated Fusarium venenatum JfyS1604-17-02 (Δtri5), was used as the starting strain for deletion of the pyrG gene.

### Example 22

### Construction of a Universal Deletion Vector Harboring the Thymidine Kinase (tk) Negative Selection Marker and Hygromycin Phosphotransferase (hpt) Positive Selection Marker

A universal deletion vector harboring both the thymidine kinase (tk) and hygromycin phosphotransferase (hpt) markers was constructed to facilitate assembly of subsequent deletion plasmids. Flanking sequences for 5′ and 3′ regions of the gene targeted for deletion can be easily ligated to the vector following digestion of the latter with Pme I or Asc I (for 5′ flanking sequences) and Sbf I or Swa I (for 3′ flanking sequences).

In order to PCR-amplify the direct repeats derived from the 5′ flanking region of the Fusarium venenatum pyrG gene, 50 picomoles of the primers shown below were used in two PCR reactions containing 50 ng of pDM156.2, 1× Pfx Amplification Buffer (Invitrogen, Carlsbad, Calif., USA), 6 μl of a 10 mM blend of dNTPs, 2.5 units of PLATINUM® Pfx DNA polymerase (Invitrogen, Carlsbad, Calif., USA), and 1 μl of 50 mM MgSO₄ in a total volume of 50 μl.

### Primers:

Repeat #1
Sense Primer:
(SEQ ID NO: 45)
5′-GTTTAAACGGCGCGCC CGACAAAACAAGGCTACTGCAGGCAGG-3′
Antisense Primer:
(SEQ ID NO: 46)
5′-TTGTCGCCCGGG AATACTCCAACTAGGCCTTG-3′
Repeat #2
Sense Primer:
(SEQ ID NO: 47)
5′-AGTATTCCCGGG CGACAAAACAAGGCTACTGCA-3′
Antisense Primer:
(SEQ ID NO: 48)
5′-ATTTAAATCCTGCAGG AATACTCCAACTAGGCCTTG-3′

The amplification reactions were incubated in an EPPENDORF® MASTERCYCLER® programmed as follows. For repeat #1:1 cycle at 98° C. for 2 minutes; and 5 cycles each at 94° C. for 30 seconds, 55° C. for 30 seconds, and 68° C. for 1 minute. This was followed by 35 cycles each at 94° C. for 30 seconds, 59° C. for 30 seconds, and 68° C. for 1 minute. For repeat #2 the cycling parameters were: 1 cycle at 98° C. for 2 minutes; and 5 cycles each at 94° C. for 30 seconds, 55° C. for 30 seconds, and 68° C. for 1 minute. This was followed by 35 cycles each at 94° C. for 30 seconds, 56° C. for 30 seconds, and 68° C. for 1 minute. After the 35 cycles both reactions (i.e., repeats # 1 and #2) were incubated at 68° C. for 10 minutes and then cooled at 10° C. until being further processed.

PCR products from both reactions were separated by 0.8% GTG-agarose (Cambrex Bioproducts, East Rutherford, N.J., USA) gel electrophoresis using TAE buffer. For repeat #1 and repeat #2, fragments of approximately 0.26 kb were excised from the gel and purified using Ultrafree®-DA spin cups (Millipore, Billerica, Mass., USA) according to the manufacturer's instructions. Ten microliters of each purified repeat were then used in a single overlapping PCR reaction containing 1× Pfx Amplification Buffer, 6 μl of a 10 mM blend of dATP, dTTP, dGTP, and dCTP, 2.5 units of PLATINUM® Pfx DNA polymerase, and 1 μl of 50 mM MgSO₄ in a total volume of 50 μl.

The amplification reaction was incubated in an EPPENDORF® MASTERCYCLER® programmed for 1 cycle at 98° C. for 2 minutes; and 5 cycles each at 94° C. for 30 seconds, 50° C. for 30 seconds, and 68° C. for 1 minute. The reaction was then mixed with a pre-warmed solution containing 50 picomoles of the sense primer for repeat #1 and 50 picomoles of the anti-sense primer for repeat #2, 1× Pfx Amplification Buffer, 6 μl of a 10 mM dNTPs, 2.5 units of PLATINUM® Pfx DNA polymerase, and 1 μl of 50 mM MgSO₄ in a final volume of 50 μl.

The new 100 μl amplification reaction was incubated in an EPPENDORF® MASTERCYCLER® programmed for 35 cycles each at 94° C. for 30 seconds, 58° C. for 30 seconds, and 68° C. for 1 minute. After 35 cycles, the reaction was incubated at 68° C. for 10 minutes and then cooled at 10° C. until being further processed. A 0.5 kb PCR product (harboring the repeat assembly) was isolated by 0.8% GTG-agarose gel electrophoresis as described above.

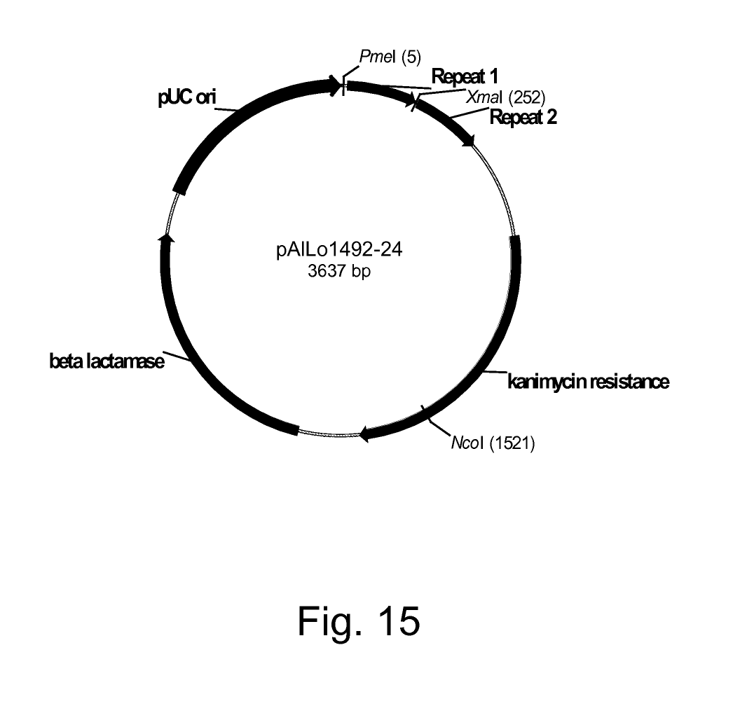

Plasmid pCR4 (Invitrogen, Carlsbad, Calif., USA) was used as the source of the vector backbone for the construction of the universal deletion vector. To remove the non-essential portions of the pCR4 DNA, 2.5 μg of plasmid pTter61C (WO 2005/074647) were digested sequentially with Bsp LU11I and Bst XI. The digested vector was then treated with Antarctic phosphatase (New England Biolabs Inc., Ipswich, Mass., USA). The 3.1 kb digested backbone was isolated by 0.8% GTG-agarose gel electrophoresis as described above. The purified repeat assembly was then ligated to the purified vector backbone with a Rapid Ligation Kit (Roche Diagnostics Corporation, Indianapolis, Ind., USA). The ligation reaction consisted of 75 ng of purified vector backbone and 3 μl of the purified repeat assembly. One microliter of this ligation reaction was used to transform chemically competent SOLOPACK® Supercompetent cells (Stratagene, Carlsbad, Calif., USA) using the manufacturer's suggested protocols. Twenty four transformants were analyzed by Nco I/Pme I restriction digestion. Twenty three out of twenty four transformants had the expected restriction digestion pattern. Clone pFvRs #10 was selected at random for sequencing to confirm that there were no PCR-induced errors. Sequencing analysis showed that the repeat assembly in clone pFvRs #10 had the expected sequence, and this was therefore selected as the backbone of the Fusarium venenatum universal vector and designated pAlLo1492-24 (FIG. 15).

The cassette harboring the hygromycin phosphotransferase (hpt) gene was PCR amplified from pEmY23 using the gene-specific forward and reverse primers shown below. The underlined sequence represents a Xma I site and the bold letters represent a Bgl II site. The four “a”s at each 5′ end allow for subsequent digestion of the terminal ends of the PCR product.

Forward primer:
(SEQ ID NO: 49)
5′-aaaacccgggCCTTCATTTAAACGGCTTCACGGGC-3′
Reverse primer:
(SEQ ID NO: 50)
5′-aaaacccgggAGATCTACGCCCTTGGGGTACCCAATATTC-3′

The amplification reaction contained 60 ng of pEmY23, 200 μm dNTPs, 1 mM magnesium acetate, 0.4 μM primers, 1× Pfx Amplification Buffer, 0.5 M GC Melt, and 2.5 units of PLATINUM® Pfx polymerase in a final volume of 50 μl. The reaction was incubated in an EPPENDORF® MASTERCYCLER® programmed for 1 cycle at 95° C. for 2 minutes; 10 cycles each at 94° C. for 30 seconds, 60° C. for 30 seconds, and 68° C. for 1 minute 50 seconds; and 1 cycle at 68° C. for 7 minutes followed by holding at 4° C.

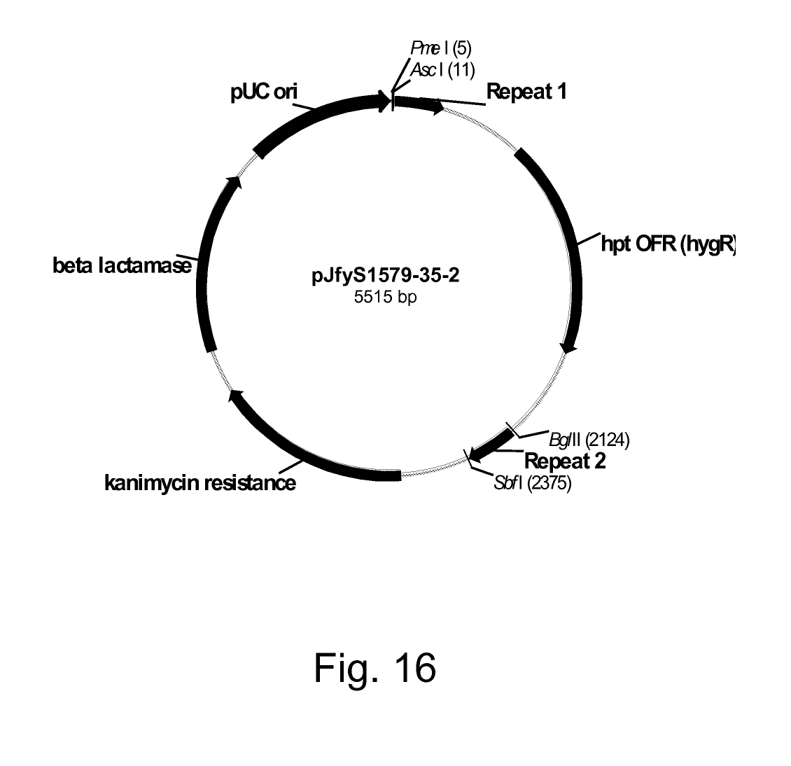

PCR products were separated by 1% agarose gel electrophoresis using TAE buffer. A fragment of approximately 1.8 kb was excised from the gel and agarose-extracted using a MINIELUTE® Gel Extraction Kit. The gel-purified PCR product was subsequently digested with Xma I and run on a 1% agarose gel and gel-purified again as above. A QUICK LIGATION™ Kit was used to ligate the hpt PCR product to Xma I-linearized pAlLo1492-24, which had been treated with calf intestine phosphatase. The resulting plasmid was designated pJfyS1579-35-2 (FIG. 16) and was used as the recipient for the insertion of the thymidine kinase gene.

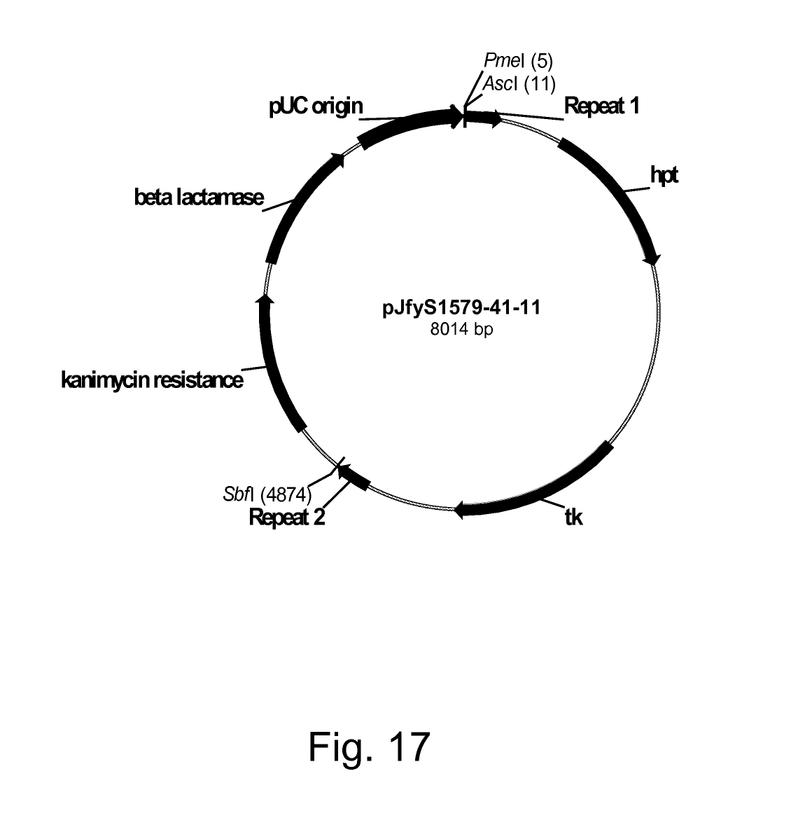

The source of the Herpes simplex virus tk cassette was plasmid pJfyS1579-08-06 (Example 19), from which the insert was liberated by digestion with Bam HI and Bgl II. The digestion products were separated by 1% agarose gel electrophoresis using TAE buffer, and a fragment corresponding to the 2.8 kb tk gene insert was excised and agarose-extracted using a MINELUTE® Gel Extraction Kit. A QUICK LIGATION™ Kit was used to ligate the tk gene cassette to Bgl II-linearized pJfyS1579-35-02, which had been treated with calf intestine phosphatase. The resulting plasmid was designated pJfyS1579-41-11 (FIG. 17) and this was used as the starting point for construction of the pyrG, amyA, alpA, and dps1 deletion vectors.

### Example 23

### Generation of the pyrG Deletion Vector pJfyS1604-55-13

The 3′ flanking sequence of the Fusarium venenatum A3/5 pyrG gene (SEQ ID NO: 51 for the DNA sequence and SEQ ID NO: 52 for the deduced amino acid sequence) was amplified using an EXPAND® High Fidelity PCR System (Roche Diagnostics Corporation, Indianapolis, Ind., USA) and gene-specific forward and reverse primers shown below. The underlined portion is a Sbf I site introduced for cloning and the italicized portion is a Not I site introduced for later digestion to remove the pCR®2.1 portion of the plasmid before transformation.

Forward primer:
(SEQ ID NO: 53)
5′-aaaaaacctgcaggATCCTGCGCGGACTCTTGATTATTT-3′
Reverse primer:
(SEQ ID NO: 54)
5′-aaaaaacctgcagggcggccgcAATTCCATTCCTGTAGCTGAGTAT
A-3′

The amplification reaction contained 125 ng of Fusarium venenatum A3/5 genomic DNA, 200 μm dNTP's, 0.4 μM primers, 1× EXPAND® Buffer (Roche Diagnostics Corporation, Indianapolis, Ind., USA) with 5 mM MgCl₂, and 2.5 units of EXPAND® DNA polymerase (Roche Diagnostics Corporation, Indianapolis, Ind., USA) in a final volume of 50 μl. The amplification reaction was incubated in an EPPENDORF® MASTERCYCLER® programmed for 1 cycle at 95° C. for 2 minutes; 10 cycles each at 94° C. for 30 seconds, 54° C. for 30 seconds, and 72° C. for 1 minute; and 20 cycles each at 94° C. for 30 seconds, 54° C. for 30 seconds, and 72° C. for 1 minute and 10 seconds.

PCR products were separated by 1% agarose gel electrophoresis using TAE buffer and a 0.7 kb fragment was excised and agarose extracted using a MINELUTE® Gel Extraction Kit.

The 0.7 kb PCR product was digested with Sbf I and purified by 1% agarose gel electrophoresis using TAE buffer. A fragment of approximately 0.7 kb was excised from the gel and further purified using an Ultrafree®-DA spin cup. The 0.7 kb fragment was ligated to pJfyS1579-41-11 (which had been digested with Sbf I and dephosphorylated using calf intestine phosphatase) using a QUICK LIGATION™ Kit and the ligation mixture used to transform E. coli SURE® chemically competent cells according to the manufacturer's protocol. The resulting plasmid was designated pJfyS1604-35-13.

The 5′ pyrG flanking sequence was amplified from pEmY23 (Example 13) using an EXPAND® High Fidelity PCR System and gene-specific forward and reverse primers shown below. The underlined portion is a Pme I site introduced for cloning and the italicized portion is a Not I site introduced for later digestion to remove the beta-lactamase gene prior to fungal transformation.

Forward primer:
(SEQ ID NO: 55)
5′-aaaaaagtttaaacgcggccgcCTGTTGCCTTTGGGCCAATCAATG-
3′
Reverse primer:
(SEQ ID NO: 56)
5′-aaaaaagtttaaacCTAGTTGGAGTATTGTTTGTTCTT-3′

The amplification reaction contained 20 ng of pEmY23, 200 μm dNTP's, 0.4 μM primers, 1× EXPAND® Buffer with 15 mM MgCl₂, and 2.5 units of EXPAND® DNA polymerase.

The amplification reaction was incubated in an EPPENDORF® MASTERCYCLER® programmed for 1 cycle at 95° C. for 2 minutes; 10 cycles each at 94° C. for 30 seconds, 53° C. for 30 seconds, and 72° C. for 40 seconds; and 20 cycles each at 94° C. for 30 seconds, 53° C. for 30 seconds, and 72° C. for 40 seconds plus an additional 10 seconds per subsequent cycle.

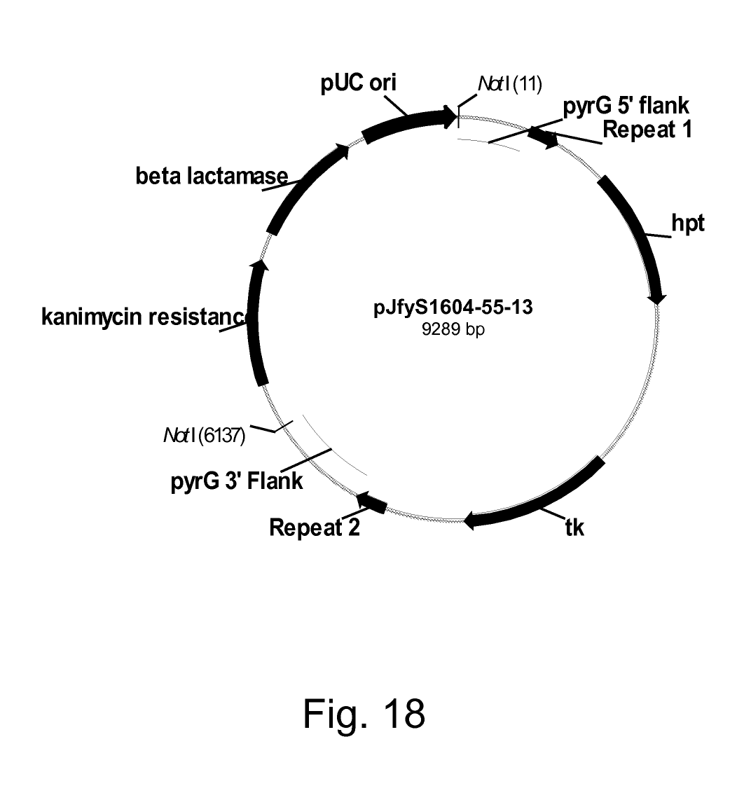

The PCR product was purified using a MINELUTE® PCR Purification Kit (QIAGEN Inc., Valencia, Calif., USA). The purified PCR products were digested with Pme I and separated by 1% agarose gel electrophoresis using TAE buffer. A fragment of approximately 0.5 kb was excised from the gel and agarose extracted using a MINELUTE® Gel Extraction Kit. The 0.5 kb fragment was ligated to Pme I digested and calf intestine phosphatase treated pJfyS1604-35-13 using a QUICK LIGATION™ Kit. The ligation reaction contained 50 ng of vector, 20 ng of insert, 1× QUICK LIGATION™ Reaction Buffer (New England Biolabs Inc., Ipswich, Mass., USA), and 10 units of Quick T4 DNA Ligase in a 20 μl reaction volume. The reaction was incubated at room temperature for 5 minutes and 2 μl of the ligation were used to transform E. coli SURE® chemically competent cells according to the manufacturer's Instructions. Sequence analysis was used to identify transformants containing the insert in the desired orientation and to confirm the absence of PCR errors. The resulting plasmid was designated pJfyS1604-55-13 (FIG. 18) and was used as the pyrG gene deletion cassette.

### Example 24

### Generation of Δtri5 ΔpyrG Fusarium venenatum Strain JfyS1643-18-2

Fifty-one putative transformants of Fusarium venenatum JfyS1604-17-2 (Δtri5), transformed with Not I-digested and gel-purified pJfyS1604-55-13 according to the procedure described in Example 20, were transferred from transformation plates with sterile toothpicks to new plates containing VNO₃RLMT medium supplemented with 125 μg of hygromycin B per ml and 10 mM uridine and grown at 24-28° C. for 7 days. Transformants were then analyzed phenotypically by transferring a plug to each of two VNO₃RLMT plates, one with and one without uridine (10 mM). Nine transformants displaying no or poor growth on the plates without uridine were then analyzed by Southern analysis. Genomic DNA from each of the 9 transformants was extracted as described in Example 21 and 2 μg of each were digested with 28 units of Mfe 1 and 14 units of Dra I. A PCR probe to the 3′ flanking sequence of the pyrG gene was generated according to the method described in Example 21 using the following forward and reverse primers:

| | |
|---|---|
| Forward primer: | |
| 5′-GGATCATCATGACAGCGTCCGCAAC-3′ | (SEQ ID NO: 57) |
| Reverse primer: | |
| 5′-GGCATAGAAATCTGCAGCGCTCTCT-3′ | (SEQ ID NO: 58) |

Southern analysis indicated that 2 of the 9 uridine auxotrophs harbored the deletion cassette in a single copy while the remainder had sustained ectopic integrations of the cassette. One transformant, Fusarium venenatum JfyS1604-85-5, was sporulated as described in Example 5 in RA medium with 10 mM uridine, and 10⁵ spores were plated to a 150 mm plate containing VNO₃RLMT medium supplemented with 50 μM FdU and 0.1 mM uridine. The spore isolates obtained were sub-cultured to a new plate containing VNO₃RLMT medium supplemented with 10 μM FdU and 0.1 mM uridine and analyzed subsequently by Southern analysis to insure correct excision from the genome.

The analyzed strains had all excised the cassette correctly and one strain, Fusarium venenatum JfyS1643-10-3, was sporulated as described in the preceding paragraph. The spore concentration was determined using a hemocytometer and the stock solution diluted to a concentration of 40 spores/ml. One ml was plated to 150 mm plates containing VNO₃RLMT medium supplemented with 10 mM uridine. Resulting spore colonies were sub-cultured to a new plate containing VNO₃RLMT medium supplemented with 10 mM uridine and one spore isolate, Fusarium venenatum JfyS1643-18-2 (Δtri5 ΔpyrG), was used as the strain for deletion of the Fusarium venenatum alpha-amylase A gene (amyA).

### Example 25

### Generation of the amyA Deletion Vector pJfyS1604-17-2

In order to obtain upstream and downstream flanking sequence information for complete removal of the Fusarium venenatum amyA gene (SEQ ID NO: 59 for the DNA sequence and SEQ ID NO: 60 for the deduced amino acid sequence), a GENOME WALKER™ Universal Kit (Clonetech, Palo Alto, Calif., USA) was used. Each Fusarium venenatum A3/5 genomic DNA library, generated with the kit, was subjected to two rounds of PCR for the 5′ flanking sequence using a 5′ gene-specific primer and a 5′ nested primer shown below. The 3′ flanking sequence was obtained using a 3′ gene-specific primer and a 3′ nested primer shown below.

5′ gene-specific primer:
(SEQ ID NO: 61)
5′-GAGGAATTGGATTTGGATGTGTGTGGAATA-3′
5′ nested primer:
(SEQ ID NO: 62)
5′-GGAGTCTTTGTTCCAATGTGCTCGTTGA-3′
3′ gene-specific primer:
(SEQ ID NO: 63)
5′-CTACACTAACGGTGAACCCGAGGTTCT-3′
3′ nested primer:
(SEQ ID NO: 64)
5′-GCGGCAAACTAATGGGTGGTCGAGTTT-3′

The primary PCR reactions contained 1× HERCULASE® Reaction Buffer, 2 μl of each genomic DNA library (generated as described in the kit), 200 nM kit-supplied AP1 (adaptor primer 1), 200 nM gene specific primer (above), 200 μM dNTPs, and 2.5 units of HERCULASE® DNA polymerase in a 50 μl reaction volume.

The primary amplifications were performed in an EPPENDORF® MASTERCYCLER® programmed for 7 cycles each at 94° C. for 25 seconds, 72° C. for 3 minutes, and 32 cycles each at 94° C. for 25 seconds and 67° C. for 3 minutes, and one cycle at 67° C. for 7 minutes.

The secondary PCR reaction contained 1× HERCULASE® Reaction Buffer, 1 μl of each primary PCR reaction (above), 200 nM kit-supplied AP2 (adaptor primer 2), 200 nM gene specific nested primer (above), 200 μM dNTPs, and 2.5 units of HERCULASE® DNA polymerase in a 50 μl reaction volume.

The secondary amplifications were performed in an EPPENDORF® MASTERCYCLER® programmed for 5 cycles each at 94° C. for 25 seconds, and 72° C. for 3 minutes, and 20 cycles each at 94° C. for 25 seconds and 67° C. for 3 minutes, and one cycle at 67° C. for 7 minutes.

PCR products were separated by 1% agarose gel electrophoresis using TAE buffer. A fragment of approximately 0.7 kb was excised from the gel and purified using a MINIELUTE® Gel Extraction Kit according to the manufacturer's instructions. The PCR product was sequenced directly using the corresponding nested primer described above and the kit-supplied primer 2. The obtained sequence was used to design primers to amplify a 1 kb region of the 5′ flanking sequence and a 0.7 kb region of the 3′ flanking sequence of the amyA gene for insertion into the empty deletion vector pJfyS1579-41-11.

The amyA 3′ flanking sequence was PCR amplified from Fusarium venenatum A3/5 genomic DNA using forward and reverse primers shown below.

Forward primer:
(SEQ ID NO: 65)
5′-AAAAAAcctgcaggTAATGGGTGGTCGAGTTTAAAAGTA-3′
Reverse primer:
(SEQ ID NO: 66)
5′-AAAAAAcctgcagggcggccgcTTTAAGCATCATTTTTGACTACGCA
C-3′

The underlined letters represent a Not I site for later beta-lactamase removal, and the italicized letters represent a Sbf I site for vector cloning.

The amplification reaction contained 1× HERCULASE® Reaction Buffer, 120 ng of genomic DNA template, 400 nm primers, 200 μM dNTPs, and 2.5 units of HERCULASE® DNA polymerase. The amplification reaction was incubated in an EPPENDORF® MASTERCYCLER® programmed for 1 cycle at 95° C. for 2 minutes; 10 cycles each at 94° C. for 30 seconds, 55° C. for 30 seconds, and 72° C. for 1 minute; and 20 cycles each at 94° C. for 30 seconds, 55° C. for 30 seconds, and 72° C. for 1 minute and 10 seconds.

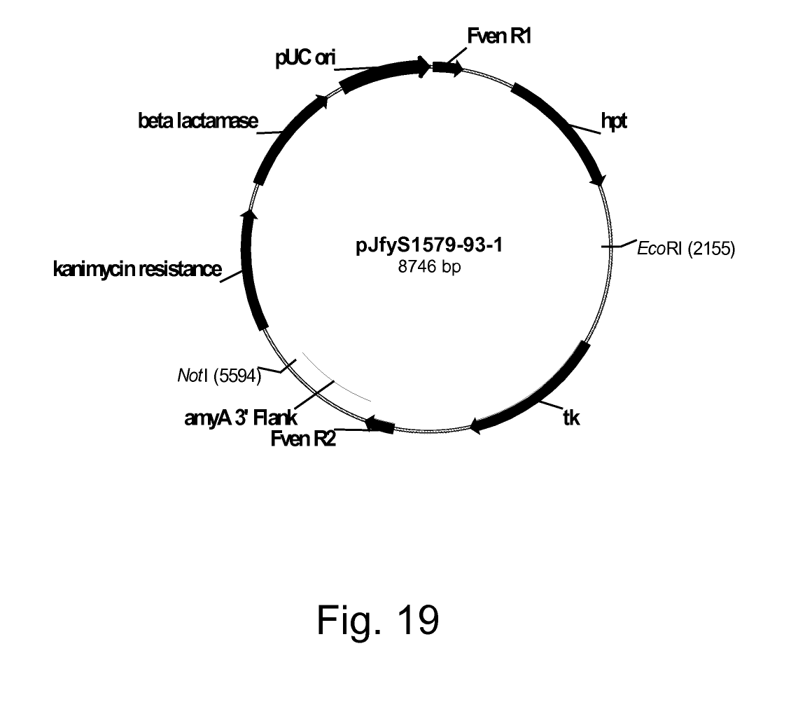

PCR products were separated by 1% agarose gel electrophoresis using TAE buffer. A fragment of approximately 0.7 kb was excised from the gel and agarose extracted using a MINIELUTE® Gel Extraction Kit. The PCR fragment was digested with Sbf I to produce sticky ends. This fragment was inserted into Sbf I-linearized, calf intestine phosphatase-treated universal deletion vector pJfyS1579-41-11. The ligation reaction contained 80 ng of vector, 80 ng of insert, 1× QUICK LIGATION™ Reaction Buffer, and 10 units of Quick T4 DNA Ligase in a 20 μl reaction volume. A 1.5 μl volume of the ligation reaction was used to transform 100 μl of E. coli SURE® chemically competent cells according to the manufacturer's instructions. Clones were screened for insert orientation using restriction analysis with Eco RI and sequence analysis, which identified a clone devoid of PCR errors. This plasmid was designated pJfyS1579-93-1 (FIG. 19) and used as the recipient for insertion of the 5′ amyA flanking sequence.

The 5′ amyA flanking sequence was PCR amplified using forward and reverse primers shown below. The underlined bases represent a Not I site for bla gene removal and the other lower case letters represent a Pme I site to insure the fragment was blunt for cloning into a blunt vector site.

Forward primer:
(SEQ ID NO: 67)
5′-AAAAAAgtttaaacGCGGCCGCTTGATTATGGGATGACCCCAGACAA
GTGGT-3′
Reverse primer:
(SEQ ID NO: 68)
5′-AAAAAAgtttaaacCCGCACGAGCGTGTTTCCTTTTCATCTCG-3′

The PCR amplification was similar to that described above except for different cycling parameters. The amplification reaction was incubated in an EPPENDORF® MASTERCYCLER® programmed for 1 cycle at 95° C. for 2 minutes; 10 cycles each at 94° C. for 30 seconds, 55° C. for 30 seconds, and 72° C. for 1 minute 15 seconds; and 20 cycles each at 94° C. for 30 seconds, 55° C. for 30 seconds, and 72° C. for 1 minute 15 seconds with an additional 10 seconds per subsequent cycle.

PCR products were separated by 1% agarose gel electrophoresis using TAE buffer. A fragment of approximately 1 kb was excised from the gel and agarose-extracted using a MINIELUTE® Gel Extraction Kit. The 1 kb fragment was digested with Pme Ito create blunt ends and the insert was cloned into Pme I-digested, calf intestine phosphatase-dephosphorylated pJfyS1579-93-1.

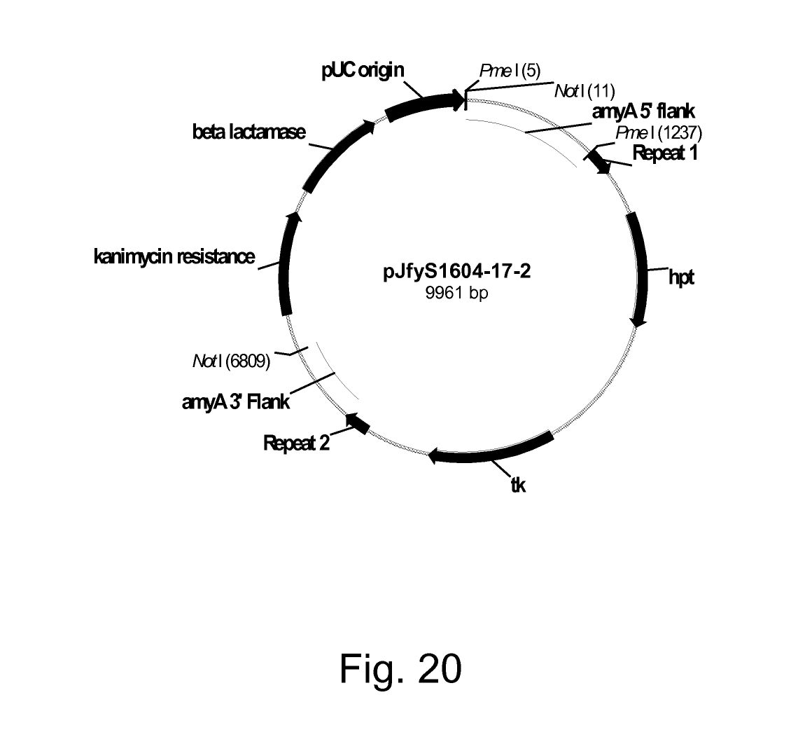

The ligation reaction contained 75 ng of vector, 100 ng of insert, 1× QUICK LIGATION™ Reaction Buffer, and 10 units of Quick T4 DNA Ligase in a 20 μl reaction volume. After a 5 minute incubation, 2 μl of the ligation reaction was used to transform 100 μl of E. coli SURE® chemically competent cells according to the manufacturer's instruction. Sequence analysis was used to confirm that the insert was in the correct orientation and the absence of PCR errors. The resulting vector identified was designated pJfyS1604-17-2 (FIG. 20).

### Example 26

### Generation of Δtri5 ΔpyrG ΔamyA Fusarium venenatum Strain JfyS1643-95-04

Five putative transformants of Fusarium venenatum JfyS1643-18-02 (Δtri5 ΔpyrG), transformed with Not I-digested and gel-purified pJfyS1604-17-02 according to the procedure described in Example 20, were transferred from transformation plates with sterile toothpicks to new plates containing VNO₃RLMT medium supplemented with 125 μg of hygromycin B per ml and 10 mM uridine and incubated at 24-28° C. for 7 days. For Southern analysis, 2 μg of genomic DNA were digested with 25 units of Ssp I. A DIG probe to the 5′ flanking sequence of the amyA gene was generated according to the method described in Example 21 using the forward and reverse primers shown below.

| | |
|---|---|
| Forward primer: | |
| 5′-GGATCATCATGACAGCGTCCGCAAC-3′ | (SEQ ID NO: 69) |
| Reverse primer: | |
| 5′-GGCATAGAAATCTGCAGCGCTCTCT-3′ | (SEQ ID NO: 70) |

Southern analysis was performed as described in Example 21 and the results indicated that two of the five transformants had a replaced coding sequence with a single integration of the deletion cassette. A primary transformant designated Fusarium venenatum JfyS1643-73-02 was sporulated as described in Example 5 and 10⁵ spores were plated to a 150 mm diameter plate containing VNO₃RLMT medium supplemented with 50 μM FdU and 0.1 mM uridine. Spore isolates obtained were sub-cultured to a new plate containing VNO₃RLMT medium supplemented with 10 μM FdU and 0.1 mM uridine.

Two Fusarium venenatum spore isolates (JfyS1643-83-02 and JfyS1643-83-04) were spore purified once resulting in strains JfyS1643-95-1 and JfyS1643-95-2 (from JfyS1643-83-02) and Jfys1643-95-04 (from JfyS1643-83-04). The original spore isolates picked from the FdU plates, as well as their respective one time spore-purified isolates, were analyzed by Southern analysis to insure correct excision from the genome. All analyzed strains had excised the cassette correctly. Fusarium venenatum JfyS1643-95-04 (Δtri5 ΔpyrG ΔamyA) was used as the strain for deletion of the Fusarium venenatum alkaline protease A gene (alpA).

### Example 27

### Construction of Plasmid pEJG69

The Microdochium nivale lactose oxidase (LOx) gene (SEQ ID NO: 71 for the DNA sequence and SEQ ID NO: 72 for the deduced amino acid sequence) was PCR amplified from pEJG33 (Xu et al., 2001, European Journal of Biochemistry 268: 1136-1142) using forward and reverse primers shown below.

Forward Primer:
(SEQ ID NO: 73)
5′-CCCGCATGCGTTCTGCATTTATCTTG-3′
Reverse Primer:
(SEQ ID NO: 74)
5′-GGGTTAATTAATTATTTGACAGGGCG-3′

The underlined portions represent introduced Sph I (forward) or Pac I (reverse) sites for cloning.

The PCR contained 200 μM dNTPs, 1 μM each primer, 50 ng of pEJG33, 1× Pwo buffer (Promega, Madison, Wis., USA), and 1 μl of Pwo Hot Start Polymerase (Promega, Madison, Wis., USA) in a final volume of 50 μl.

The amplification reaction was incubated in a ROBOCYCLER® programmed for 1 cycle at 95° C. for 2 minutes; 10 cycles each at 95° C. for 30 seconds, 55° C. for 45 seconds, and 72° C. for 1 minute; 20 cycles each at 95° C. for 30 seconds, 55° C. for 45 seconds, and 72° C. for 1 minutes with an additional 20 second extension for each subsequent cycle; and 1 cycle at 50° C. for 10 minutes.

PCR products were separated by 1% agarose gel electrophoresis using TAE buffer. A fragment of approximately 1.5 kb was excised from the gel and agarose-extracted using a QIAQUICK® Gel Extraction Kit.

The lactose oxidase gene was re-amplified using the same conditions and purified as described above, except that the polymerase and buffer were replaced with Taq DNA polymerase and Tao DNA Polymerase Buffer, respectively, and the gel-purified PCR product above was used as template. The PCR product was cloned into pCR®2.1 using a TOPO® TA Cloning Kit and sequenced to insure the absence of PCR errors. The resulting error-free plasmid was digested with Sph I, treated with T4 DNA polymerase (New England Biolabs Inc., Ipswich, Mass., USA), purified using a QIAQUICK® Nucleotide Removal Kit (QIAGEN Inc., Valencia, Calif., USA), and digested with Pac I. The fragment was purified by 1% agarose gel electrophoresis in TAE buffer, and a fragment of approximately 1.5 kb was excised from the gel and agarose-extracted using a QIAQUICK® Gel Extraction Kit.

Plasmid pEJG61 was digested with Bsp LU11I, treated with Klenow DNA polymerase (New England Biolabs Inc., Ipswich, Mass., USA) according to the manufacturer's directions, and then digested with Pac 1. The digested plasmid was purified by 1% agarose gel electrophoresis in TAE buffer and a 8 kb fragment was excised and agarose-extracted using a QIAQUICK® Gel Extraction Kit.

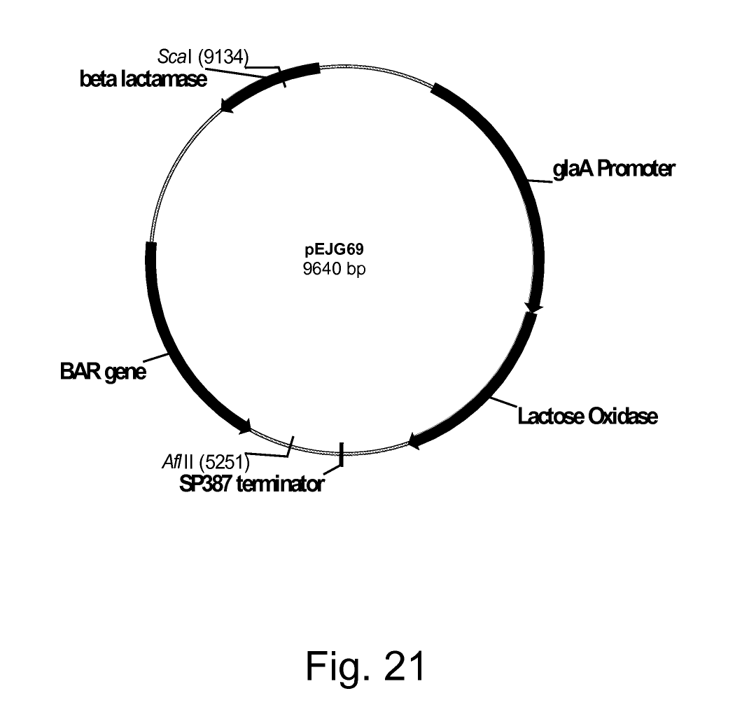

The LOx coding sequence was ligated to the Bsp LU11I- and PacI-digested pEJG61 using T4 DNA Ligase according to the manufacturer's directions. Plasmids were screened by sequence analysis to insure the absence of PCR errors and a resulting plasmid was identified and designated pEJG69 (FIG. 21).

### Example 28

### Construction of Plasmid pEJG65

Plasmid pEJG61 (Example 4) was digested with Bsp LU11I, treated with Klenow DNA polymerase, and digested with Pac I. The digested plasmid was isolated by 1% agarose gel electrophoresis in TAE buffer and a 8.1 kb fragment was excised and agarose-extracted using a QIAQUICK® Gel Extraction Kit.

The Candida antarctica lipase A coding sequence (SEQ ID NO: 75 for the DNA sequence and SEQ ID NO: 76 for the deduced amino acid sequence) was PCR amplified from pMT1229 (WO 94/01541) using forward and reverse primers shown below.

| | |
|---|---|
| Forward primer: | |
| 5′-GCATGCGAGTGTCCTTGCGC-3′ | (SEQ ID NO: 77) |
| Reverse primer: | |
| 5′-TTAATTAACTAAGGTGGTGTGATG-3′ | (SEQ ID NO: 78) |

The PCR reaction contained 200 μM dNTPs, 1 μM each primer, 20 ng of pMT1229, 1× Pwo buffer (Promega, Madison, Wis., USA), and 1 μl of Pwo Hot Start Polymerase (Promega, Madison, Wis., USA).

The amplification reaction was incubated in a ROBOCYCLER® programmed for 1 cycle at 94° C. for 2 minutes; 10 cycles each at 94° C. for 30 seconds, 55° C. for 45 seconds, and 72° C. for 1 minute; 17 cycles each at 94° C. for 30 seconds, 55° C. for 45 seconds, and 72° C. for 1 minutes with an additional 20 second extension for each subsequent cycle; and 1 cycle at 72° C. for 10 minutes.

PCR products were isolated by 1% agarose gel electrophoresis in TAE buffer and a 1.4 kb fragment was excised and agarose extracted using a QIAQUICK® Gel Extraction Kit. The PCR fragment was cloned into pCR®2.1 using a TOPO® TA Cloning Kit and sequenced to verify the absence of PCR errors.

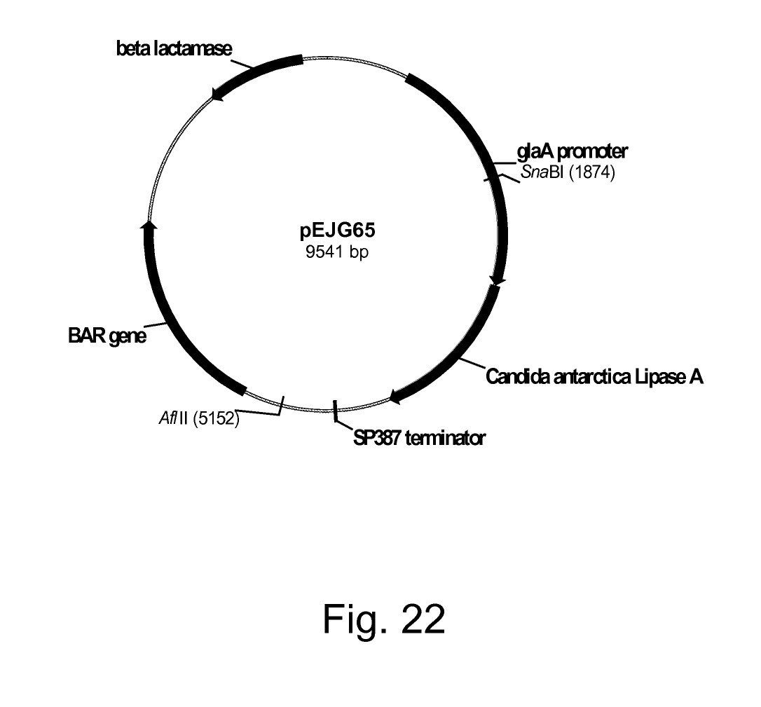

Due to the presence of an internal Sph I site in the coding sequence of the gene, the Candida antarctica lipase A coding sequence was liberated from pCR®2.1 as two separate fragments by separate digestions. To liberate the first fragment (1 kb), the plasmid was digested with Sph I and treated with T4 DNA polymerase. The polymerase was heat-inactivated for 10 minutes at 75° C. and the plasmid was digested with Nhe I. The second fragment (0.4 kb) was liberated from the plasmid with a Nhe I/Pac I digestion. Both digestions were subjected to 1% agarose gel electrophoresis in TAE buffer and a 1 kb fragment from the Sph I/Nhe I digestion and a 0.4 kb fragment from the Nhe I/Pac I digestion were excised and agarose-extracted using a QIAQUICK® Gel Extraction Kit. The two fragments were ligated to digested pEJG61 using T4 DNA ligase. The ligation reaction contained 1× Ligation Buffer (New England Biolabs Inc., Ipswich, Mass., USA), 100 ng of the 1 kb fragment above, 50 ng of the 0.4 kb fragment, 50 ng of digested pEJG61, and 10 units of T4 DNA ligase. The reaction was incubated at room temperature for 16 hours and used to transform E. coli XL10-GOLD® Ultra-competent cells according to manufacturer's instructions. Transformants were screened by sequence analysis and one clone containing a plasmid with the desired error-free coding sequence was identified and designated pEJG65 (FIG. 22).

### Example 29

### Construction of Plasmid pMStr19

Plasmid pMStr19 was constructed by cloning a Fusarium oxysporum phospholipase gene from pA2Ph10 (WO 1998/26057) into the Fusarium venenatum expression vector pDM181 (WO 2000/56900). PCR amplification was used to isolate the phospholipase gene on a convenient DNA fragment.

The Fusarium oxysporum phospholipase gene was specifically amplified from pA2Ph10 using standard amplification conditions with Pwo DNA polymerase (Roche Molecular Biochemicals, Basel, Switzerland) and an annealing temperature of 45° C. with the primers shown below.

PLMStr10:
(SEQ ID NO: 79)
5′-TCAGATTTAAATATGCTTCTTCTACCACTCC-3′
SwaI
PLMStr11:
(SEQ ID NO: 80)
5′-AGTCTTAATTAAAGCTAGTGAATGAAAT-3′

The resulting DNA fragment was gel-purified and digested with Swa I. Plasmid pDM181 was also digested with Swa I and dephosphorylated. The DNA fragments were then ligated together to produce plasmid pMStr18.

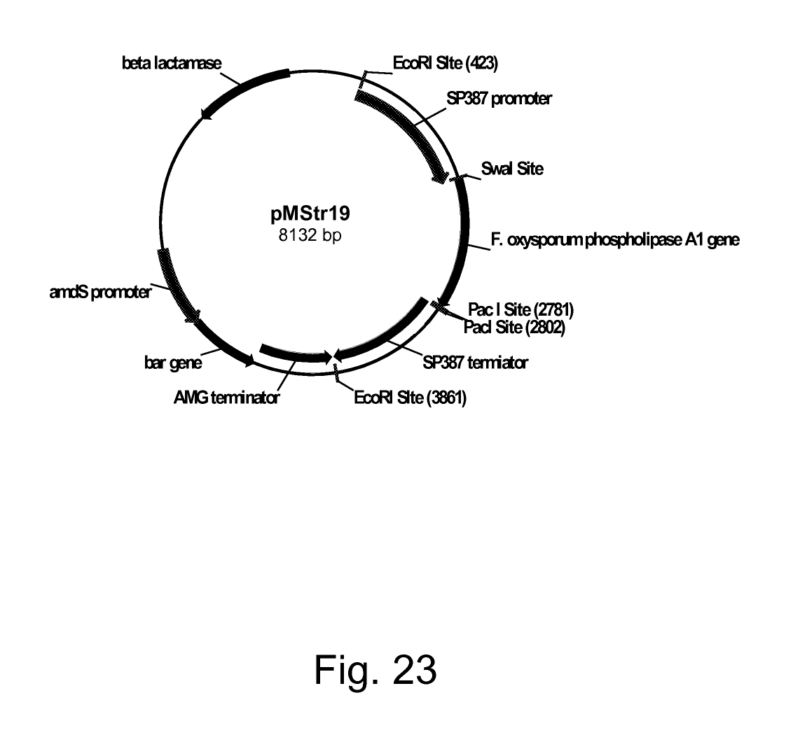

The phospholipase gene in two individual E. coli transformants of pMStr18, #4 and #17, generated using the ligation mixture, were sequenced using standard primer walking methods. Both had acquired single point mutations at different positions in the gene. The mutations were separated by a Nar I site, which cleaves pMStr18 twice. An error-free phospholipase gene was therefore assembled in the Fusarium expression vector pDM181 by digesting both pMStr18#4 and pMStr18#17 with Nar I, isolating the error-free fragments, and ligating them together to produce pMStr19 (FIG. 23). The phospholipase sequence in pMStr19 was confirmed using standard methods.

### Example 30

### Construction of Plasmid pEJG49

The Fusarium venenatum expression vector pEJG49 was generated by modification of pSheB1 (WO 2000/56900). The modifications included (a) removal of one Bsp LU11I site within the pSheB1 sequence by site-directed mutagenesis; (b) removal of 850 by of the Fusarium oxysporum trypsin promoter; (c) introduction of a Bsp LU11I site, by ligation of a linker, to aid in the insertion of the 2 kb Fusarium venenatum glucoamylase promoter; and (d) introduction of a Fusarium oxysporum phospholipase gene.

Removal of the Bsp LU11I site within the pSheB1 sequence was accomplished using a QUIKCHANGE™ Site-Directed Mutagenesis Kit according to the manufacturer's instructions with the following pairs of mutagenesis primers:

(SEQ ID NO: 81)
5′-GCAGGAAAGAACAAGTGAGCAAAAGGC-3′
(SEQ ID NO: 82)
5′-GCCTTTTGCTCACTTGTTCTTTCCTGC-3′

This created pSheB1 intermediate 1.

Removal of 930 bp of the Fusarium oxysporum trypsin promoter was accomplished by digesting pSheB1 intermediate 1 (6,971 bp) with Stu I and Pac I, subjecting the digest to 1% agarose gel electrophoresis using TBE buffer, excising the 6,040 bp vector fragment, and purifying the excised fragment with a QIAQUICK® Gel Extraction Kit. To introduce a new Bsp LU11I site, a linker was created using the following primers:

| | |
|---|---|
| 5′-dCCTACATGTTTAAT-3′ | (SEQ ID NO: 83) |
| Bsp Lu11I | |
| 5′-dTAAACATGTAGG-3′ | (SEQ ID NO: 84) |

Each primer (2 μg each) was heated at 70° C. for 10 minutes and then cooled to room temperature over an hour. This linker was ligated into the Stu 1-Pac 1-digested pSheB1 intermediate 1 vector fragment, creating pSheB1 intermediate 2. Vector pSheB1 intermediate 2 was then digested with Bsp Lu11I and Pac I. The digested vector was purified by 1% agarose gel electrophoresis in TBE buffer, excised from the gel, and agarose-extracted using a QIAQUICK® Gel Extraction Kit.

The Fusarium oxysporum phospholipase gene fragment was also generated by PCR using pMSTR19 as template. The following PCR primers were used to introduce a Sph I site at the 5′ end and a Pac I site at the 3′ end of the gene:

| | |
|---|---|
| 5′-GGGGGCATGCTTCTTCTACCACTCC-3′ | (SEQ ID NO: 85) |
| Sph I | |
| 5′-GGGGTTAATTAAGAGCGGGCCTGGTTA-3′ | (SEQ ID NO: 86) |
| Pac I | |

The conditions for PCR and purification were performed as above. The phospholipase gene fragment was cloned into pCR®-TOPO® according to the manufacturer's instructions. The pCR®-TOPO® phospholipase clone was then digested with Sph I and treated with T4 DNA polymerase to remove the protruding 3′ termini. The fragment was purified using QIAQUICK® Nucleotide Removal Kit and digested with Pac I. The digestion was purified by 1% agarose gel electrophoresis in TBE buffer and a 1 kb band was excised from the gel and purified using a QIAQUICK® Gel Extraction Kit.

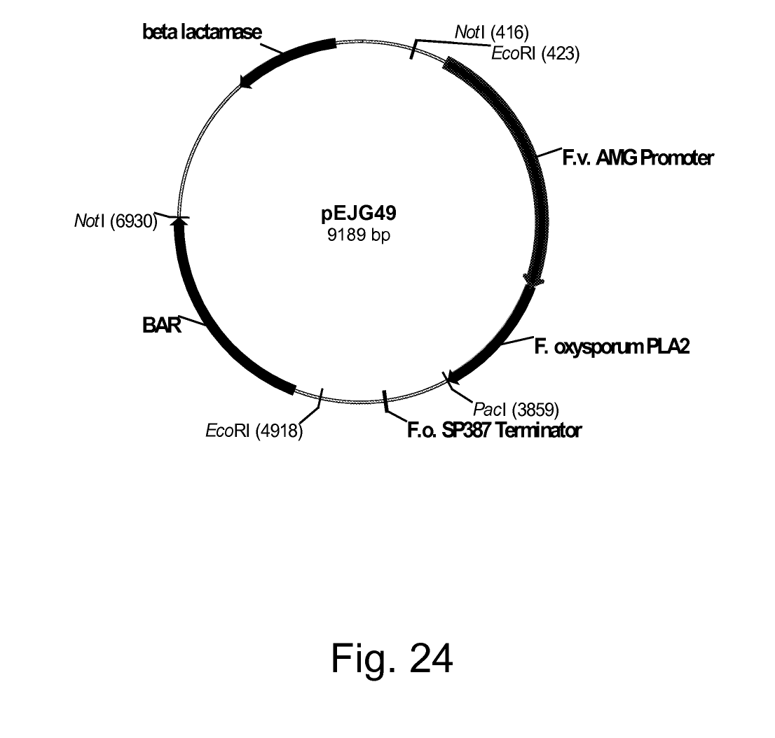

Plasmid pSheb1 intermediate 2 (above) was digested with Stu I and Bsp Lu11I and purified using a QIAQUICK® Nucleotide Removal Kit. The fragment was then ligated to a 2 kb Stu I-Bsp Lu11I Fusarium venenatum glucoamylase promoter fragment (WO 2000/056900). This vector, known as pSheb1 intermediate 3, was digested with Bsp Lu11I, treated with Klenow fragment to fill in the 5′ overhang, digested with Pac I, and purified using a QIAQUICK® Nucleotide Removal Kit. The fragment was then ligated to the Sph I, blunt-Pac I Fusarium oxysporum phospholipase fragment (described above). The resulting vector, designated pEJG49 (FIG. 24), harbored the phospholipase reporter gene under the transcriptional control of the Fusarium venenatum glucoamylase promoter.

### Example 31

### Construction of Plasmid pEmY15

Site-directed mutagenesis was used to remove one of each of the Eco RI and Not I restriction sites from expression plasmid pEJG49 and render these restriction sites flanking the bialaphos resistance marker (bar gene) unique. The mutagenesis was completed using forward and reverse primers shown below and a QUIKCHANGE® Site-Directed Mutagenesis Kit.

Forward primer:
(SEQ ID NO: 87)
5′-cctgcatggccgcCgccgcCaattcttacaaaccttcaaca
gtgg-3′
Reverse primer:
(SEQ ID NO: 88)
5′-ccactgttgaaggtttgtaagaattGgcggcGgcggccatg
cagg-3′

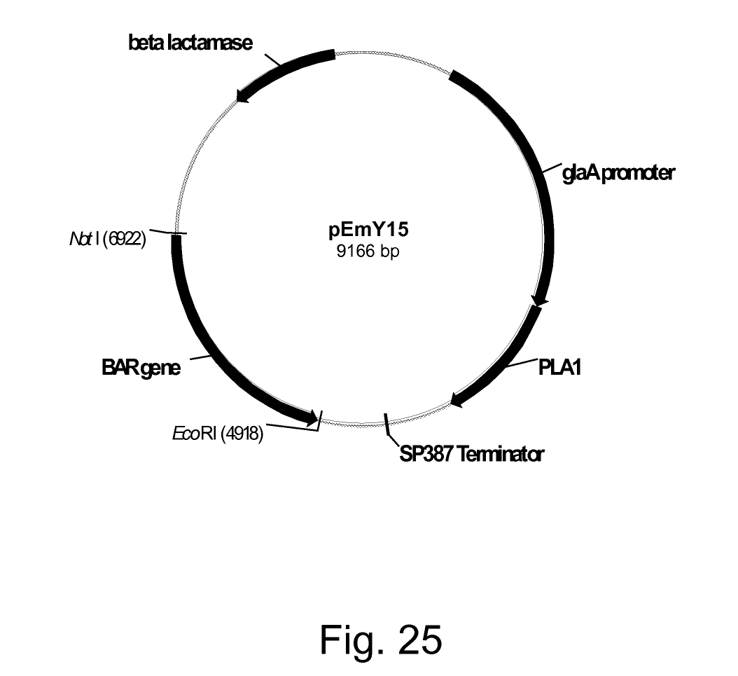

The uppercase letters indicate the desired changes and the resulting plasmid was designated pEmY15 (FIG. 25).

### Example 32

### Construction of Plasmid pEmY24

In order to replace the bar gene in expression plasmid pEmY15 with the Fusarium venenatum pyrG gene, the following protocol was performed. Plasmid pEmY15 was digested with Eco RI and Not I and purified by 1% agarose gel electrophoresis in TAE buffer. A 7.1 kb fragment was excised and agarose extracted using a QIAQUICK® Gel Extraction Kit.

A 2.3 kb fragment of the pyrG gene was PCR amplified from pDM156.2 using forward and reverse primers shown below.

Forward primer:
(SEQ ID NO: 89)
5′-ATAAGAATgcggccgcTCCAAGGAATAGAATCACT-3′
Reverse primer:
(SEQ ID NO: 90)
5′-CGgaattcTGTCGTCGAATACTAAC-3′

The bold sequence corresponds to an introduced Not I site and Eco RI site for the forward and reverse primers, respectively.

The amplification reaction was composed of 1× ThermoPol Buffer, 200 μM dNTPs, 31 ng of pDM156.2, 1 μM each primer, and 1 unit of VENT® DNA polymerase in a final volume of 50 μl.

The reaction was incubated in an EPPENDORF® MASTERCYCLER® programmed for 1 cycle at 95° C. for 3 minutes; 30 cycles each at 95° C. for 30 seconds, 55° C. for 1 minute; and 72° C. for 3 minutes; and 1 cycle at 72° C. for 7 minutes.

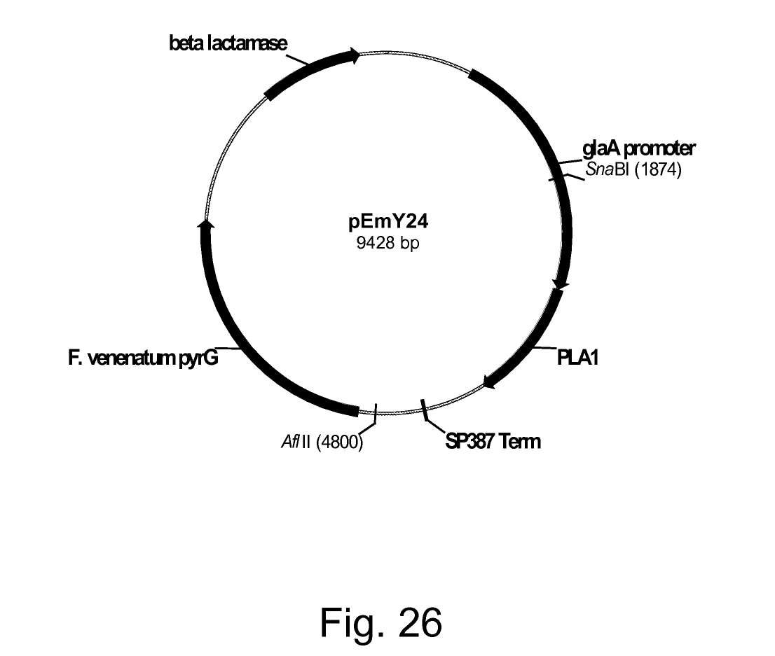

PCR products were isolated by 1% agarose gel electrophoresis in TAE buffer and a 2.3 kb fragment was excised and agarose-extracted using a MINELUTE® Gel Extraction Kit. The fragment was then digested with Eco RI and Not I and the digestion reaction purified using a MINELUTE® Reaction Cleanup Kit. The fragment was ligated to Not I/Eco RI-digested pEmY15 using T4 DNA ligase according to the manufacturer's instructions. The ligation mixture was transformed into E. coli XL1-Blue sub-cloning-grade competent cells (Stratagene, La Jolla, Calif., USA) according to the manufacturer's instructions. Transformants were sequenced to insure the absence of PCR errors and a plasmid was identified containing an error-free pyrG fragment. The resulting plasmid was designated pEmY24 (FIG. 26).

### Example 33

### Construction of Plasmid pDM257

Plasmid pEmY24 (Example 32) was digested with Afl II and Sna BI. A 6.5 kb fragment was purified by 1% agarose gel electrophoresis in TAE buffer, excised from the gel, and agarose-extracted using a QIAQUICK® Gel Extraction Kit. Plasmid pEJG65 was digested with Afl II and Sna BI. A 3.3 kb fragment was purified by 1% agarose gel electrophoresis in TAE buffer, excised from the gel, and agarose-extracted using a QIAQUICK® Gel Extraction Kit.

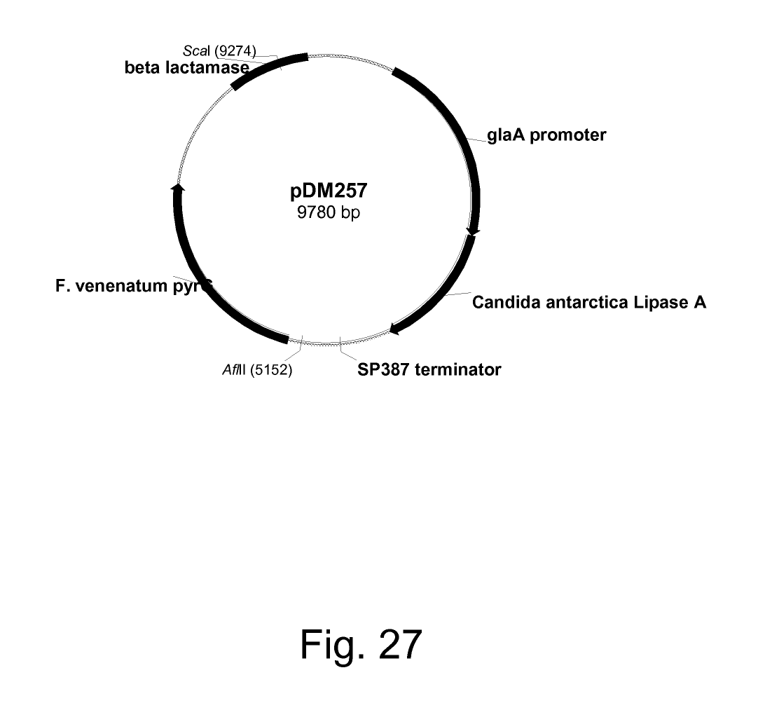

The two fragments were ligated together using T4 DNA ligase according to the manufacturer's instructions. The ligation mixture was transformed into E. coli XL1-Blue sub-cloning-grade competent cells according to the manufacturer's instructions. Transformants were screened by sequence analysis and a clone was identified containing a plasmid with the desired fragments. The resulting plasmid was designated pDM257 (FIG. 27).

### Example 34

### Construction of Plasmid pDM258

Plasmid pDM257 was digested with Sca I and Afl II and purified by 1% agarose gel electrophoresis in TAE buffer and a 4.1 kb fragment was excised from the gel and agarose-extracted using a QIAQUICK® Gel Extraction Kit. Plasmid pEJG69 was also digested with Sca I and Afl II and purified by 1% agarose gel electrophoresis in TAE buffer and a 5.8 kb fragment was excised from the gel and agarose-extracted as above.

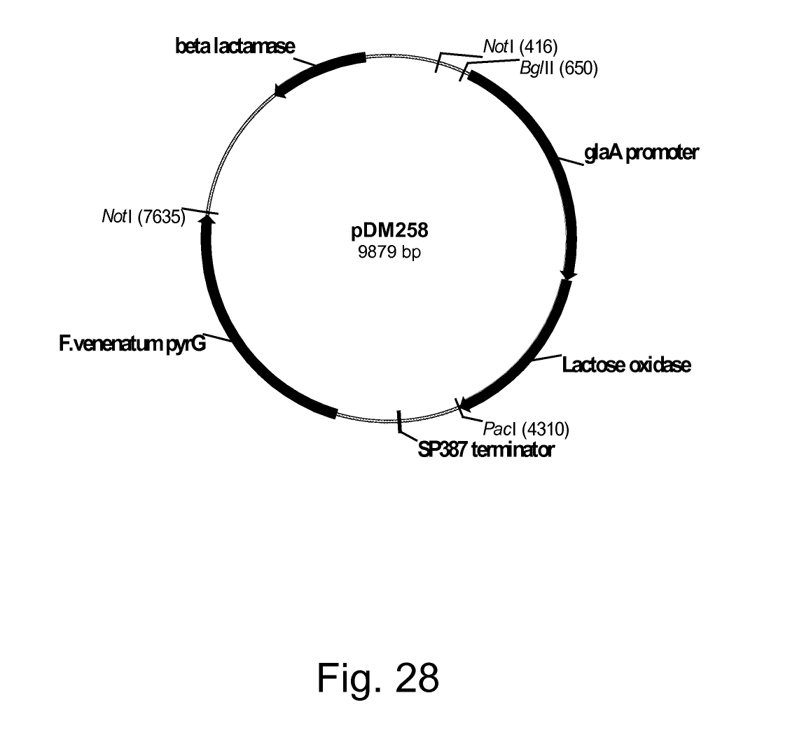

The two fragments were ligated together using T4 DNA ligase according to the manufacturer's instructions. The ligation mixture was transformed into E. coli XL1-Blue sub-cloning-grade competent cells according to the manufacturer's instructions. Transformants were screened by sequence analysis and the desired plasmid was identified and designated pDM258 (FIG. 28).

### Example 35

### Expression of Lactose Oxidase in Fusarium venenatum Strain JfyS1643-95-04

Protoplasts of Fusarium venenatum JfyS1643-95-04 (Δtri5 ΔpyrG ΔamyA) were generated as described in Example 5. The protoplasts were then transformed according to the procedure described in Example 20 with pDM258, harboring the Microdochium nivale lactose oxidase expression vector, to evaluate the expression potential of the Fusarium venenatum JfyS1643-95-04 strain. Transformants were grown in shake flasks as described in Example 21 except that the flasks were incubated for five days at 28° C. with shaking at 200 rpm.

The shake flask broths were assayed for lactose oxidase activity using an activity assay in conjunction with a BIOMEK® 3000, (Beckman Coulter, Inc, Fullerton, Calif., USA). The lactose oxidase assay was a modified version of the Glucose Oxidase Assay Procedure (K-Glox) (Megazyme, Wicklow, Ireland). Culture supernatants were diluted appropriately in 0.1 M MOPS buffer pH 7.0 (sample buffer) followed by a series dilution from O-fold to 1/3-fold to 1/9-fold of the diluted sample. A lactose oxidase standard (Novozymes A/S, Bagsvaerd, Denmark) was diluted using 2-fold steps starting with a 0.056 mg/ml concentration and ending with a 0.007 mg/ml concentration in the sample buffer. A total of 20 μl of each dilution including standard was transferred to a 96-well flat bottom plate. One hundred microliters of a POD solution (Peroxidase, 4AA, stabilizers in potassium phosphate buffer pH 7 plus p-hydroxybenzoic acid and sodium azide) were added to each well followed by addition of 100 μl of glucose substrate (0.5 M glucose in sample buffer). The rate of reaction was measured at ambient temperature (approximately 26° C.) at 510 nm for a total of 10 minutes. Sample concentrations were determined by extrapolation from a standard curve generated using lactose oxidase as a standard. The highest producing lactose oxidase transformants were selected for growth and analysis in 2 liter fermenters.

The fermentation medium (pH 6) was composed per liter of 20 g of soya flour, 20 g of sucrose, 2.0 g of MgSO₄.7H₂O, 2.0 g of anhydrous KH₂PO₄, 2.0 g of K₂SO₄, 5.0 g of (NH₄)₂SO₄, 1.0 g of citric acid, 0.5 ml of 200×AMG trace metals solution (no nickel), and 0.5 ml of pluronic acid with a 20% maltose feed. The fermentations were run at 29.0+/−1.0° C., 1200 rpm, and 1.0 vvm aeration where % DO was maintained above 30%.

Fermentation broths were assayed for alpha-amylase activity using an Alpha-Amylase Assay Kit (Megazyme International Ireland Ltd., Wicklow, Ireland) in conjunction with a BIOMEK® 3000 and BIOMEK® NX (Beckman Coulter, Inc, Fullerton Calif., USA). Fermentation broths were assayed for lactose oxidase activity as described above.

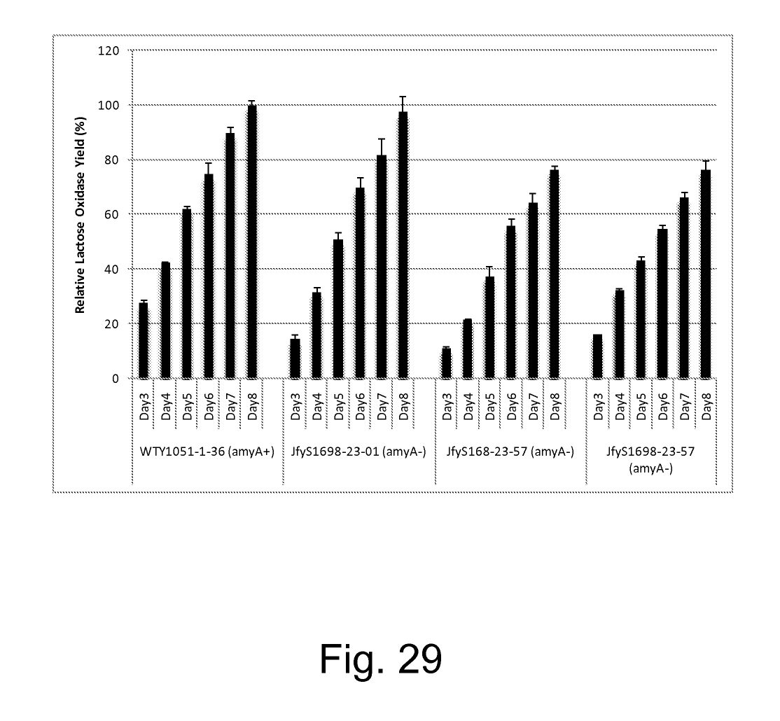

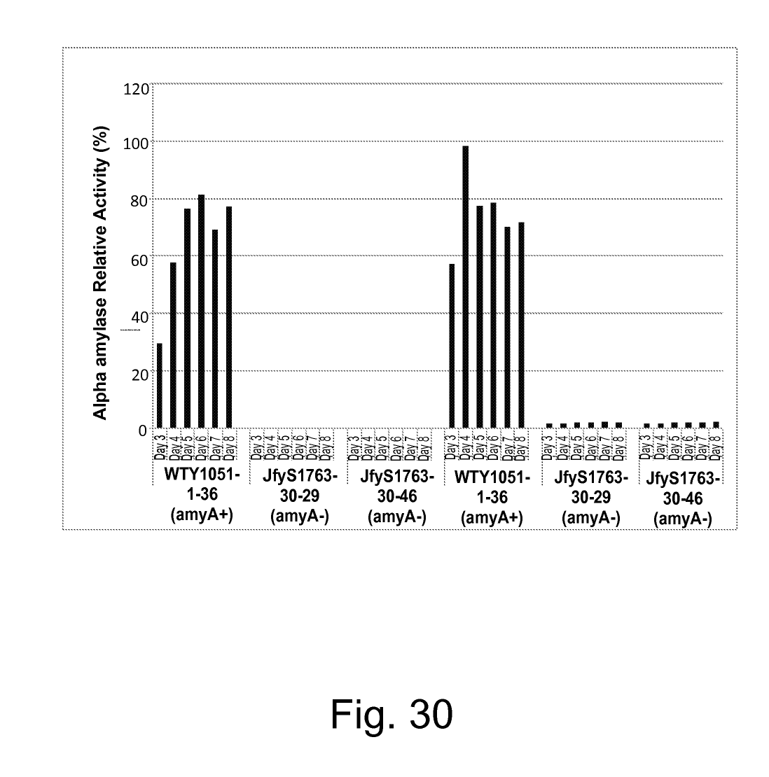

The resulting top transformant, Fusarium venenatum JfyS1643-95-04, had equivalent lactose oxidase production levels to other Fusarium venenatum transformants without the deletions in 2 liter fermenters (FIG. 29) indicating that deletion of the amyA gene did not have a negative impact on heterologous protein production. The deletion did, however, abolish alpha-amylase activity in the culture broth of this strain and all later strains in this lineage (FIG. 30). Since this transformant had equivalent heterologous protein production capacity to the current production strain, and reduced alpha-amylase levels during fermentation, Fusarium venenatum JfyS1643-95-04 host strain was selected for deletion of an alkaline protease A gene (alpA).

### Example 36

### Generation of the Fusarium venenatum Alkaline Protease A (alpA) Deletion Vector pJfyS1698-72-10

Upstream flanking sequence for use in the complete removal of the Fusarium venenatum A3/5 alkaline protease A (alpA) gene (SEQ ID NO: 91 for the DNA sequence and SEQ ID NO: 92 for the deduced amino acid sequence) was obtained using a GENOME WALKER™ Universal Kit. Each library generated with the kit was subjected to two rounds of PCR for the 5′ flanking sequence using a 5′ gene-specific primer and a 5′ nested primer shown below.

5′ gene-specific primer:
(SEQ ID NO: 93)
5′-GAGGAATTGGATTTGGATGTGTGTGGAATA-3′
5′ nested primer:
(SEQ ID NO: 94)
5′-GGAGTCTTTGTTCCAATGTGCTCGTTGA-3′

Sequence information was obtained from the PCR product using a Nested Adaptor Primer supplied with the BD GENOME WALKER™ Universal Kit and the 5′ nested primer above. The obtained sequence was used to design primers to amplify a 1 kb region of the 5′ alpA flanking sequence for insertion into the empty deletion vector pJfyS1579-41-11

The alpA 5′ flanking sequence was PCR amplified from Fusarium venenatum A3/5 genomic DNA using region-specific forward and reverse primers shown below. The underlined letters represent a Not I site, for later removal of the pCR®2.1 portion of the vector, and the italicized letters represent an Asc I site for vector cloning.

Forward primer:
(SEQ ID NO: 95)
5′-aaaaaaggcgcgccgcggccgcGTTACGGTGTTCAAGTACATCTTA
CA-3′
Reverse primer:
(SEQ ID NO: 96)
5′-aaaaaaggcgcgccATTGCTATCATCAACTGCCTTTCTT-3′

The amplification reaction contained 1× HERCULASE® Reaction Buffer, 120 ng of genomic DNA, 400 nm primers, 200 μM dNTPs, and 2.5 units of HERCULASE® DNA polymerase.

The amplification reaction was incubated in an EPPENDORF® MASTERCYCLER® programmed for 1 cycle at 95° C. for 2 minutes; 20 cycles each at 94° C. for 30 seconds, 56° C. for 30 seconds, and 72° C. for 1 minute 10 seconds; and 1 cycle at 72° C. for 7 minutes.

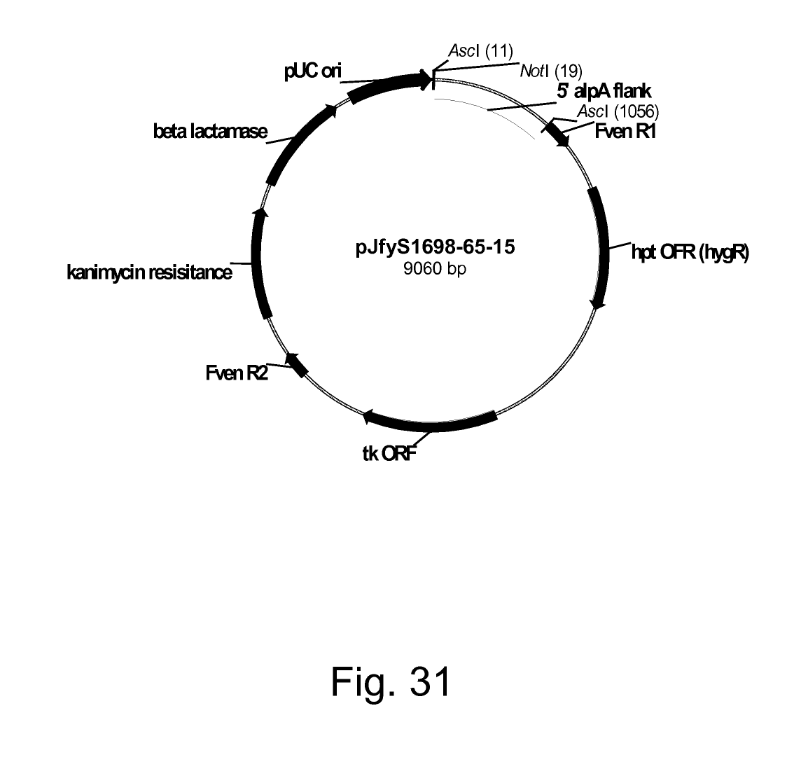

A 5 μl portion of the amplified reaction was visualized by 1% agarose gel electrophoresis using TAE buffer to insure the reaction had produced the desired 1 kb band. The insert was then directly cloned into pCR®2.1 TOPO® from the amplification reaction using a TOPO® TA Cloning Kit according to the manufacturer's instructions. Transformants were screened by restriction analysis with Eco RI to insure the presence of the insert and 5 correct preparations were combined. The insert was liberated from pCR®2.1 by digestion with Asc I and the fragment was purified by agarose gel electrophoresis as described above. The insert was cloned into Asc I-linearized pJfyS1579-41-11 using a QUICK LIGATION™ Kit and the ligation mixture used to transform E. coli SURE® chemically competent cells according to the manufacturer's protocol. Transformants were screened by sequence analysis to insure the absence of PCR errors. One plasmid containing the flanking sequence without errors was designated pJfyS1698-65-15 (FIG. 31) and used to insert the 3′ flanking sequence.

The 3′ flanking sequence of the alpA gene was amplified from Fusarium venenatum A3/5 genomic DNA using region specific forward and reverse primers shown below. The underlined letters represent a Not I site, for later beta-lactamase removal, and the italicized letters represent a Sbf I site for vector cloning.

Forward primer:
(SEQ ID NO: 97)
5′-aaaaacctgcaggGGATGTGTGTGGAATAGGATATG-3′
Reverse primer:
(SEQ ID NO: 98)
5′-aaaaacctgcagggcggccgcCCTCAAGGTGGAGAAATA
ATCTGT-3′

The PCR reaction contained 1× HERCULASE® Reaction Buffer, 120 ng of genomic DNA template, 400 nm primers, 200 μM dNTPs, and 2.5 units of HERCULASE® DNA polymerase.

The amplification reaction was incubated in an EPPENDORF® MASTERCYCLER® programmed for 1 cycle at 95° C. for 2 minutes; 20 cycles each at 94° C. for 30 seconds, 56° C. for 30 seconds, and 72° C. for 1 minute 10 seconds; and 1 cycle at 72° C. for 7 minutes.

A 5 μl portion of the amplified reaction was visualized on a 1% agarose gel in TAE buffer to insure the reaction had produced the desired 1 kb band. The 1 kb insert, directly from the PCR reaction, was then cloned into pCR®2.1 TOPO® using a TOPO® TA Cloning Kit. The resulting plasmid was sequenced to identify a colony containing the correct sequence. The fragment was then liberated from this plasmid by Sbf I digestion and purified by 1% agarose gel electrophoresis in TAE buffer. A 1 kb band was excised and agarose-extracted using a MINELUTE® Gel Extraction Kit.

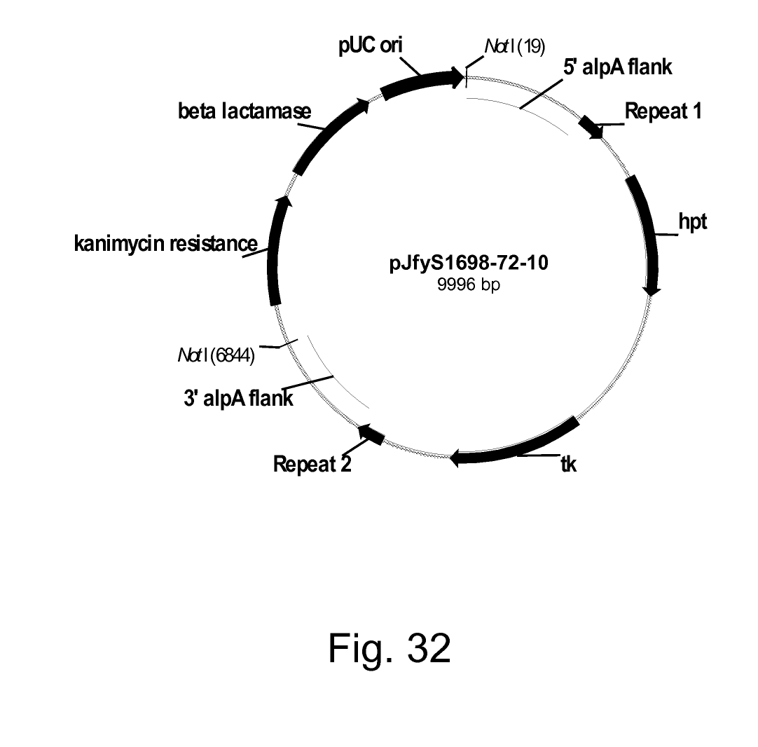

This fragment was then ligated to Sbf I linearized pJfyS1698-65-15 (treated with calf intestine phosphatase) using a QUICK LIGATION™ Kit and the ligation mixture was used to transform E. coli SURE® chemically competent cells according to the manufacturer's instructions. Transformants were screened by restriction analysis with Not I to insure the fragment had been inserted in the correct orientation and sequenced to insure no deviations from the expected sequence. The resulting plasmid pJfyS1698-72-10 (FIG. 32) was used for deletion of the alpA gene.

### Example 37

### Generation of Δtri5 ΔpyrG ΔamyΔ AalpA Fusarium venenatum Strain JfyS1763-11-01

Three transformants of Fusarium venenatum JfyS1643-95-04 (Δtri5 ΔpyrG ΔamyA) (Example 26) transformed with Not I-digested and gel-purified pJfyS1698-72-10 according to the procedure described in Example 20 were transferred from transformation plates with sterile toothpicks to new plates containing VNO₃RLMT medium supplemented with 125 μg of hygromycin B per ml and 10 mM uridine and incubated at room temperature for 7 days. For Southern analysis, 2 μg of Fusarium venenatum genomic DNA from each of the 3 transformants were digested with 34 units of Sph I. A DIG probe to the 5′ flanking sequence of the alpA gene was generated according to the method described in Example 21 using the forward and reverse primers shown below.

Forward primer:
(SEQ ID NO: 99)
5′-GCACGTTAGGCTCAAGCCAGCAAGG-3′
Reverse primer:
(SEQ ID NO: 100)
5′-GAGGCTCATGGATGTGGCGTTAATG-3′

Southern analysis performed as described in Example 21 indicated that one of the three transformants contained a single copy of the deletion cassette at the alpA gene locus and this transformant was designated Fusarium venenatum JfyS1698-83-2.

Fusarium venenatum JfyS1698-83-2 was sporulated as described in Example 5 and 10⁵ spores were plated onto a 150 mm diameter plate containing VNO₃RLMT medium supplemented with 50 μM FdU and 0.1 mM uridine. Spore isolates obtained were sub-cultured to a new plate containing VNO₃RLMT medium supplemented with 10 μM FdU and 0.1 mM uridine. The resulting spore isolates were analyzed by Southern analysis as described in Example 21 and one spore isolate was identified that had correctly excised the cassette. The isolate was designated Fusarium venenatum JfyS1698-94-04. Fusarium venenatum JfyS1698-94-04 was spore-purified once as described in Example 21 and one spore isolate was picked and designated Fusarium venenatum JfyS1763-11-01 (Δtri5 ΔpyrG ΔamyΔ AalpA).

Protoplasts of Fusarium venenatum JfyS1763-11-01 were generated and transformed as described in Examples 5 and 20 with pDM258. Transformants were analyzed as described in Example 35 and fermentation broths were assayed for alkaline protease activity. A PROTAZYME® AK tablet (Megazyme, Wicklow, Ireland) was suspended in 2.0 ml of 0.01% TRITON® X-100 by gentle stirring. Five hundred microliters of this suspension and 500 μl of assay buffer supplied with the PROTAZYME® AK tablet were mixed in an EPPENDORF® tube and placed on ice. Twenty microliters of protease sample (diluted in 0.01% TRITON® X-100) were added. The assay was initiated by transferring the EPPENDORF® tube to an EPPENDORF® thermomixer, which was set to the assay temperature. The tube was incubated for 15 minutes on the EPPENDORF® thermomixer at 1300 rpm. The incubation was stopped by transferring the tube back to an ice bath. Then the tube was centrifuged at 16,000×g in an ice cold centrifuge for a few minutes and 200 μl of supernatant was transferred to a microtiter plate. The absorbance at 650 nm was read as a measure of protease activity.

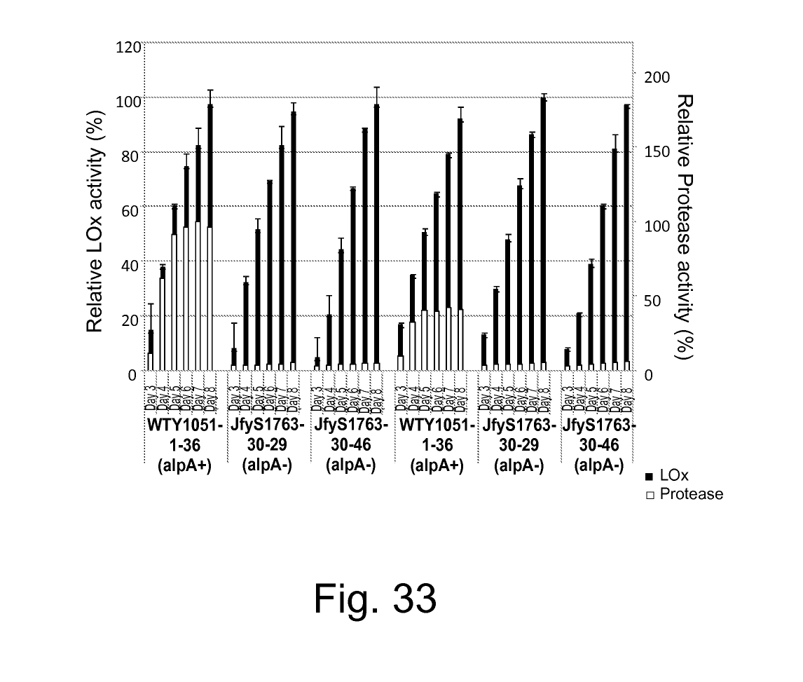

As with the amyA deletion, deletion of the alpA gene did not have a positive impact on lactose oxidase expression. However, the alkaline protease side activity in the fermentation supernatants was reduced 10-fold (FIG. 33).

### Example 38

### Generation of the dps1 Deletion Vector pJfyS111

The 3′ flanking sequence for the Fusarium venenatum depsipeptide synthase (dpsl) gene (SEQ ID NO: 101 for the DNA sequence and SEQ ID NO 102 for the deduced amino acid sequence) was PCR amplified from Fusarium venenatum JfyS1763-11-01 genomic DNA using the forward and reverse primers shown below. The underlined portion in the primer represents the introduced Sbf I site for cloning and the italicized portion corresponds to an introduced Not I site for later beta-lactamase removal. Genomic DNA was extracted using a DNEASY® Plant Maxi Kit.

Forward primer:
(SEQ ID NO: 103)
5′-GACTAAGCCCTGCAGGTTGGTCTCAATCGTCGCGACAG-3′
Reverse primer:
(SEQ ID NO: 104)
5′-AGTCTACCCCTGCAGGCGGCCGCTGGCATCGGTGGACGTAACACG
C-3′

The amplification reaction contained 1× HERCULASE® Reaction Buffer, 400 nM each primer, 200 μM dNTPs, 100 ng of genomic DNA, and 1.5 units of HERCULASE® DNA polymerase in a final volume of 50 μl. The amplification reaction was incubated in an EPPENDORF® MASTERCYCLER® programmed for 1 cycle at 95° C. for 2 minutes; 25 cycles each at 95° C. for 30 seconds, 57° C. for 30 seconds, and 72° C. for 1 minute and 20 seconds; and 1 cycle at 72° C. for 7 minutes.

The amplification reaction was purified using a MINELUTE® PCR Purification Kit.

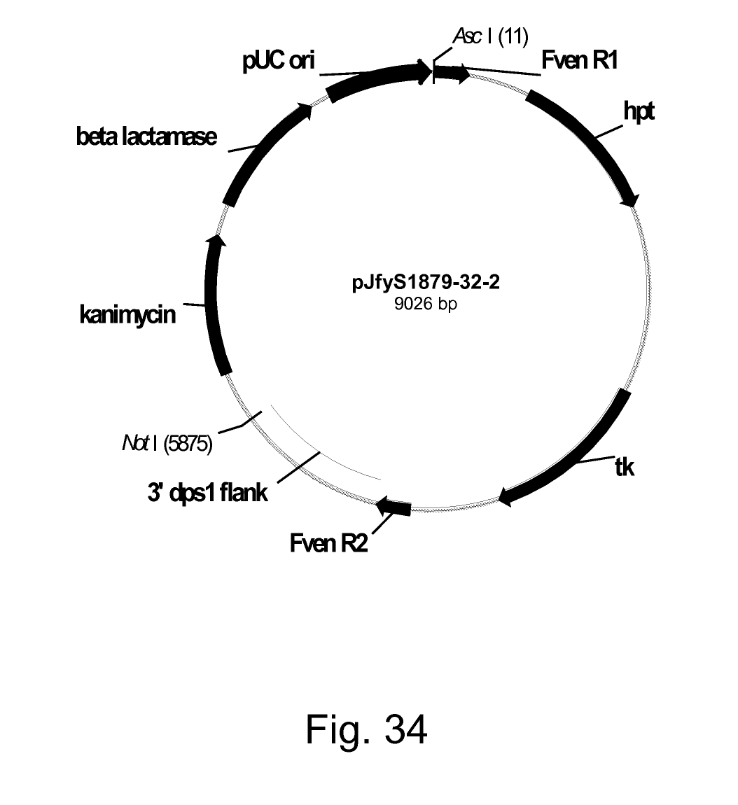

The purified reaction was then digested with Sbf I and submitted to 1% agarose gel electrophoresis using TAE buffer. A 1 kb band was excised from the gel and agarose-extracted using a MINELUTE® Gel Extraction Kit. The digested vector was then ligated to Sbf I-digested pJfyS1579-41-11 (Example 22) (which had been dephosphorylated with calf intestine phosphatase) using a QUICK LIGATION™ Kit according to the manufacturer's suggested protocols. Resulting clones were analyzed by restriction analysis with Eco RI (to check for insert presence and orientation) and sequence analysis (to insure the absence of PCR errors), and the resulting plasmid was designated pJfyS1879-32-2 (FIG. 34).

In order to obtain flanking sequence on the 5′ end of the dps1 gene, a GENOME WALKER™ Universal Kit was used as described in Example 36 with gene-specific and gene-specific nested primers shown below.

Gene-Specific primer:
(SEQ ID NO: 105)
5′-GCTATTGAGGGGACTATCTCCATGACTACA-3′
Gene-Specific nested primer:
(SEQ ID NO: 106)
5′-GCCTACCATCGACAGCAGTAAGATATTCC-3′

The 5′ dps1 flanking sequence was amplified from Fusarium venenatum JfyS1763-11-1 genomic DNA using forward and reverse primers indicated below. The underlined portion in the forward primer represents an introduced Asc I site for cloning and the italicized portion corresponds to an introduced Not I site for later beta-lactamase removal. The amplification reaction and cycling parameters were identical to those described above except the primers used were those below, the annealing temperature used was 53° C., and the extension time was 1 minute and 15 seconds.

Forward primer:
(SEQ ID NO: 107)
5′-ATGTGCTACAGGCGCGCCGCGGCCGCGAGTTCCAACATGTCTTATT
ATCC-3′
Reverse primer:
(SEQ ID NO: 108)
5′-TACTGTACCGGCGCGCCATCTGAGCCAAGAGACTCATTCAT-3′

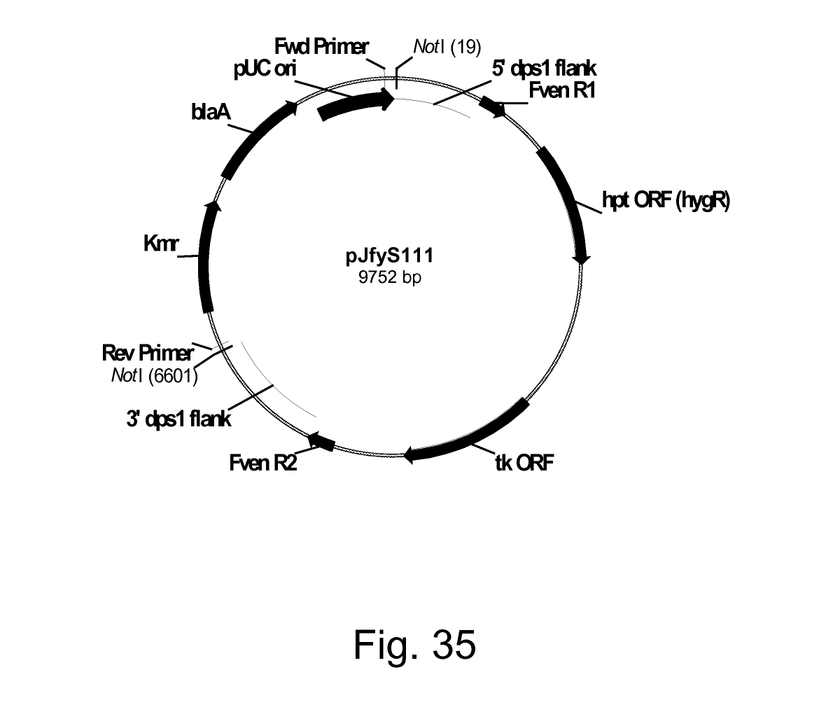

The PCR reaction was purified using a MINELUTE® PCR Purification Kit. The purified reaction was digested with Asc I, and subjected to 1% agarose gel electrophoresis using TAE buffer. A 0.7 kb band was excised from the gel and agarose-extracted as described above. The 0.7 kb band was ligated to pJfyS1879-32-2 (digested with Asc I and dephosphorylated with calf intestine phosphatase) using a QUICK LIGATION™ Kit. Resulting clones were analyzed by sequence analysis to insure the absence of PCR errors, and the resulting plasmid was designated pJfyS111 (FIG. 35) and used to delete the Fusarium venenatum dps1 gene.

### Example 39

### Generation of Δtri5 ΔpyrG ΔamyA ΔaIpA Δdps1 Fusarium venenatum Strain JfyS1879-57-01

When Fusarium venenatum JfyS1763-11-01 protoplasts were transformed with Not I-digested and gel-purified pJfyS111 according to the procedure described in Example 20, 77 transformants were obtained. Of those 48 were transferred from transformation plates with sterile toothpicks to new plates containing VNO₃RLMT medium supplemented with 125 μg of hygromycin B per ml and 10 mM uridine and incubated at room temperature for 7 days.

Fungal biomass was produced by inoculating 25 ml of M400 medium supplemented with 10 mM uridine with four 1 cm agar plugs from 7 day old transformants obtained as described in Example 21. The cultures were incubated for 3 days at 28° C. with shaking at 150 rpm. Agar plugs were removed and the cultures were filtered through MIRACLOTH™. Harvested biomass was frozen with liquid nitrogen and the mycelia were ground using a mortar and pestle.

Genomic DNA was isolated using a DNEASY® Plant Maxi Kit according to the manufacturer's instructions, except the lytic incubation period at 65° C. was extended to 1.5 hours from 10 minutes.

Two μg of genomic DNA were digested with 28 units each of Nco I and Spe I in a 50 μl reaction volume at 37° C. for 22 hours. The dig estion was subjected to 1.0% agarose gel electrophoresis in TAE buffer. The DNA was fragmented in the gel by treating with 0.25 M HCl, denatured with 1.5 M NaCl-0.5 M NaOH, neutralized with 1.5 M NaCl-1 M Tris pH 8, and then transferred in 20×SSC to a NYTRAN® Supercharge nylon membrane using a TURBOBLOTTER™ Kit. The DNA was UV cross-linked to the membrane using a UV STRATALINKER™ and pre-hybridized for 1 hour at 42° C. in 20 ml of DIG Easy Hyb.

A DIG probe to the 3′ flanking sequence of the dps1 gene was generated according to the method described in Example 21 using the forward and reverse primers shown below.

Forward primer:
(SEQ ID NO: 109)
5′-CTTGACTATTATCTCACGTTGTCAG-3′
Reverse primer:
(SEQ ID NO: 110)
5′-TCAAGTGTTGTGTAATGTTGGAACA-3′

Southern analysis performed as described in Example 21 indicated that three of the 8 transformants contained the deletion fragment in a single copy at the dpsl locus. One was named Fusarium venenatum JfyS1879-43-05.

Fusarium venenatum JfyS1879-43-05 was sporulated as described in Example 5 and 10⁵ spores were plated onto a 150 mm diameter plate containing VNO₃RLMT medium supplemented with 50 μM FdU and 0.1 mM uridine. Spore isolates obtained were sub-cultured to new plates containing VNO₃RLMT medium supplemented with 50 μM FdU and 0.1 mM uridine. The resulting spore isolates were analyzed by Southern analysis according to Example 21 and one spore isolate was identified that had correctly excised the cassette. The isolate was designated Fusarium venenatum JfyS1879-52-3. Fusarium venenatum JfyS1879-52-03 was spore purified once as described in Example 21 and one spore isolate was picked and designated Fusarium venenatum JfyS1879-57-01 (Δtri5 ΔpyrG ΔamyA ΔalpA Δcips1).

### Example 40

### Construction of Trichoderma reesei hemA Deletion Vector pJfyS120

In order to delete the Trichoderma reesei aminolevulinic acid synthase gene, the 3′ hemA flanking sequence was PCR amplified from Trichoderma reesei RutC30 genomic DNA using the forward and reverse primers shown below. The underlined portion in the primer represents the introduced Sbf I site for cloning and the bold portion corresponds to an introduced Not I site for later beta-lactamase removal.

Forward primer (#064877)
(SEQ ID NO: 111)
5′-TATAGCGTACCTGCAGGTGTCATGCCCGCGGCTTTGCCTTGA-3′
Reverse primer (#064878)
(SEQ ID NO: 112)
5'-ATGCTGTACCTGCAGGCGGCCGCCGCTCCCGATCATCATCCCTCCG
AG-3'

The amplification reaction was composed of 1× HERCULASE® Reaction Buffer, 400 nM each primer, 200 μM dNTPs, 125 ng of genomic DNA, and 1.5 units of HERCULASE® DNA polymerase. The reaction was incubated in an EPPENDORF® MASTERCYCLER® programmed for 1 cycle at 95° C. for 2 minutes; 25 cycles each at 95° C. for 30 seconds, 57° C. for 30 seconds, and 72° C. for 1 minute and 45 seconds; and 1 cycle at 72° C. for 7 minutes.

PCR products were separated by 1% agarose gel electrophoresis using TAE buffer. A fragment of approximately 1.5 kb was excised from the gel and agarose extracted using a MINIELUTE® Gel Extraction Kit.

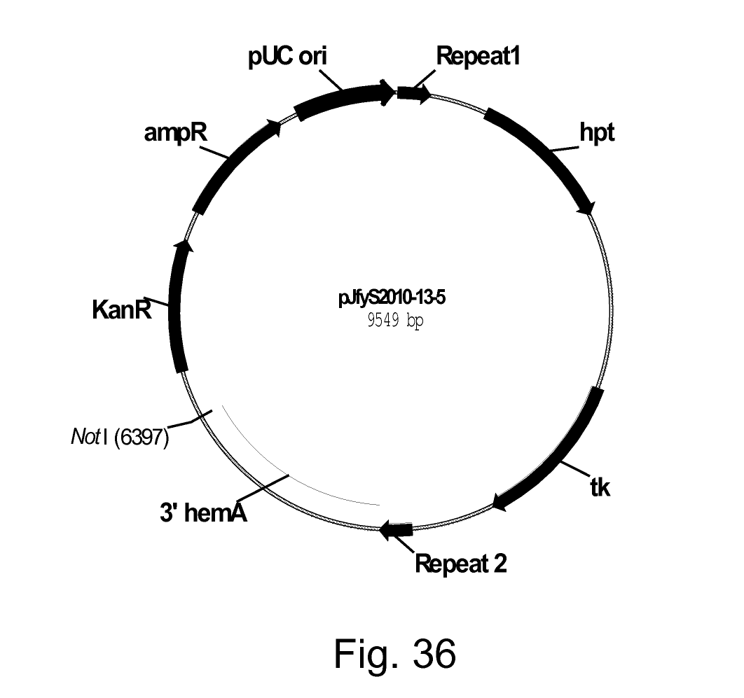

The 1.5 kb fragment was cloned into pCR®2.1 using a TOPO®-TA Cloning Kit according to the manufacturer and sequenced to ensure the absence of PCR errors. The fragment was liberated from pCR2.1 by Sbf I digestion and purified by 1% agarose gel electrophoresis in TAE buffer. The 1.5 kb band was excised and agarose extracted using a MINELUTE® Gel Extraction Kit. The digested fragment was ligated to the universal deletion vector pJfyS1579-41-11 (Example 22), which had been previously digested with Sbf I and calf intestine phosphatase dephosphorylated, using a QUICK LIGATION™ Kit according to the manufacturer. Resulting clones were analyzed by sequence analysis to check for insert presence and orientation and to ensure the absence of PCR errors. The resulting plasmid was named pJfyS2010-13-5 (FIG. 36).

The 5′ hemA flanking sequence was amplified from Trichoderma reesei RutC30 genomic DNA using the forward and reverse primers shown below. The underlined portion in the primer represents the introduced Asc I site for cloning, and the bold portion corresponds to an introduced Not I site for later beta lactamase removal.

Forward Primer (#065245):
(SEQ ID NO: 113)
5′-CATGGTTTAAACGGCGGCGCGCCGCGGCCGCAATTCAGAGCATC
ACGGTTGAGGGA-3′
Reverse Primer (#065246):
(SEQ ID NO: 114)
5′-CTTGTTTTGTCGGGCGCGCCACATGGCCTTGGATTGACGCAGGA
C-3′

The amplification reaction was performed to the same procedure above for the 3′ flanking sequence above. The reaction was incubated in an EPPENDORF®MASTERCYCLER® programmed for 1 cycle at 95° C. for 2 minute; 25 cycles each at 95° C. for 30 seconds, 53° C. for 30 seconds, and 72° C. for 1 minute and 15 seconds; and 1 cycle at 72° C. for 7 minutes.

PCR products were separated by 1% agarose gel electrophoresis using TAE buffer. A fragment of approximately 1 kb was excised from the gel and agarose extracted using a MINIELUTE® Gel Extraction Kit.

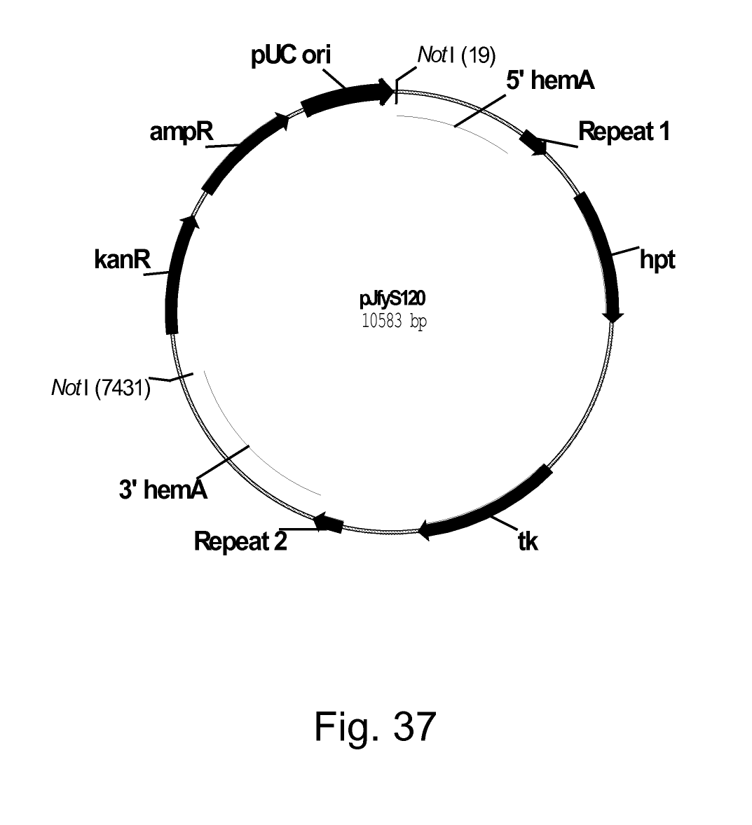

The 1 kb fragment was subsequently digested with Asc I and gel-purified as described above. The digested fragment was ligated to pJfyS2010-13-5, which had been previously digested with Sbf I and calf intestine phosphatase dephosphorylated, using a QUICK LIGATION™ Kit according to the manufacturer. Resulting clones were analyzed by sequence analysis to ensure the absence of PCR errors and the resulting plasmid was named pJfyS120 (FIG. 37). Plasmid pJfyS120 was used to delete the Trichoderma reesei hemA gene.

### Example 41

### Generation of Protoplasts of Trichoderma reesei strain RutC30

To generate a fresh culture of T. reesei strain RutC30, plugs were transferred from a stock containing plugs of the strain submerged in 10% glycerol, to a fresh PDA plate and incubated at 28° C. for 7 days. Spores were collected in 4 ml of 0.01% Tween® 20 using a sterile spreader and 350 μl of spores used to inoculate 25 ml of YPG_{2%} in a baffled shake flask and incubated 16 hours at 28° C. with shaking at 90 rpm. Mycelia were collected by filtering the culture through a MILLIPORE® STERICUP® 250 ml 0.2 μm filter unit collecting the gremlins on the filter. Mycelia were washed with approximately 100 ml of 1.2 M sorbitol. Mycelia were resuspended in 20 ml of protoplasting solution composed of 5 mg/ml GLUCANEX™ (Novozymes, Bagsvaerd, DK) in 1 M MgSO₄ and 0.36 units/ml chitinase (Sigma Aldrich, St Louis, Mo., USA). The protoplasting solution was incubated for 25 minutes in 125 ml shake flasks at 34° C. with shaking at 90 rpm. The reaction was stopped by incubating the flasks on ice. The protoplasts were transferred to a 50 ml conical bottomed tube (and 30 ml of ice cold 1.2 M sorbitol was added. The tube was centrifuged at 377×g in a Sorvall RT 6000B swinging-bucket centrifuge (Thermo-Fischer Scientific, Waltham, Mass., USA) for 10 minutes at room temperature (approximately 24-28° C.). The supernatant was discarded and protoplasts were washed with 30 ml of 1.2 M sorbitol. The tube centrifugation was repeated and supernatant discarded. The pellet was resuspended in 1.2 M sorbitol and a 10 μl sample was removed to determine the concentration of protoplasts using a hemacytometer (VWR, West Chester, Pa.). The tube containing the protoplasts was centrifuged at 377×g and the protoplasts were resuspended in TrSTC to a final concentration of 2×10⁸ protoplasts/ml.

### Example 42

### Deletion of the Trichoderma reesei Aminolevulinic Acid Synthase (hemA) Gene

Trichoderma reesei RutC30 protoplasts were transformed with Not I digested and gel-purified deletion vector pJfyS120 as described in Example 20 with the exceptions noted below. One hundred μl of protoplasts were transferred to a 14 ml polypropylene tube to which 2 μg of gel-purified pJfyS120 was added. Two hundred and fifty microliters of polyethylene glycol 4000 was added and the tubes were mixed gently by inverting 6 times. The tubes were incubated 34° C. for 30 minutes after which 3 ml of TrSTC was added. The tube contents were plated onto two 150 mm PDA plates containing 1 M sucrose and 5 mM aminolevulinic acid (ALA), which were incubated 28° C. for 16 hours. An overlay cooled to 50° C. containing PDA, 100 μg/ml hygromycin B, and 5 mM ALA was poured on top of the plates and allowed to cool at room temperature for 30 minutes. The plates were then incubated for 5 days at 28° C.

The transformation yielded 134 transformants. Each transformant was transferred to one well of a 6-well cell culture plate containing 5 ml of PDA with 5 mM ALA and 25 μg/ml hygromycing B and incubated at 28° C. for 5 days. Transformants were tested for ALA auxotrophy by scraping a small amount of spores from the transformant to a different 6-well plate containing TrMM medium without supplemented ALA. Three transformants displaying auxotrophy were then subcultured to a PDA plate containing 5 mM ALA and incubated 28° C. for 5 days. To generate genomic DNA for Southern analysis, four 1 cm² plugs of the 5 day old transformants were inoculated into 25 ml of YPG_{2%} medium containing 5 mM ALA in a 125 ml shake flask grown at 28° C., 150 rpm for 48 hrs. Genomic DNA was isolated from the cultures using the same method described in Example 8.

For Southern analysis 2 μg of genomic DNA was digested with 33 units of Nco I in a 50 μl reaction volume and subjected to 1% agarose electrophoresis in TAE buffer. The DNA in the gel was depurinated, denatured, and neutralized, and then transferred to a NYTRAN® Supercharge membrane as described in Example 8. The DNA was UV crosslinked to the membrane using a UV STRATALINKER™ and prehybridized for 1 hour at 42° C. in 20 ml of DIG Easy Hyb.

A probe to the 3′ flank of the hemA gene was generated using a PCR Dig Probe Synthesis Kit according to the manufacturer's instructions with the forward and reverse primers indicated below.

Forward (#065764)
(SEQ ID NO: 115)
5′-GACGCATACAATACAAGCATATGCTGTTGGTGTCT-3′
Reverse (#065765)
(SEQ ID NO: 116)
5′-AAGGCGTCTGGAAACAGAAGCTGCT-3′

The amplification reaction was composed of 1× HERCULASE® Reaction Buffer, 400 nM each primer, 200 μM DIG-labeled dUTP-containing dNTPs, 125 ng of T. reesei RutC30 genomic DNA, and 1.5 units HERCULASE® DNA polymerase. The reaction was incubated in an EPPENDORF® MASTERCYCLER® programmed for 1 cycle at 95° C. for 2 minutes; 25 cycles each at 95° C. for 30 seconds, 58° C. for 30 seconds, and 72° C. for 45 seconds; and 1 cycle at 72° C. for 7 minutes.

The probe was purified by 1% agarose gel electrophoresis in TAE buffer and the band corresponding to the probe was excised and agarose-extracted using a MINELUTE® Gel Extraction Kit. The probe was boiled for 5 minutes and added to 10 ml of DIG Easy Hyb to produce the hybridization solution. Hybridization was performed at 42° C. for 15-17 hours. The membrane was then washed under high stringency conditions in 2×SSC plus 0.1% SDS for 5 minutes at room temperature followed by two washes in 0.1×SSC plus 0.1% SDS for 15 minutes each at 65° C. The probe-target hybrids were detected by chemiluminescent assay (Roche Diagnostics, Indianapolis, Ind., USA) according to the manufacturer's instructions.

Southern analysis of the three transformants indicated that all three ALA aluxotrophic transformants contained the deletion cassette in a single copy at the hemA locus. One transformant JfyS2010-52-65 was used to cure out the hpt and tk markers. A fresh plate of spores was generated by transferring a plug of a 7 day old culture to a new PDA plate containing 5 mM ALA plate and incubating for 7 days at 28° C. Spores were collected in 10 ml 0.01% TWEEN® 20 using a sterile spreader. The concentration of spores was determined using a hemacytometer and 10⁶ spores were plated to 150 mm plates containing TrMM-G medium containing 1 mM ALA and 1 μM FdU.

Sixteen FdU-resistant spore isolates were obtained and DNA was extracted from 10 of those spore isolates as described above. The isolates were analyzed by Southern analysis as described above and the results indicated that all 10 of the spore isolates had excised the hpt/tk region between the repeats of the deletion cassette. One Fusarium venenatum strain JfyS2010-52-65-02 (ΔhemA, hpt-, tk-) was picked and archived.

The present invention is further described by the following numbered paragraphs:

[1] A method for deleting a gene or a portion thereof in the genome of a filamentous fungal cell, comprising:

(a) introducing into the filamentous fungal cell a nucleic acid construct comprising:
(i) a first polynucleotide comprising a dominant positively selectable marker coding sequence, which when expressed confers a dominant positively selectable phenotype on the filamentous fungal cell;
(ii) a second polynucleotide comprising a negatively selectable marker coding sequence, which when expressed confers a negatively selectable phenotype on the filamentous fungal cell;
(iii) a first repeat sequence located 5′ of the first and second polynucleotides and a second repeat sequence located 3′ of the first and second polynucleotides, wherein the first and second repeat sequences comprise identical sequences; and
(iv) a first flanking sequence located 5′ of components (i), (ii), and (iii) and a second flanking sequence located 3′ of the components (i), (ii), and (iii), wherein the first flanking sequence is identical to a first region of the genome of the filamentous fungal cell and the second flanking sequence is identical to a second region of the genome of the filamentous fungal cell, wherein (1) the first region is located 5′ of the gene or a portion thereof and the second region is located 3′ of the gene or a portion thereof of the filamentous fungal cell, (2) both of the first and second regions are located within the gene of the filamentous fungal cell, or (3) one of the first and second regions is located within the gene and the other of the first and second regions is located 5′ or 3′ of the gene of the filamentous fungal cell;

wherein the first and second flanking sequences undergo intermolecular homologous recombination with the first and second regions of the filamentous fungal cell, respectively, to delete and replace the gene or a portion thereof with the nucleic acid construct;

(b) selecting and isolating cells having a dominant positively selectable phenotype from step (a) by applying positive selection; and

(c) selecting and isolating a cell having a negatively selectable phenotype from the selected cells having the dominant positively selectable phenotype of step (b) by applying negative selection to force the first and second repeat sequences to undergo intramolecular homologous recombination to delete the first and second polynucleotides.

[2] The method of paragraph 1, wherein the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble), an acetamidase gene (amdS), a pyrithiamine resistance gene (ptrA), a puromycin-N-acetyl-transferase gene (pac), a neomycin-kanamycin phosphotransferase gene (neo), an acetyl CoA synthase gene (acuA), a D-serine dehydratase gene (dsdA), an ATP sulphurylase gene (sC), a mitochondrial ATP synthase subunit 9 gene (oliC), an aminoglycoside phosphotransferase 3′(I) (aph(3′)I) gene, and an aminoglycoside phosphotransferase 3′(II) (aph(3″)II gene.

[3] The method of paragraph 1, wherein the negatively selectable marker is encoded by a coding sequence of a gene selected from the group consisting of a thymidine kinase gene (tk), a orotidine-5′-phosphate decarboxylase gene (pyrG), and a cytosine deaminase gene (codA).

[4] The method of paragraph 1, wherein the dominant positively selectable marker is encoded by a coding sequence of a hygromycin phosphotransferase gene (hpt).

[5] The method of paragraph 4, wherein the hpt coding sequence is obtained from an E. coli hygromycin phosphotransferase gene.

[6] The method of paragraph 1, wherein the negatively selectable marker is encoded by a coding sequence of a thymidine kinase gene (tk).

[7] The method of paragraph 6, wherein the tk coding sequence is obtained from a Herpes simplex virus type 1 gene.

[8] The method of paragraph 1, wherein the dominant positively selectable marker is encoded by a coding sequence of a hygromycin phosphotransferase gene (hpt) and the negatively selectable marker is encoded by a coding sequence of a thymidine kinase gene (tk).

[9] The method of any of paragraphs 1-8, wherein the filamentous fungal cell is selected from the group consisting of an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

[10] The method of any of paragraphs 1-8, wherein the filamentous fungal cell is a pyrG auxotroph.

[11] The method of any of paragraphs 1-10, further comprising (d) introducing a polynucleotide encoding a polypeptide of interest into the isolated cell of step (c).

[12] The method of any of paragraphs 1-11, wherein the nucleic acid construct is contained in a linearized recombinant vector.

[13] The method of any of paragraphs 1-12, wherein the first region is located 5′ of the gene or a portion thereof and the second region is located 3′ of the gene or a portion thereof of the filamentous fungal cell.

[14] The method of any of paragraphs 1-12, wherein both of the first and second regions are located within the gene of the filamentous fungal cell.

[15] The method of any of paragraphs 1-12, one of the first and second regions is located within the gene and the other of the first and second regions is located 5′ or 3′ of the gene of the filamentous fungal cell.

[16] The method of any of paragraphs 1-15, wherein the first and second repeat sequences are identical to either the first flanking sequence or the second flanking sequence.

[17] The method of paragraph 1, wherein the entire gene is completely deleted leaving no foreign DNA.

[18] A nucleic acid construct for deleting a gene or a portion thereof in the genome of a filamentous fungal cell, comprising:
(i) a first polynucleotide comprising a dominant positively selectable marker coding sequence, which when expressed confers a dominant positively selectable phenotype on the filamentous fungal cell;
(ii) a second polynucleotide comprising a negatively selectable marker coding sequence, which when expressed confers a negatively selectable phenotype on the filamentous fungal cell;
(iii) a first repeat sequence located 5′ of the first and second polynucleotides and a second repeat sequence located 3′ of the first and second polynucleotides, wherein the first and second repeat sequences comprise identical sequences; and
(iv) a first flanking sequence located 5′ of components (i), (ii), and (iii) and a second flanking sequence located 3′ of the components (i), (ii), and (iii), wherein the first flanking sequence is identical to a first region of the genome of the filamentous fungal cell and the second flanking sequence is identical to a second region of the genome of the filamentous fungal cell, wherein (1) the first region is located 5′ of the gene or a portion thereof and the second region is located 3′ of the gene or a portion thereof of the filamentous fungal cell, (2) both of the first and second regions are located within the gene of the filamentous fungal cell, or (3) one of the first and second regions is located within the gene and the other of the first and second regions is located 5′ or 3′ of the gene of the filamentous fungal cell;

wherein the first and second flanking sequences undergo intermolecular homologous recombination with the first and second regions of the filamentous fungal cell, respectively, to delete and replace the gene or a portion thereof with the nucleic acid construct; and the first and second repeat sequences undergo intramolecular homologous recombination to delete the first and second polynucleotides.

[19] The nucleic acid construct of paragraph 18, wherein the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble), an acetamidase gene (amdS), a pyrithiamine resistance gene (ptrA), a puromycin-N-acetyl-transferase gene (pac), a neomycin-kanamycin phosphotransferase gene (neo), an acetyl CoA synthase gene (acuA), a D-serine dehydratase gene (dsdA), an ATP sulphurylase gene (sC), a mitochondrial ATP synthase subunit 9 gene (oliC), an aminoglycoside phosphotransferase 3′(I) (aph(3′)I) gene, and an aminoglycoside phosphotransferase 3′(II) (aph(3″)II) gene.

[20] The nucleic acid construct of paragraph 18, wherein the negatively selectable marker is encoded by a coding sequence of a gene selected from the group consisting of a thymidine kinase gene (tk), a orotidine-5′-phosphate decarboxylase gene (pyrG), and a cytosine deaminase gene (codA).

[21] The nucleic acid construct of paragraph 18, wherein the dominant positively selectable marker is encoded by a coding sequence of a hygromycin phosphotransferase gene (hpt).

[22] The nucleic acid construct of paragraph 21, wherein the hpt coding sequence is obtained from an E. coli hygromycin phosphotransferase gene.

[23] The nucleic acid construct of paragraph 18, wherein the negatively selectable marker is encoded by a coding sequence of a thymidine kinase gene (tk).

[24] The nucleic acid construct of paragraph 23, wherein the tk coding sequence is obtained from a Herpes simplex virus type 1 gene.

[25] The nucleic acid construct of paragraph 18, wherein the dominant positively selectable marker is encoded by a coding sequence of a hygromycin phosphotransferase gene (hpt) and the negatively selectable marker is encoded by a coding sequence of a thymidine kinase gene (tk).

[26] The nucleic acid construct of any of paragraphs 18-25, wherein the first region is located 5′ of the gene or a portion thereof and the second region is located 3′ of the gene or a portion thereof of the filamentous fungal cell.

[27] The nucleic acid construct of any of paragraphs 18-25, wherein both of the first and second regions are located within the gene of the filamentous fungal cell.

[28] The nucleic acid construct of any of paragraphs 18-25, wherein one of the first and second regions is located within the gene and the other of the first and second regions is located 5′ or 3′ of the gene of the filamentous fungal cell.

[29] The nucleic acid construct of any of paragraphs 18-28, wherein the first and second repeat sequences are identical to either the first flanking sequence or the second flanking sequence.

[30] A recombinant vector comprising the nucleic acid construct of any of paragraphs 18-29.

[31] A recombinant filamentous fungal cell comprising the nucleic acid construct of any of paragraphs 18-29.

[32] A method for introducing a polynucleotide into the genome of a filamentous fungal cell, comprising:

(a) introducing into the filamentous fungal cell a nucleic acid construct comprising:
(i) a first polynucleotide of interest;
(ii) a second polynucleotide comprising a dominant positively selectable marker coding sequence, which when expressed confers a dominant positively selectable phenotype on the filamentous fungal cell;
(iii) a third polynucleotide comprising a negatively selectable marker coding sequence, which when expressed confers a negatively selectable phenotype on the filamentous fungal cell;
(iv) a first repeat sequence located 5′ of the second and third polynucleotides and a second repeat sequence located 3′ of the second and third polynucleotides, wherein the first and second repeat sequences comprise identical sequences and the first polynucleotide of interest is located either 5′ of the first repeat or 3′ of the second repeat; and
(v) a first flanking sequence located 5′ of components (i), (ii), (iii), and (iv) and a second flanking sequence located 3′ of the components (i), (ii), (iii), and (iv), wherein the first flanking sequence is identical to a first region of the genome of the filamentous fungal cell and the second flanking sequence is identical to a second region of the genome of the filamentous fungal cell;

wherein the first and second flanking sequences undergo intermolecular homologous recombination with the first and second regions of the genome of the filamentous fungal cell, respectively, to introduce the nucleic acid construct into the genome of the filamentous fungal cell;

(b) selecting cells having a dominant positively selectable phenotype from step (a) by applying positive selection; and

(c) selecting and isolating a cell having a negatively selectable phenotype from the selected cells having the dominant positively selectable phenotype of step (b) by applying negative selection to force the first and second repeat sequences to undergo intramolecular homologous recombination to delete the second and third polynucleotides.

[33] The method of paragraph 32, wherein the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble), an acetamidase gene (amdS), a pyrithiamine resistance gene (ptrA), a puromycin-N-acetyl-transferase gene (pac), a neomycin-kanamycin phosphotransferase gene (neo), an acetyl CoA synthase gene (acuA), a D-serine dehydratase gene (dsdA), an ATP sulphurylase gene (sC), a mitochondrial ATP synthase subunit 9 gene (oliC), an aminoglycoside phosphotransferase 3′(I) (aph(3′)I) gene, and an aminoglycoside phosphotransferase 3′(II) (aph(3′)II) gene.

[34] The method of paragraph 32, wherein the negatively selectable marker is encoded by a coding sequence of a gene selected from the group consisting of a thymidine kinase gene (tk), a orotidine-5′-phosphate decarboxylase gene (pyrG), and a cytosine deaminase gene (codA).

[35] The method of paragraph 32, wherein the dominant positively selectable marker is encoded by a coding sequence of a hygromycin phosphotransferase gene (hpt).

[36] The method of paragraph 35, wherein the hpt coding sequence is obtained from an E. coli hygromycin phosphotransferase gene.

[32] The method of paragraph 32, wherein the negatively selectable marker is encoded by a coding sequence of a thymidine kinase gene (tk).

[38] The method of paragraph 37, wherein the tk coding sequence is obtained from a Herpes simplex virus type 1 gene.

[39] The method of paragraph 32, wherein the dominant positively selectable marker is encoded by a coding sequence of a hygromycin phosphotransferase gene (hpt) and the negatively selectable marker is encoded by a coding sequence of a thymidine kinase gene (tk).

[40] The method of any of paragraphs 32-39, wherein the filamentous fungal cell is selected from the group consisting of an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

[41] The method of any of paragraphs 32-39, wherein the filamentous fungal cell is a pyrG auxotroph.

[42] The method of any of paragraphs 32-41, wherein the nucleic acid construct is contained in a linearized recombinant vector.

[43] The method of any of paragraphs 32-42, wherein the first region is located 5′ of the gene or a portion thereof and the second region is located 3′ of the gene or a portion thereof of the filamentous fungal cell.

[44] The method of any of paragraphs 32-42, wherein both of the first and second regions are located within the gene of the filamentous fungal cell.

[45] The method of any of paragraphs 32-42, wherein one of the first and second regions is located within the gene and the other of the first and second regions is located 5′ or 3′ of the gene of the filamentous fungal cell.

[46] The method of any of paragraphs 32-45, wherein the first and second repeat sequences are identical to either the first flanking sequence or the second flanking sequence.

[47] A nucleic acid construct for introducing a polynucleotide into the genome of a filamentous fungal cell, comprising:
(i) a first polynucleotide of interest;
(ii) a second polynucleotide comprising a dominant positively selectable marker coding sequence, which when expressed confers a dominant positively selectable phenotype on the filamentous fungal cell;
(iii) a third polynucleotide comprising a negatively selectable marker coding sequence, which when expressed confers a negatively selectable phenotype on the filamentous fungal cell;
(iv) a first repeat sequence located 5′ of the first and second polynucleotides and a second repeat sequence located 3′ of the first and second polynucleotides, wherein the first and second repeat sequences comprise identical sequences and the first polynucleotide encoding the polypeptide of interest is located either 5′ of the first repeat or 3′ of the second repeat; and
(v) a first flanking sequence located 5′ of components (i), (ii), (iii), and (iv) and a second flanking sequence located 3′ of the components (i), (ii), (iii), and (iv), wherein the first flanking sequence is identical to a first region of the genome of the filamentous fungal cell and the second flanking sequence is identical to a second region of the genome of the filamentous fungal cell;

wherein the first and second flanking sequences undergo intermolecular homologous recombination with the first and second regions of the genome of the filamentous fungal cell, respectively, to introduce the nucleic acid construct into the genome of the filamentous fungal cell; and the first and second repeat sequences can undergo intramolecular homologous recombination to delete the second and third polynucleotides.

[48] The nucleic acid construct of paragraph 47, wherein the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble), an acetamidase gene (amdS), a pyrithiamine resistance gene (ptrA), a puromycin-N-acetyl-transferase gene (pac), a neomycin-kanamycin phosphotransferase gene (neo), an acetyl CoA synthase gene (acuA), a D-serine dehydratase gene (dsdA), an ATP sulphurylase gene (sC), a mitochondrial ATP synthase subunit 9 gene (oliC), an aminoglycoside phosphotransferase 3′(I) (aph(3′)I) gene, and an aminoglycoside phosphotransferase 3′(II) aph (3′)II gene.

[49] The nucleic acid construct of paragraph 47, wherein the negatively selectable marker is encoded by a coding sequence of a gene selected from the group consisting of a thymidine kinase gene (tk), a orotidine-5′-phosphate decarboxylase gene (pyrG), and a cytosine deaminase gene (codA).

[50] The nucleic acid construct of paragraph 47, wherein the dominant positively selectable marker is encoded by a coding sequence of a hygromycin phosphotransferase gene (hpt).

[51] The nucleic acid construct of paragraph 47, wherein the negatively selectable marker is encoded by a coding sequence of a thymidine kinase gene (tk).

[52] The nucleic acid construct of paragraph 47, wherein the dominant positively selectable marker is encoded by a coding sequence of a hygromycin phosphotransferase gene (hpt) and the negatively selectable marker is encoded by a coding sequence of a thymidine kinase gene (tk).

[53] The nucleic acid construct of paragraph 47, wherein the hpt coding sequence is obtained from an E. coli hygromycin phosphotransferase gene.

[54] The nucleic acid construct of paragraph 47, wherein the tk coding sequence is obtained from a Herpes simplex virus type 1 gene.

[55] The nucleic acid construct of any of paragraphs 47-54, wherein the first region is located 5′ of the gene or a portion thereof and the second region is located 3′ of the gene or a portion thereof of the filamentous fungal cell.

[56] The nucleic acid construct of any of paragraphs 47-54, wherein both of the first and second regions are located within the gene of the filamentous fungal cell.

[57] The nucleic acid construct of any of paragraphs 47-54, wherein one of the first and second regions is located within the gene and the other of the first and second regions is located 5′ or 3′ of the gene of the filamentous fungal cell

[58] The nucleic acid construct of any of paragraphs 47-57, wherein the first and second repeat sequences are identical to either the first flanking sequence or the second flanking sequence.

[59] A recombinant vector comprising the nucleic acid construct of any of paragraphs 47-58.

[60] A recombinant filamentous fungal cell comprising the nucleic acid construct of any of paragraphs 47-58.

[61] A method of producing a polypeptide, comprising (a) cultivating a filamentous fungal cell, obtained according to any of paragraphs 1-17, under conditions conducive for production of a polypeptide; and (b) recovering the polypeptide.

[62] The method of paragraph 61, wherein the polypeptide is native to the filamentous fungal cell.

[63] The method of paragraph 61, wherein the polypeptide is a foreign (heterologous) polypeptide encoded by a polynucleotide, which has been introduced into the filamentous fungal cell.

[64] A method of producing a polypeptide, comprising (a) cultivating a filamentous fungal cell, obtained according to any of paragraphs 32-46, under conditions conducive for production of a polypeptide; and (b) recovering the polypeptide.

[65] The method of paragraph 65, wherein the polypeptide is native to the filamentous fungal cell.

[66] The method of paragraph 65, wherein the polypeptide is a foreign (heterologous) polypeptide encoded by a polynucleotide, which has been introduced into the filamentous fungal cell.

[67] An isolated orotidine-5′-phosphate decarboxylase selected from the group consisting of: (a) an orotidine-5′-phosphate decarboxylase comprising an amino acid sequence having preferably at least 70%, more preferably at least 75%, more preferably at least 80%, more preferably at least 85%, even more preferably at least 90%, even more preferably at least 95% identity, and most preferably at least 95%, at least 97%, at least 98%, or at least 99% identity to the mature polypeptide of SEQ ID NO: 52; (b) an orotidine-5′-phosphate decarboxylase encoded by a polynucleotide that hybridizes under preferably at least medium stringency conditions, more preferably at least medium stringency conditions, even more preferably at least high stringency conditions, and most preferably very high stringency conditions with the mature polypeptide coding sequence of SEQ ID NO: 51 or its full-length complementary strand; and (c) an orotidine-5′-phosphate decarboxylase encoded by a polynucleotide comprising a nucleotide sequence having preferably at least 80%, more preferably at least 85%, even more preferably at least 90%, even more preferably at least 95% identity, and most preferably, at least 96%, at least 97%, at least 98%, or at least 99% identity to the mature polypeptide coding sequence of SEQ ID NO: 51.

[68] The isolated orotidine-5′-phosphate decarboxylase of paragraph 67 comprising or consisting of SEQ ID NO: 52, or a fragment thereof having orotidine-5′-phosphate decarboxylase activity.

[69] An isolated polynucleotide encoding the orotidine-5′-phosphate decarboxylase of paragraph 67 or 68.

[70] The isolated polynucleotide of paragraph 69, comprising or consisting of SEQ ID NO: 51 or a subsequence thereof that encodes a fragment having orotidine-5′-phosphate decarboxylase activity.

[71] A nucleic acid construct comprising the polynucleotide of paragraph 69 or 70.

[72] A recombinant expression vector comprising the polynucleotide of paragraph 69 or 70.

[73] A recombinant filamentous fungal cell comprising the polynucleotide of paragraph 69 or 70.

[74] A method of producing the orotidine-5′-phosphate decarboxylase of paragraph 67 or 68, comprising: cultivating a host cell comprising a nucleic acid construct comprising a nucleotide sequence encoding the orotidine-5′-phosphate decarboxylase under conditions conducive for production of the polypeptide.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A method for deleting a gene or a portion thereof in the genome of a filamentous fungal cell, comprising:
(a) introducing into the filamentous fungal cell a nucleic acid construct comprising:
(i) a first polynucleotide comprising a dominant positively selectable marker coding sequence, which when expressed confers a dominant positively selectable phenotype on the filamentous fungal cell;
(ii) a second polynucleotide comprising a negatively selectable marker coding sequence, which when expressed confers a negatively selectable phenotype on the filamentous fungal cell;
(iii) a first repeat sequence located 5′ of the first and second polynucleotides and a second repeat sequence located 3′ of the first and second polynucleotides, wherein the first and second repeat sequences comprise identical sequences; and
(iv) a first flanking sequence located 5′ of components (i), (ii), and (iii) and a second flanking sequence located 3′ of the components (i), (ii), and (iii), wherein the first flanking sequence is identical to a first region of the genome of the filamentous fungal cell and the second flanking sequence is identical to a second region of the genome of the filamentous fungal cell, wherein (1) the first region is located 5′ of the gene or a portion thereof and the second region is located 3′ of the gene or a portion thereof of the filamentous fungal cell, (2) both of the first and second regions are located within the gene of the filamentous fungal cell, or (3) one of the first and second regions is located within the gene and the other of the first and second regions is located 5′ or 3′ of the gene of the filamentous fungal cell;
wherein the first and second flanking sequences undergo intermolecular homologous recombination with the first and second regions of the filamentous fungal cell, respectively, to delete and replace the gene or a portion thereof with the nucleic acid construct;
(b) selecting and isolating cells having a dominant positively selectable phenotype from step (a) by applying positive selection; and
(c) selecting and isolating a cell having a negatively selectable phenotype from the selected cells having the dominant positively selectable phenotype of step (b) by applying negative selection to force the first and second repeat sequences to undergo intramolecular homologous recombination to delete the first and second polynucleotides.

2. The method of claim 1, wherein the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble), an acetamidase gene (amdS), a pyrithiamine resistance gene (ptrA), a puromycin-N-acetyl-transferase gene (pac), a neomycin-kanamycin phosphotransferase gene (neo), an acetyl CoA synthase gene (acuA), a D-serine dehydratase gene (dsdA), an ATP sulphurylase gene (sC), a mitochondrial ATP synthase subunit 9 gene (oliC), an aminoglycoside phosphotransferase 3′(I) (aph(3′)I) gene, and an aminoglycoside phosphotransferase 3′(II) (aph(3′)II gene.

3. The method of claim 1, wherein the negatively selectable marker is encoded by a coding sequence of a gene selected from the group consisting of a thymidine kinase gene (tk), a orotidine-5′-phosphate decarboxylase gene (pyrG), and a cytosine deaminase gene (codA).

4. The method of claim 1, further comprising (d) introducing a polynucleotide encoding a polypeptide of interest into the isolated cell of step (c).

5. (canceled)

6. (canceled)

7. A nucleic acid construct for deleting a gene or a portion thereof in the genome of a filamentous fungal cell, comprising:
(i) a first polynucleotide comprising a dominant positively selectable marker coding sequence, which when expressed confers a dominant positively selectable phenotype on the filamentous fungal cell;
(ii) a second polynucleotide comprising a negatively selectable marker coding sequence, which when expressed confers a negatively selectable phenotype on the filamentous fungal cell;
(iii) a first repeat sequence located 5′ of the first and second polynucleotides and a second repeat sequence located 3′ of the first and second polynucleotides, wherein the first and second repeat sequences comprise identical sequences; and
(iv) a first flanking sequence located 5′ of components (i), (ii), and (iii) and a second flanking sequence located 3′ of the components (i), (ii), and (iii), wherein the first flanking sequence is identical to a first region of the genome of a filamentous fungal cell and the second flanking sequence is identical to a second region of the genome of the filamentous fungal cell, wherein (1) the first region is located 5′ of the gene or a portion thereof and the second region is located 3′ of the gene or a portion thereof of the filamentous fungal cell, (2) both of the first and second regions are located within the gene of the filamentous fungal cell, or (3) one of the first and second regions is located within the gene and the other of the first and second regions is located 5′ or 3′ of the gene of the filamentous fungal cell;
wherein the first and second flanking sequences undergo intermolecular homologous recombination with the first and second regions of the filamentous fungal cell, respectively, to delete and replace the gene or a portion thereof with the nucleic acid construct; and the first and second repeat sequences undergo intramolecular homologous recombination to delete the first and second polynucleotides.

8. The nucleic acid construct of claim 7, wherein the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble), an acetamidase gene (amdS), a pyrithiamine resistance gene (ptrA), a puromycin-N-acetyl-transferase gene (pac), a neomycin-kanamycin phosphotransferase gene (neo), an acetyl CoA synthase gene (acuA), a D-serine dehydratase gene (dsdA), an ATP sulphurylase gene (sC), a mitochondrial ATP synthase subunit 9 gene (oliC), an aminoglycoside phosphotransferase 3′(I) (aph(3′)I) gene, and an aminoglycoside phosphotransferase 3′(II) (aph(3′)II) gene.

9. The nucleic acid construct of claim 7, wherein the negatively selectable marker is encoded by a coding sequence of a gene selected from the group consisting of a thymidine kinase gene (tk), a orotidine-5′-phosphate decarboxylase gene (pyrG), and a cytosine deaminase gene (codA).

10. (canceled)

11. A recombinant filamentous fungal cell comprising the nucleic acid construct of claim 7.

12. A method for introducing a polynucleotide into the genome of a filamentous fungal cell, comprising:
(a) introducing into the filamentous fungal cell a nucleic acid construct comprising:
(i) a first polynucleotide of interest;
(ii) a second polynucleotide comprising a dominant positively selectable marker coding sequence, which when expressed confers a dominant positively selectable phenotype on the filamentous fungal cell;
(iii) a third polynucleotide comprising a negatively selectable marker coding sequence, which when expressed confers a negatively selectable phenotype on the filamentous fungal cell;
(iv) a first repeat sequence located 5′ of the second and third polynucleotides and a second repeat sequence located 3′ of the second and third polynucleotides, wherein the first and second repeat sequences comprise identical sequences and the first polynucleotide of interest is located either 5′ of the first repeat or 3′ of the second repeat; and
(v) a first flanking sequence located 5′ of components (i), (ii), (iii), and (iv) and a second flanking sequence located 3′ of the components (i), (ii), (iii), and (iv), wherein the first flanking sequence is identical to a first region of the genome of the filamentous fungal cell and the second flanking sequence is identical to a second region of the genome of the filamentous fungal cell;
wherein the first and second flanking sequences undergo intermolecular homologous recombination with the first and second regions of the genome of the filamentous fungal cell, respectively, to introduce the nucleic acid construct into the genome of the filamentous fungal cell;
(b) selecting cells having a dominant positively selectable phenotype from step (a) by applying positive selection; and
(c) selecting and isolating a cell having a negatively selectable phenotype from the selected cells having the dominant positively selectable phenotype of step (b) by applying negative selection to force the first and second repeat sequences to undergo intramolecular homologous recombination to delete the second and third polynucleotides.

13. The method of claim 12, wherein the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble), an acetamidase gene (amdS), a pyrithiamine resistance gene (ptrA), a puromycin-N-acetyl-transferase gene (pac), a neomycin-kanamycin phosphotransferase gene (neo), an acetyl CoA synthase gene (acuA), a D-serine dehydratase gene (dsdA), an ATP sulphurylase gene (sC), a mitochondrial ATP synthase subunit 9 gene (oliC), an aminoglycoside phosphotransferase 3′(I) (aph(3′)I) gene, and an aminoglycoside phosphotransferase 3′(II) (aph(3′)II) gene.

14. The method of claim 12, wherein the negatively selectable marker is encoded by a coding sequence of a gene selected from the group consisting of a thymidine kinase gene (tk), a orotidine-5′-phosphate decarboxylase gene (pyrG), and a cytosine deaminase gene (codA).

15. (canceled)

16. A nucleic acid construct for introducing a polynucleotide into the genome of a filamentous fungal cell, comprising:
(i) a first polynucleotide of interest;
(ii) a second polynucleotide comprising a dominant positively selectable marker coding sequence, which when expressed confers a dominant positively selectable phenotype on the filamentous fungal cell;
(iii) a third polynucleotide comprising a negatively selectable marker coding sequence, which when expressed confers a negatively selectable phenotype on the filamentous fungal cell;
(iv) a first repeat sequence located 5′ of the first and second polynucleotides and a second repeat sequence located 3′ of the first and second polynucleotides, wherein the first and second repeat sequences comprise identical sequences and the first polynucleotide encoding the polypeptide of interest is located either 5′ of the first repeat or 3′ of the second repeat; and
(v) a first flanking sequence located 5′ of components (i), (ii), (iii), and (iv) and a second flanking sequence located 3′ of the components (i), (ii), (iii), and (iv), wherein the first flanking sequence is identical to a first region of the genome of the filamentous fungal cell and the second flanking sequence is identical to a second region of the genome of the filamentous fungal cell;
wherein the first and second flanking sequences undergo intermolecular homologous recombination with the first and second regions of the genome of the filamentous fungal cell, respectively, to introduce the nucleic acid construct into the genome of the filamentous fungal cell; and the first and second repeat sequences can undergo intramolecular homologous recombination to delete the second and third polynucleotides.

17. The nucleic acid construct of claim 16, wherein the dominant positively selectable marker is encoded by a coding sequence of a gene selected from the group consisting of a hygromycin phosphotransferase gene (hpt), a phosphinothricin acetyltransferase gene (pat), a bleomycin, zeocin and phleomycin resistance gene (ble), an acetamidase gene (amdS), a pyrithiamine resistance gene (ptrA), a puromycin-N-acetyl-transferase gene (pac), a neomycin-kanamycin phosphotransferase gene (neo), an acetyl CoA synthase gene (acuA), a D-serine dehydratase gene (dsdA), an ATP sulphurylase gene (sC), a mitochondrial ATP synthase subunit 9 gene (oliC), an aminoglycoside phosphotransferase 3′(I) (aph (3′)I) gene, and an aminoglycoside phosphotransferase 3′(II) aph (3′)II gene.

18. The nucleic acid construct of claim 16, wherein the negatively selectable marker is encoded by a coding sequence of a gene selected from the group consisting of a thymidine kinase gene (tk), a orotidine-5′-phosphate decarboxylase gene (pyrG), and a cytosine deaminase gene (codA).

19. (canceled)

20. A recombinant filamentous fungal cell comprising the nucleic acid construct of claim 16.

21. A method of producing a polypeptide, comprising (a) cultivating a filamentous fungal cell, obtained according to claim 1, under conditions conducive for production of a polypeptide; and (b) recovering the polypeptide.

22. A method of producing a polypeptide, comprising (a) cultivating a filamentous fungal cell, obtained according to claim 12, under conditions conducive for production of a polypeptide; and (b) recovering the polypeptide.

23. An isolated orotidine-5′-phosphate decarboxylase selected from the group consisting of: (a) an orotidine-5′-phosphate decarboxylase comprising an amino acid sequence having preferably at least 70%, more preferably at least 75%, more preferably at least 80%, more preferably at least 85%, even more preferably at least 90%, even more preferably at least 95% identity, and most preferably at least 95%, at least 97%, at least 98%, or at least 99% identity to the mature polypeptide of SEQ ID NO: 52; (b) an orotidine-5′-phosphate decarboxylase encoded by a polynucleotide that hybridizes under preferably at least medium stringency conditions, more preferably at least medium stringency conditions, even more preferably at least high stringency conditions, and most preferably very high stringency conditions with the mature polypeptide coding sequence of SEQ ID NO: 51 or its full-length complementary strand; and (c) an orotidine-5′-phosphate decarboxylase encoded by a polynucleotide comprising a nucleotide sequence having preferably at least 80%, more preferably at least 85%, even more preferably at least 90%, even more preferably at least 95% identity, and most preferably, at least 96%, at least 97%, at least 98%, or at least 99% identity to the mature polypeptide coding sequence of SEQ ID NO: 51.

24. (canceled)

25. An isolated polynucleotide encoding the orotidine-5′-phosphate decarboxylase of claim 23.

26. A method of producing the orotidine-5′-phosphate decarboxylase of claim 23, comprising: cultivating a host cell comprising a nucleic acid construct comprising a nucleotide sequence encoding the orotidine-5′-phosphate decarboxylase under conditions conducive for production of the polypeptide.


SEQUENCE LISTING (116 sequences)

SEQ ID NO: 1 - DNA, 60 bp, Aspergillus nidulans
gacgaattctctagaagatctctcgaggagctcaagcttctgtacagtgaccggtgactc

SEQ ID NO: 2 - DNA, 27 bp, Aspergillus nidulans
gacgaattccgatgaatgtgtgtcctg

SEQ ID NO: 3 - DNA, 20 bp, Herpes simplex virus
cgagtgtaaactgggagttg

SEQ ID NO: 4 - DNA, 20 bp, Herpes simplex virus
gagcaagcccagatgagaac

SEQ ID NO: 5 - DNA, 20 bp, Herpes simplex virus
ggcgattggtcgtaatccag

SEQ ID NO: 6 - DNA, 20 bp, Herpes simplex virus
tcttcgaccgccatcccatc

SEQ ID NO: 7 - DNA, 1026 bp, Escherichia coli
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 8 - protein, 343 aa, Escherichia coli
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRARNSK

SEQ ID NO: 9 - DNA, 24 bp, Escherichia coli
gggttcgaattcatttaaacggct

SEQ ID NO: 10 - DNA, 24 bp, Escherichia coli
gggagcgctcaatattcatctctc

SEQ ID NO: 11 - DNA, 31 bp, Escherichia coli
gggtaccccaagggcgtattctgcagatggg

SEQ ID NO: 12 - DNA, 31 bp, Escherichia coli
cccatctgcagaatacgcccttggggtaccc

SEQ ID NO: 13 - DNA, 27 bp, Escherichia coli
ggggtaccttcatttaaacggcttcac

SEQ ID NO: 14 - DNA, 25 bp, Escherichia coli
ggggtacccgaccagcagacggccc

SEQ ID NO: 15 - DNA, 28 bp, Aspergillus oryzae
tcccccgggtctctggtactcttcgatc

SEQ ID NO: 16 - DNA, 25 bp, Aspergillus oryzae
ggggtacccgaccagcagacggccc

SEQ ID NO: 17 - DNA, 27 bp, Aspergillus oryzae
ggggtacctctctggtactcttcgatc

SEQ ID NO: 18 - DNA, 26 bp, Aspergillus oryzae
tcccccgggcgaccagcagacggccc

SEQ ID NO: 19 - DNA, 31 bp, Escherichia coli; identical to SEQ ID NO: 11

SEQ ID NO: 20 - DNA, 31 bp, Escherichia coli; identical to SEQ ID NO: 12

SEQ ID NO: 21 - DNA, 1193 bp, Neurospora crassa
atgtcgacaagtaggaaacgcagccacactggtccctcaagcagtcgtttgctgagcgggtagagagctcgacgcatcccctcaccagctacctcttccgcctgatggaggtcaagcagtccaacctctgcctcagcgccgatgtcgagcacgcgcgggatctcctcgcccttgccgacaaggtgggcccctcgattgtcgtcctcaagacccactacgacctgatcacagggtgggactaccacccgcacacgggcaccggcgccaagctggccgcccttgcccggaagcacggcttcctcatcttcgaggaccgcaagttcgtcgacattggcagcaccgtccagaagcagtacacggccggcaccgcgcgcattgtcgaatgggcccacatcaccaacgccgacatccacgccggagaggccatggtgagcgccatggcccaggccgcgcaaaagtggagggagcgcatcccctacgaggtcaagacgtcggtttcggtgggcaccccggtcgcggaccagttcgccgacgaggaagccgaggaccaggttgaggagctgcgcaaggtcgtcacccgcgagaccagcaccaccacaaaggacacggatgggaggaagagtagcatcgtctccatcacgaccgtcacgcagacatatgagccggccgactcgccacgtctggtcaagaccatctcggaggacgatgagatggtgttccccggcatcgaggaggcgcctctggaccgcggcctgctgatcttggcccagatgtcgtccaagggctgcctcatggacggcaagtacacatgggagtgtgtcaaggcggcccgcaagaacaagggctttgtcatgggctacgttgcgcagcagaacctgaacggcattaccaaggaagctttggccccaagctacgaagacggcgaaagcacgacagaggaagaagcgcaagcagacaacttcatccacatgacacccggctgcaagttgccgccaccaggagaggaagcgcctcagggcgacggactgggtcagcagtacaacacgccggataaccttgtcaacatcaagggcaccgatatcgcgattgttgggcgtggcatcatcaccgcggcggatcctccggccgaggctgagcgctacaggaggaaagcctggaaggcgtaccaggatcgccgggagcgtctggcatag

SEQ ID NO: 22 - protein, 397 aa, Neurospora crassa
MSTSQETQPHWSLKQSFAERVESSTHPLTSYLFRLMEVKQSNLCLSADVEHARDLLALADKVGPSIVVLKTHYDLITGWDYHPHTGTGAKLAALARKHGFLIFEDRKFVDIGSTVQKQYTAGTARIVEWAHITNADIHAGEAMVSAMAQAAQKWRERIPYEVKTSVSVGTPVADQFADEEAEDQVEELRKVVTRETSTTTKDTDGRKSSIVSITTVTQTYEPADSPRLVKTISEDDEMVFPGIEEAPLDRGLLILAQMSSKGCLMDGKYTWECVKAARKNKGFVMGYVAQQNLNGITKEALAPSYEDGESTTEEEAQADNFIHMTPGCKLPPPGEEAPQGDGLGQQYNTPDNLVNIKGTDIAIVGRGIITAADPPAEAERYRRKAWKAYQDRRERLA

SEQ ID NO: 23 - DNA, 20 bp, Neurospora crassa
gtcaggaaacgcagccacac

SEQ ID NO: 24 - DNA, 20 bp, Neurospora crassa
aggcagcccttggacgacat

SEQ ID NO: 25 - DNA, 25 bp, Fusarium venenatum
gccatgcgatccagcgtttgaatcc

SEQ ID NO: 26 - DNA, 25 bp, Fusarium venenatum
gcgtccgcaactgacgatggtcctc

SEQ ID NO: 27 - DNA, 21 bp, Fusarium venenatum
cagataccacagacggcaagc

SEQ ID NO: 28 - DNA, 18 bp, Fusarium venenatum
gggcagttcggtttcagg

SEQ ID NO: 29 - DNA, 1203 bp, Fusarium venenatum
atggagaactttcccactgagtattttctcaacacttctgtgcgccttctcgagtacattcgataccgagatagcaattatacccgggaagagcgtatcgagaatttgcactatgcttacaacaaggctgctcatcactttgctcagccacgacaacagcagctgctcaaggtagaccctaagcgactacaggcttccctccaaactattgttggcatggtggtatacagttgggcaaaggtctccaaagagtgtatggcggatctatctattcattacacgtacacactcgttttggatgacagcagcgatgatccgtatccagccatgatgaactatttcaacgatcttcaggctggacgagaacaggcccacccatggtgggcgcttgttaatgagcactttcccaatgtccttcgacattttggtcccttctgctcattgaaccttatccgcagcactcttgactgtaagtaccctggctctattatttcaccgccttaataagctaacagtgatggaattatagtttttgagggatgctggatcgagcagtacaactttggaggatttccaggatctcatgactatcctcagtttcttcgacgcatgaatggcttgggtcactgtgtcggggcttctttgtggcccaaagagcagtttgatgagagaggtctattccttgaaatcacatcagccattgctcagatggagaactggatggtctgggtcaatgatctcatgtctttctacaaggagttcgatgatgagcgtgaccagatcagtctcgtcaagaactacgtcgtctctgatgagatcactctccacgaagctttagagaagctcacccaggacactctacactcgtccaagcagatggtagctgtcttctctgacaaggaccctcaggtgatggacacgattgagtgcttcatgcacggctatgtcacgtggcacttgtgcgatcacaggtaccgtctgaatgagatctacgaaaaggtcaaaggacaaaagaccgaggacgctcagaagttctgcaagttctatgagcaggctgctaacgtcggagccgtttcgccctcggagtgggcttatccacctattgcgcaactggcaaacattcggtccaaggatgtgaaggatgtgaaggatgtgaaggagattcagaagcctctgctgagctcaattgagctagtggaatga

SEQ ID NO: 30 - protein, 380 aa, Fusarium venenatum
MENFPTEYFLNTSVRLLEYIRYRDSNYTREERIENLHYAYNKAAHHFAQPRQQQLLKVDPKRLQASLQTIVGMVVYSWAKVSKECMADLSIHYTYTLVLDDSSDDPYPAMMNYFNDLQAGREQAHPWWALVNEHFPNVLRHFGPFCSLNLIRSTLDFFEGCWIEQYNFGGFPGSHDYPQFLRRMNGLGHCVGASLWPKEQFDERGLFLEITSAIAQMENWMVWVNDLMSFYKEFDDERDQISLVKNYVVSDEITLHEALEKLTQDTLHSSKQMVAVFSDKDPQVMDTIECFMHGYVTWHLCDHRYRLNEIYEKVKGQKTEDAQKFCKFYEQAANVGAVSPSEWAYPPIAQLANIRSKDVKDVKDVKEIQKPLLSSIELVE

SEQ ID NO: 31 - DNA, 23 bp, Fusarium venenatum
gggagatcttcgttatctgtgcc

SEQ ID NO: 32 - DNA, 29 bp, Fusarium venenatum
gggagatcttagtagtcggcatttgaaac

SEQ ID NO: 33 - DNA, 43 bp, Fusarium venenatum
caagtaacagacgcgacagcttgcaaaatcttcgttatctgtg

SEQ ID NO: 34 - DNA, 43 bp, Fusarium venenatum
cacagataacgaagattttgcaagctgtcgcgtctgttacttg

SEQ ID NO: 35 - DNA, 40 bp, Escherichia coli
ttgaactctcagatcccttcatttaaacggcttcacgggc

SEQ ID NO: 36 - DNA, 40 bp, Escherichia coli
cagataacgaagatctacgcccttggggtacccaatattc

SEQ ID NO: 37 - DNA, 1131 bp, Herpes simplex virus
atggcttcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcggggccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactga

SEQ ID NO: 38 - protein, 376 aa, Herpes simplex virus
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRGPELLAPNGDLYNVFAWALDVLAKRLRSMHVFILDYDQSPAGCRDALLQLTSGMVQTHVTTPGSIPTICDLARTFAREMGEAN

SEQ ID NO: 39 - DNA, 40 bp, Herpes simplex virus
gccgactactagatcgaccggtgactctttctggcatgcg

SEQ ID NO: 40 - DNA, 40 bp, Herpes simplex virus
cagataacgaagatctgagagttcaaggaagaaacagtgc

SEQ ID NO: 41 - DNA, 27 bp, Herpes simplex virus
ccctgtttcggggccccgagttgctgg

SEQ ID NO: 42 - DNA, 27 bp, Herpes simplex virus
ccagcaactcggggccccgaaacaggg

SEQ ID NO: 43 - DNA, 29 bp, Fusarium venenatum
gtgggaggatctgatggatcaccatgggc

SEQ ID NO: 44 - DNA, 31 bp, Fusarium venenatum
ccgggtttcgttccgaacgatctttacaagg

SEQ ID NO: 45 - DNA, 43 bp, Fusarium venenatum
gtttaaacggcgcgcccgacaaaacaaggctactgcaggcagg

SEQ ID NO: 46 - DNA, 32 bp, Fusarium venenatum
ttgtcgcccgggaatactccaactaggccttg

SEQ ID NO: 47 - DNA, 33 bp, Fusarium venenatum
agtattcccgggcgacaaaacaaggctactgca

SEQ ID NO: 48 - DNA, 36 bp, Fusarium venenatum
atttaaatcctgcaggaatactccaactaggccttg

SEQ ID NO: 49 - DNA, 35 bp, Escherichia coli
aaaacccgggccttcatttaaacggcttcacgggc

SEQ ID NO: 50 - DNA, 40 bp, Escherichia coli
aaaacccgggagatctacgcccttggggtacccaatattc

SEQ ID NO: 51 - DNA, 1098 bp, Fusarium venenatum
atgtcgtcgcatccgtccctcaaggcgactttcgccagtcgagctgagacagcctctcatcctcttagccgacatctctacaaacttatggacctcaaggcctcgaacctttgtctcagcgccgatgtcgcaaccgcccgcgagctcctctacttcgccgacaagatcggcccctctatcgtcgtcctcaagactcattatgacatggtggctggctgggatttcgacccccgaacaggaaccggtgccaagctcgcatcgctagcccgcaagcacggtttcctcatctttgaggatcgcaagtttggtgacattggcaacacggtcgagctgcagtacaccagtggtgctgcccgcattatcgagtgggcacacattgtcaatgtgaacatggtccctggaaaggcttctgttacgtctttggctcacgccgccaaccgatggctggagcgataccactatgaggtcaagacatctatcagcattggaacccctacggccagtcaactagacgaggacagcgagcgctcagatggcgagaaccaaaagagcgcacctgaacttggccgcgacaacggacgcaaaggcagcatcgtctctaccactaccgtcactcagcagtacgagtcggccgattcaccacgcctcgtcaagacgatccccgagggcgacgaaacagtattcgccggtatcgacgaggcacctatcgagagaggtctgcttatcctagcacaaatgtcaagtgaaggcaacttcatgaacaaggaatacacacaagcttgtgtagaggccgcgcgggaacacaagagctttgttatgggtttcatttcacaggagtgtctcaacacacaacctgacgatgatttcatccacatgacccctggctgccaattgcctcctgagggtgcggatgagaacgaggctatcaagggagatggcaagggtcagcaatacaacacaccgcagaagattgttggtattgcaggtgctgatattgccattgtcggacgtggaattatcaaggcgagtgaccccgaggaggaggctgatcgataccgatccgcagcgtggaaggcttacacagaacgcgttcgttga

SEQ ID NO: 52 - protein, 365 aa, Fusarium venenatum
MSSHPSLKATFASRAETASHPLSRHLYKLMDLKASNLCLSADVATARELLYFADKIGPSIVVLKTHYDMVAGWDFDPRTGTGAKLASLARKHGFLIFEDRKFGDIGNTVELQYTSGAARIIEWAHIVNVNMVPGKASVTSLAHAANRWLERYHYEVKTSISIGTPTASQLDEDSERSDGENQKSAPELGRDNGRKGSIVSTTTVTQQYESADSPRLVKTIPEGDETVFAGIDEAPIERGLLILAQMSSEGNFMNKEYTQACVEAAREHKSFVMGFISQECLNTQPDDDFIHMTPGCQLPPEGADENEAIKGDGKGQQYNTPQKIVGIAGADIAIVGRGIIKASDPEEEADRYRSAAWKAYTERVR

SEQ ID NO: 53 - DNA, 39 bp, Fusarium venenatum
aaaaaacctgcaggatcctgcgcggactcttgattattt

SEQ ID NO: 54 - DNA, 47 bp, Fusarium venenatum
aaaaaacctgcagggcggccgcaattccattcctgtagctgagtata

SEQ ID NO: 55 - DNA, 46 bp, Fusarium venenatum
aaaaaagtttaaacgcggccgcctgttgcctttgggccaatcaatg

SEQ ID NO: 56 - DNA, 38 bp, Fusarium venenatum
aaaaaagtttaaacctagttggagtattgtttgttctt

SEQ ID NO: 57 - DNA, 25 bp, Fusarium venenatum
ggatcatcatgacagcgtccgcaac

SEQ ID NO: 58 - DNA, 25 bp, Fusarium venenatum
ggcatagaaatctgcagcgctctct

SEQ ID NO: 59 - DNA, 1555 bp, Fusarium venenatum
atgaaacttcttcaacttgccaccctggtggcttccatcagcccattcgccagcgcagcagacgcaaacgcctggaagtcgcgaaacatttattttgctctcacagatcgtgttgcgcgtagcggtagtgatagcggcggtaacgcctgcggcaatctcggaaactattgcggtggaacctttaagggtcttgaggctaagctcgactacatcaagggcatgggattcgacgccatctggatcactcctgttgttgagagtaagtatcttggtcgtatatctgtttggatgatgtctaaccttttttgacagacacggatggcggataccacggatattgggccaaaaatctttacgaggtcaatgccaagtacggaaccaaagacgacctgaagagtctagtcaacactgcccatagcaaggtaagggggacatgatcctgcctgcacttcggcctttatctctgaagaaacttactgaccgccccagaacatgtacgtcatggctgacgtagtagcaaatcacatgggtccaggcatccaaaaccacagacccgaacctctgaaccaacaaagttcttaccactcttcctgcgcaatcgactacaacaaccaaaacagtatcgagcagtgtgagatcgctggcttgcccgatctcaacactggtagcgcaacagtcaagaaggttctcaacgactggatctcatggctcgtctccgaatacagcttcgatggtatccgcattgacaccgtcaagcacgtcgaaaagggcttctggcctgatttccagaaggccgctggagtcttctctatcggtgaagtctgggatggaagccctgattaccttgcagggtactcaaaggtcatgcctggtctattgaactacgccatctactaccccatgaaccgcttctaccagcagaagggtgacccatccgcagtggttgatatgtacaacgagatcagccaaaagtttgacgacccaactgtcctgggtaagtaattatgaagatgaggtataaatgcattaactaagtcgttacacaggaacattcatcgacaaccacgataatcctcgatggttaagccagaagaacgaccaggccctcctcaagaacgcccttgcctacgttattctctctcgtggtattcccattgtctattatggcaccgagcagggttacgctggaggcaatgaccccgcaaaccgtgaggatctctggcgtagcaacttcaagacagactcagacctttaccagactatctccaagctcggaaaggcccgctccgctgttggtggtctcgcaggaaacgaccagaagttcctcaagtccaatgacagtgcacttatctggagccgtgccgatagcgatctaattgttgtgactctgaatcgaggaaaaggattttccggagagtactgcttcaacactggcaagaacaacaagacttgggacagagtgctaggccaaggaactgtcaagtctgacggtaacggccagctatgtgttagctacactaacggtgaacccgaggttctcgttgcggcaaactaa

SEQ ID NO: 60 - protein, 460 aa, Fusarium venenatum
MKLLQLATLVASISPFASAADANAWKSRNIYFALTDRVARSGSDSGGNACGNLGNYCGGTFKGLEAKLDYIKGMGFDAIWITPVVENTDGGYHGYWAKNLYEVNAKYGTKDDLKSLVNTAHSKNMYVMADVVANHMGPGIQNHRPEPLNQQSSYHSSCAIDYNNQNSIEQCEIAGLPDLNTGSATVKKVLNDWISWLVSEYSFDGIRIDTVKHVEKGFWPDFQKAAGVFSIGEVWDGSPDYLAGYSKVMPGLLNYAIYYPMNRFYQQKGDPSAVVDMYNEISQKFDDPTVLGTFIDNHDNPRWLSQKNDQALLKNALAYVILSRGIPIVYYGTEQGYAGGNDPANREDLWRSNFKTDSDLYQTISKLGKARSAVGGLAGNDQKFLKSNDSALIWSRADSDLIVVTLNRGKGFSGEYCFNTGKNNKTWDRVLGPGTVKSDGNGQLCVSYTNGQPEVLVAAN

SEQ ID NO: 61 - DNA, 30 bp, Fusarium venenatum
gaggaattggatttggatgtgtgtggaata

SEQ ID NO: 62 - DNA, 28 bp, Fusarium venenatum
ggagtctttgttccaatgtgctcgttga

SEQ ID NO: 63 - DNA, 27 bp, Fusarium venenatum
ctacactaacggtgaacccgaggttct

SEQ ID NO: 64 - DNA, 27 bp, Fusarium venenatum
gcggcaaactaatgggtggtcgagttt

SEQ ID NO: 65 - DNA, 39 bp, Fusarium venenatum
aaaaaacctgcaggtaatgggtggtcgagtttaaaagta

SEQ ID NO: 66 - DNA, 48 bp, Fusarium venenatum
aaaaaacctgcagggcggccgctttaagcatcatttttgactacgcac

SEQ ID NO: 67 - DNA, 52 bp, Fusarium venenatum
aaaaaagtttaaacgcggccgcttgattatgggatgaccccagacaagtggt

SEQ ID NO: 68 - DNA, 43 bp, Fusarium venenatum
aaaaaagtttaaacccgcacgagcgtgtttccttttcatctcg

SEQ ID NO: 69 - DNA, 25 bp, Fusarium venenatum
ggatcatcatgacagcgtccgcaac

SEQ ID NO: 70 - DNA, 25 bp, Fusarium venenatum
ggcatagaaatctgcagcgctctct

SEQ ID NO: 71 - DNA, 1488 bp, Microdochium nivale
atgcgttctgcatttatcttggccctcggccttatcaccgccagcgctgacgctttagtcactcgcggtgccatcgaggcctgcctgtctgctgctggcgtcccgatcgatattcctggcactgccgactatgagcgcgatgtcgagcccttcaacatccgcctgccatacattcccaccgccattgctcagacgcagactactgctcacatccagtcggcagtccagtgcgccaagaagctcaacctcaaggtctctgccaagtctggtggtcacagctacgcctcgttcggctttggtggcgagaacggtcacctcatggtccagctcgaccgcatgattgatgtcatctcgtacaatgacaagactggcattgcccatgttgagcccggtgcccgcctcggacatctcgccaccgtcctcaacgacaagtacggccgtgccatctcccacggtacatgccctggtgtcggcatctccggccactttgcccacggcggcttcggcttcagctcgcacatgcacggtctggctgtcgactcggtcgtcggtgtcactgttgttcttgctgatggacgcatcgttgaggcttctgccactgagaatgctgacctcttctggggtatcaagggcgctggctccaacttcggcatcgttgctgtctggaagctcgccactttccctgctcccaaggttctcacccgctttggcgtcaccctcaactggaagaacaagacctctgccctcaagggcatcgaggctgttgaggactacgcccgctgggtcgccccccgcgaggtcaacttccgcattggagactacggcgctggtaacccgggtatcgagggtctctactacggcactcccgagcaatggcgtgcggctttccaacctctgctcgacactctgcctgctggatacgttgtcaacccgaccacctccttgaactggatcgagtcggtgctcagctactccaactttgaccatgtcgacttcattactcctcagcctgtcgagaacttctatgccaagagcttgacgctcaagagtatcaagggcgacgccgtcaagaactttgtcgactactactttgacgtgtccaacaaggttaaggaccgcttctggttctaccagctcgacgtgcacggcggcaagaactcgcaagtcaccaaggtcaccaacgccgagacagcctaccctcaccgcgacaagctctggctgatccagttctacgaccgctacgacaacaaccagacctacccggagacctcattcaagttcctcgacggctgggtcaactcggtcaccaaggctctccccaagtccgactggggcatgtacatcaactacgccgacccccgcatggaccgcgactacgccaccaaggtctactacggtgagaacctcgccaggctccagaagctcaaggccaagtttgatcccaccgaccgtttctactaccctcaggctgtccgccctgtcaaataa

SEQ ID NO: 72 - protein, 495 aa, Microdochium nivale
MRSAFILALGLITASADALVTRGAIEACLSAAGVPIDIPGTADYERDVEPFNIRLPYIPTAIAQTQTTAHIQSAVQCAKKLNLKVSAKSGGHSYASFGFGGENGHLMVQLDRMIDVISYNDKTGIAHVEPGARLGHLATVLNDKYGRAISHGTCPGVGISGHFAHGGFGFSSHMHGLAVDSVVGVTVVLADGRIVEASATENADLFWGIKGAGSNFGIVAVWKLATFPAPKVLTRFGVTLNWKNKTSALKGIEAVEDYARWVAPREVNFRIGDYGAGNPGIEGLYYGTPEQWRAAFQPLLDTLPAGYVVNPTTSLNWIESVLSYSNFDHVDFITPQPVENFYAKSLTLKSIKGDAVKNFVDYYFDVSNKVKDRFWFYQLDVHGGKNSQVTKVTNAETAYPHRDKLWLIQFYDRYDNNQTYPETSFKFLDGWVNSVTKALPKSDWGMYINYADPRMDRDYATKVYYGENLARLQKLKAKFDPTDRFYYPQAVRPVK

SEQ ID NO: 73 - DNA, 26 bp, Microdochium nivale
cccgcatgcgttctgcatttatcttg

SEQ ID NO: 74 - DNA, 26 bp, Microdochium nivale
gggttaattaattatttgacagggcg

SEQ ID NO: 75 - DNA, 1389 bp, Candida antarctica
atgcgagtgtccttgcgctccatcacgtcgctgcttgcggcggcaacggcggctgtgctcgcggctccggcggccgagacgctggaccgacgggcggcgctgcccaacccctacgacgatcccttctacacgacgccatccaacatcggcacgtttgccaagggccaggtgatccaatctcgcaaggtgcccacggacatcggcaacgccaacaacgctgcgtcgttccagctgcagtaccgcaccaccaatacgcagaacgaggcggtggccgacgtggccaccgtgtggatcccggccaagcccgcttcgccgcccaagatcttttcgtaccaggtctacgaggatgccacggcgctcgactgtgctccgagctacagctacctcactggattggaccagccgaacaaggtgacggcggtgctcgacacgcccatcatcatcggctgggcgctgcagcagggctactacgtcgtctcgtccgaccacgaaggcttcaaagccgccttcatcgctggctacgaagagggcatggctatcctcgacggcatccgcgcgctcaagaactaccagaacctgccatccgacagcaaggtcgctcttgagggctacagtggcggagctcacgccaccgtgtgggcgacttcgcttgctgaatcgtacgcgcccgagctcaacattgtcggtgcttcgcacggcggcacgcccgtgagcgccaaggacacctttacattcctcaacggcggacccttcgccggctttgccctggcgggtgtttcgggtctctcgctcgctcatcctgatatggagagcttcattgaggcccgattgaacgccaagggtcagcggacgctcaagcagatccgcggccgtggcttctgcctgccgcaggtggtgttgacctaccccttcctcaacgtcttctcgctggtcaacgacacgaacctgctgaatgaggcgccgatcgctagcatcctcaagcaggagactgtggtccaggccgaagcgagctacacggtatcggtgcccaagttcccgcgcttcatctggcatgcgatccccgacgagatcgtgccgtaccagcctgcggctacctacgtcaaggagcaatgtgccaagggcgccaacatcaatttttcgccctacccgatcgccgagcacctcaccgccgagatctttggtctggtgcctagcctgtggtttatcaagcaagccttcgacggcaccacacccaaggtgatctgcggcactcccatccctgctatcgctggcatcaccacgccctcggcggaccaagtgctgggttcggacctggccaaccagctgcgcagcctcgacggcaagcagagtgcgttcggcaagccctttggccccatcacaccaccttag

SEQ ID NO: 76 - protein, 462 aa, Candida antarctica
MRVSLRSITSLLAAATAAVLAAPAAETLDRRAALPNPYDDPFYTTPSNIGTFAKGQVIQSRKVPTDIGNANNAASFQLQYRTTNTQNEAVADVATVWIPAKPASPPKIFSYQVYEDATALDCAPSYSYLTGLDQPNKVTAVLDTPIIIGWALQQGYYVVSSDHEGFKAAFIAGYEEGMAILDGIRALKNYQNLPSDSKVALEGYSGGAHATVWATSLAESYAPELNIVGASHGGTPVSAKDTFTFLNGGPFAGFALAGVSGLSLAHPDMESFIEARLNAKGQRTLKQIRGRGFCLPQVVLTYPFLNVFSLVNDTNLLNEAPIASILKQETVVQAEASYTVSVPKFPRFIWHAIPDEIVPYQPAATYVKEQCAKGANINFSPYPIAEHLTAEIFGLVPSLWFIKQAFDGTTPKVICGTPIPAIAGITTPSADQVLGSDLANQLRSLDGKQSAFGKPFGPITPP

SEQ ID NO: 77 - DNA, 20 bp, Candida antarctica
gcatgcgagtgtccttgcgc

SEQ ID NO: 78 - DNA, 24 bp, Candida antarctica
ttaattaactaaggtggtgtgatg

SEQ ID NO: 79 - DNA, 31 bp, Fusarium oxysporum
tcagatttaaatatgcttcttctaccactcc

SEQ ID NO: 80 - DNA, 28 bp, Fusarium oxysporum
agtcttaattaaagctagtgaatgaaat

SEQ ID NO: 81 - DNA, 27 bp, Fusarium venenatum
gcaggaaagaacaagtgagcaaaaggc

SEQ ID NO: 82 - DNA, 27 bp, Fusarium venenatum
gccttttgctcacttgttctttcctgc

SEQ ID NO: 83 - DNA, 15 bp, Fusarium oxysporum
dcctacatgtttaat

SEQ ID NO: 84 - DNA, 13 bp, Fusarium oxysporum
dtaaacatgtagg

SEQ ID NO: 85 - DNA, 25 bp, Fusarium oxysporum
gggggcatgcttcttctaccactcc

SEQ ID NO: 86 - DNA, 27 bp, Fusarium oxysporum
ggggttaattaagagcgggcctggtta

SEQ ID NO: 87 - DNA, 45 bp, Fusarium venenatum
cctgcatggccgccgccgccaattcttacaaaccttcaacagtgg

SEQ ID NO: 88 - DNA, 45 bp, Fusarium venenatum
ccactgttgaaggtttgtaagaattggcggcggcggccatgcagg

SEQ ID NO: 89 - DNA, 35 bp, Fusarium venenatum
ataagaatgcggccgctccaaggaatagaatcact

SEQ ID NO: 90 - DNA, 25 bp, Fusarium venenatum
cggaattctgtcgtcgaatactaac

SEQ ID NO: 91 - DNA, 1289 bp, Fusarium venenatum
atgaccagcttccgccgtctcgctctctgccttggagctctgctccctgcagtcctcgccgctcccactgagaagcgacaggaactcactgccgcacctgacaagtacatcatcactctcaagcccgaggcttctgagaccaaggttgaggctcacttgaactgggtcagcgatgttcaccgtcgcagccttaacaagcgtgatacctctggtgtcgagaagaagttcaacatcagcacctggaacgcctactctggcgagttcgacaaggctaccattgatgagatcaagaagagccccgaggttgctttcgtcgagcctgactacactgtctacctcgactacgagaccgagctgtctgaccgtgccttgactacccagagtggtgctccttggggtcttgcctccatctcccgccgaacctccggtggcagcacttacacctacgacaccactgccggctccggtgcttacggttacgtcgttgacagcggcatcaacgtcgaccaccgagatttcggtggccgtgcttctctcggcttcaacgctgccggtggtgctcacgtcgacacccttggccacggtacccacgtcgctggaaccattgcttctgccacctacggtgttgccaagcgcgtaagtaaaccccacaatttatggtagcatctgaactttatacttactatctttaggccaacgtcatctctgtcaaggtcttcaccggtaacagtggttccacctccactatcctctctggtttcaactgggctgtcaacgacatcacctccaagggacgcaccggccgctctgtcatcaacctgtctctcggtggtcccgcttctcagacctggaccactgctatcaacgctgcttacaactctggtgttctctccgttgttgctgccggtaacggtgacgatttcggccgccctcttcccgtctctggccagtctcctgccaacgtccccaacgctctgaccgttgctgccattgactccagctggcgcactgcctctttcaccaactacggtgccggtgttgatgtcttcgccctggtgtcagcatcctctcactggattggttccacctctgctaccaactccatcagcggtacctccatggcctgccctcacgttgctggtcttgctctctacctccaggttctcgagggtctttccacccctgctgctgttaccaaccgcatcaaggctcttgctaccactggccgtgtcactggcaccctgagcggtagccccaacctgatcgccttcaacggtgcttccgcttaa

SEQ ID NO: 92 - protein, 410 aa, Fusarium venenatum
MTSFRRLALCLGALLPAVLAAPTEKRQELTAAPDKYIITLKPEASETKVEAHLNWVSDVHRRSLNKRDTSGVEKKFNISTWNAYSGEFDKATIDEIKKSPEVAFVEPDYTVYLDYETELSDRALTTQSGAPWGLASISRRTSGGSTYTYDTTAGSGAYGYVVDSGINVDHRDFGGRASLGFNAAGGAHVDTLGHGTHVAGTIASATYGVAKRANVISVKVFTGNSGSTSTILSGFNWAVNDITSKGRTGRSVINLSLGGPASQTWTTAINAAYNSGVLSVVAAGNGDDFGRPLPVSGQSPANVPNALTVAAIDSSWRTASFTNYGAGVDVFALVSASSHWIGSTSATNSISGTSMACPHVAGLALYLQVLEGLSTPAAVTNRIKALATTGRVTGTLSGSPNLIAFNGASA

SEQ ID NO: 93 - DNA, 30 bp, Fusarium venenatum; identical to SEQ ID NO: 61

SEQ ID NO: 94 - DNA, 28 bp, Fusarium venenatum
ggagtctttgttccaatgtgctcgttga

SEQ ID NO: 95 - DNA, 48 bp, Fusarium venenatum
aaaaaaggcgcgccgcggccgcgttacggtgttcaagtacatcttaca

SEQ ID NO: 96 - DNA, 39 bp, Fusarium venenatum
aaaaaaggcgcgccattgctatcatcaactgcctttctt

SEQ ID NO: 97 - DNA, 36 bp, Fusarium venenatum
aaaaacctgcaggggatgtgtgtggaataggatatg

SEQ ID NO: 98 - DNA, 45 bp, Fusarium venenatum
aaaaacctgcagggcggccgccctcaaggtggagaaataatctgt

SEQ ID NO: 99 - DNA, 25 bp, Fusarium venenatum
gcacgttaggctcaagccagcaagg

SEQ ID NO: 100 - DNA, 25 bp, Fusarium venenatum
gaggctcatggatgtggcgttaatg

SEQ ID NO: 101 - DNA, 9390 bp, Fusarium venenatum
atggaatatcttactgctgtcgatggtaggcaagacctgccacctacaccagcttcgttttgtagtcatggagatagtcccctcaatagctcttacgagcaactcttccatctctatggtctggattcgagtcgcatcgaagctatcaaaccatgcacacctttccagcttgacatgatcgactgcaatgctttggataagcagtctgctatcggccatgcggtgtatgatgtcccaaccgacattgacatctctcgtttcgcgcttgcgtggaaggagatcgtcaaccaaaccccagccttgcgagcctttgccttcacctcggactctggaaagacttctcaagtcatcctaaaagatagctttgtcttctcatggatgtgctggtcttcttcgagctccccagatgaagtggttcgggatgaagctgccgctgctgcatccgggccacgctgcaaccgcttcgttctacttgaagacatgcagacgaagaaatgtcagctggtttggaccttcagtcatgcattggtagacgtcactttccaacaacgcgtcctgagccgtgttttcgcggcttacaagcatgagaaggacacacatcggcctgagacacccgagtcatctgatgccactgacactgactctcagtcagtctccgtggtgtccatgagctgcgaggacaatgccgtatcggcgactcatttctggcaaactcaccttaacgatctcaatgcgtccgtcttccctcacctgtctgaccacctgatggtgcccaacccaactacaacagcagagcatcgtatcacattccctctttcacagaaagcactatccaattctgccatctgccgtactgcactctcaatactcctctcgcgctacactcactctgacgaggccttgtttggtgcggtaactgagcaatctctaccatttgacaaacactatcttgcagatggtacgtaccaaacagttgcaccccttcgtgtacactgccaatcaaatcttcgtgcatcagatgtcatggatgcaatctcttcttacgatgatcgccttggtcatctcgccccatttggccttcgcgacatccgcaacactggtgataatggctctgccgcctgcgatttccaaactgttttactcgtcaccgatggcagccacgtaaacaatggtatcaacggtttcctccaacagataacagagtcaagccatttcatgccttgcaacaaccgtgccctccttctgcactgtcagatggaaagtagcggagctctgctggttgcctactatgaccacaatgttatcgattcgcttcagacaacgcgtctgctacagcagtttggtcatctgatcaagtgtttgcaaagtcctctagacctgagctctatggctgaggtcaacttgatgactgagtatgacagagcagagattgagagttggaactcgcaaccgttagaggtacaggataccctgatccaccatgagatgttgaaagctgtttctcattcccccaccaaaacggccatccaagcgtgggatggagactggacctattccgagctcgacaatgtttcgtcaagactcgctgtccatatcaagtcacttggccttagagctcagcaagccattattccagtctactttgagaagtcgaaatgggtcattgcttcaatgctggctgttctcaagtctggtaatgctttcactctaattgatcccaatgatccaccagctcgaactgcccaggtcgtcacgcagactcgggcgactgtagcgcttacttccaagctacaccgcgagactgtacagaagcttgtaggccgttgcgttgtggttgatgacgagcttctgcaatcagtttctgccagcgacgatttctcaagtctgaccaaatcgcaagacttggcctacgtgatcttcacttctggtagcacgggcgacccgaaaggcatcatgattgaacaccgagcgttctcatcatgtgcactcaagttcggcgcgtctcttggcatcaactctgatactcgtgccctacaatttggaacccatgcctttggcgcatgtcttctcgagattatgactactctcatcaacggtggctgcgtttgtattccctccgacgatgatcgtatgaacagtatcccgtccttcatcaaccgatacaacgttaattggatgatggcgacaccttcgtacatgggaaccttttcacctgaagacgttcctggccttgcgacattggtacttgttggggagcagatgtcatcttcagtcaacgcaatctgggcccccaagctccaactcttgaacgggtacggacagagtgaaagttcctcaatttgttttgcctccaatatgtcaactgagcccaacaacatgggcagagcagtcggagctcattcatgggtcattgacccgaacgatataaaccgactagttccgattggagctgtgggagaactggtcattgagagtccaggcattgcccgcgactacattgttcccccccctccggagaagtccccattcttcacagacattccaagctggtatccagcgaacacgtttcctgatggggcaaaactctacaggacaggagatcttgcaagatatgcctccgatgggtccatcgtttgccttgggcgcatagactcgcaggtcaagatccggggacagcgtgttgagctgggtgccattgagacccatctccgacagcagatgccagacgacttgactattgtggtagaagctaccaagcgatcccaatctgccaacagcacatccttaattgcattcctaatagggtcttcttacttcggaaatagaccctcggatgcccacattctggaccatgatgctaccaaagctatcaacataaagctggagcaggtattgcctcgacactctatcccctcattctacatctgcatgctggagcttccacgtactgccaccgggaagatagataggaggcgactacgaatcatgggcaaagacatcttggacaagcagacccaaggggccattgttcaacaagcacccgctcctatccctgttttcgcagacacagcagcaaagctccacagtatctgggtacagagtttgggtatcgatccagccacggtcaatgttggggcaactttcttcgaactcggaggaaactctatcactgctatcaagatggtgaacatggcgaggtccgttggtatggacctcaaggtctctaacatctaccagcacccgacgcttgcgggaatttccgcggtcgtcaagggtgatcctctgtcctacactctcatccccaagtcaactcatgagggacctgttgagcagtcttattcacaaggccgactatggttcctggatcagttggacgttggcagtctgtggtatctgattccatatgctgtgagaatgcgcgggcctgtcaatgtcgacgcgttacgtcgggctcttgcagcgcttgaacagcgacacgagactcttagaacgacatttgaagaccaggatggtgtcggtgtacaaattgttcacgagaagctttctgaggagatgaaggtcattgatctctgtggttcagaccttgacccgtttgaggtgttgaaccaagaacagactactcccttcaatctctcatctgaagctggctggagagcgacgctcttacgacttggtgaagatgaccacatcctcactattgtcatgcatcacatcatctcagatggttggtcaattgatgtcttgcgacgcgatctcaatcagctctactcagctgcgctcaaggactcaaaagacccgctgtcagcactcactcctctacctatccagtacagcgactttgcaaaatggcagaaggaccaattcatagagcaggagaagcaactcaactactggaagaagcaactcaaagactcttccccagcaaagatcccgaccgactttgcccgccctgcacttctgtctggagacgcaggttgcgtacatgttaccatcgacggcgagctctaccagtcccttcgagccttctgcaacgaacacaacacgacctctttcgtcgttcttctagctgcgttccgtgccgctcattatcgtctcacagctgttgaagacgctgtcattggtacaccaattgcgaatcgcaaccgacctgaactggaggatatcatcggctgctttgtcaatacgcagtgtatgcgaatcaacatagatcatcacgatacctttgggactttgatcaaccaagtcaaggctacgacgacagcagcattcgagaacgaggatattccgtttgagcgcgttgtatcagcactacagcctggatccagagatctgtcaagcacacctctcgcacaactcatttttgcagtgcactcacagaaggaccttggaagattcaagttccagggtctcgagtccgtacctgtgcctagcaaagcgtacactcgatttgacatggagttccatctgtttcaagaaaccgacagccttaaaggtagcgtcaactttgccgatgagctgttcaaaatggagactgttgaaaatgtcgtcagagtattctttgagattctgagaaacgggcttcaaagttcgcggacaccagtctcaatacttcctttgactgatggcattgtgactcttgaaaaattggatgttctcaacgtcaaacatgtcgactatccccgagaatcgagcttggctgatgtcttccagacccaagtctctgcttaccccgatagtctggctgtggtggactcctcgtgccgattgacctacaccgagttggatcgccagtctgatattctcgctggatggcttcgtcgacggtcaatgcctgcagagacgcttgtcgcagtatttgccccacggtcatgtgagacaattgtcgcgttctttggtgtgttgaaggcgaacttggcctatcttcctctcgatgtacgatcgccctcggcgagagttcaggatatactttctggactttctgggcctaccattgttttgattggccatgatacagcgcctcccgatatcgaggttactaacgtcgagtttgttcgtatccgggatgcgctgaatgacagcaatgcagatggctttgaagtcatcgagcacgacagcacaaagccctcagccacgagtctcgcatacgtgctgtatacctcaggatccactggccgaccaaaaggcgtcatgattgagcaccgtgtcattattcgaacagtcacaagtggctgtatacccaactatccttcggaaacgaggatggctcacatggcgaccattgcgtttgacggcgcatcgtacgagatctacagcgcccttttgttcggaaggacacttgtttgcgttgactacatgacaaccctcgacgctagagcactcaaggatgtgtttttccgagagcatgtcaacgcggcaagtcatgtcaccagctcttctcaagatgtacctctccgagtcccgagaaggctctcgagaaccttgatgttcttcttcttggtggtgacagattcgacggccccagatgctctcgatgcgcagggactttatcaaggggtccagtgttacaatggttacggcccaacagagaatggagtcatgagtacaatctatcccattgactcgactgagtcgttcatcaatggagtcccaattggacgagctctgaacaactcaggagcgtatgtcgtggatcctgagcaacagcttgttggcattggtgtgatgggagagcttgttgtcactggcgatggtcttgcgcggggctacagtgacaaagcccttgacgagaaccgttttgtgcacattactgtcaatgaccagacagtgaaggcgtatcgcactggcgatcgagtgcggtacaggattggagatggcctcatcgagttcttcggacgtatggacacccagttcaagattcgtggcaatcgtatcgaatcagctgagattgaagcggcccttctgcgcgactcctccgtccgagatgctgctgtcgtccttcagcagaatgaggatcaagcgcctgagatcttggggtttgttgttgctgatcatgatcattctgagaatgacaagggacaatctgccaatcaagtcgaaggatggcaagaccatttcgagagtggcatgtattccgacattggcgaaattgacccgtcgacgattggtagcgacttcaagggttggacatcaatgtatgatggaagtcaaatcgacttcgatgagatgcacgagtggcttggtgagactacccggacactccatgacaatcgctctctaggcaatgtccttgaaattggaacaggtagcggcatgatcctcttcaaccttgacagcaggcttgagagttacgttggtcttgaaccatccagatcagcagctgcatttgtcaacaaagctaccgagtctataccatcgcttgctggaaaagccaaggttcaggttggaacagctacagatattggtcaagtcgatgacttacaccctgacctcgtggttctcaactcagtcattcagtatttcccgtcttcggagtaccttgcagaaatcgcagacaccttgattcatctgcctaacgtgcagcggattttctttggcgatgtccgatcgcaggccaccaacgagcacttccttgctgccagggctatccacacactggggaagaatgcaacgaaggacgatgttcgacagaaaatggcagaattggaggacatggaggaggagttgcttgttgaacctgctttcttcacctcgttgaaagacaggtttccaggtctggtggaacatgttgagatcctgccaaagaacatggaagctgtgaatgagctcagtgcgtatcgatatgccgctgttgtgcacgttcggggttcacttggagatgagcttgtgcttccggttgagaaagatgactggatcgactttcaagcgaatcaattgaaccagaagtcactgggtgaccttctcaagtcttcagatgctgctatcatggcagtcagcaaaattcctttcgaaatcacggcctttgaaagacaggtcgtcgcttccctcaatagcaacatcgatgagtggcagctatcaaccattcggtccagcgccgagggcgactcatcactatccgttcccgacatctttcgcattgctggggaagccgggttccgtgtcgaggtcagttctgcacgacagtggtctcagaatggtgcattggacgctgttttccatcattgttgctcccaagggcgtactctggtcaactttcctacggaccatcaccttcgagggtctgatctcctcaccaatcgaccccttcagcgactgcaaaaccgtcgtatcgccatcgaagtccgcgagaggcttcggtccttacttccatcgtacatgatcccatcgaacatcgttgttctggacaagatgcctctcaacgccaatggtaaagttgaccggaaggaactctctcgcagggcaaaggttgtaccgaagcagcagacagcagcgccgttaccgacatttcccatcagtgaggtcgaagtcattctttgcgaagaagccactgaggtgtttggcatgaaggttgacattaccgatcacttcttcaatctcggtggacactctctcttggccacgaagctcatttctcgtatcgaccaacgactcaaggtccgtatcactgtcaaggatgtctttgaccatcctgtatttgcggatctagcatctgtcatccgtcaagggctgggtttgcaacaacccgtttctgatggtcagggacaagacagatctgcccacatggcaccccgtaccgagactgaagctatactctgtgatgagtttgcaaaggttctggggttccaagtcgggattacagacaatttctttgatcttggtggtcattcactcatggctactaaactcgctgtgcgcatcggacatcgacttgacacgactgtttcggtgaaggatgttttcgatcatcctgtactcttccaacttgcaattgcattggataacttggttcaatccaagaccaatgagatagttggaggtagagaaatggctgaatactcacctttccaactcctctttacagaagacccagaggagtttatggcgagcgagatcaagccacaacttgagttacaggaaatcattcaagacatatatccgtctacccagatgcagaaggctttcctcttcgatcacacaactgcgcgcccgagacctttcgtgccgttctacatcgacttccccagcacttccgagcctgatgctgcaggtctaatcaaggcttgcgagtctctggtaaatcatcttgacatcttcagaacagtctttgcagaggcatctggagaactataccaagtggtcttgtcctgtcttgatctgccaatccaagtgattgagacagaagacaacatcaatacggcgacaaatgagtttctcgatgagtttgcgaaagagccagttcgtctgggacatccgttgattcgttttacaatcatcaaacaaaccaagtcgatgcgtgtgataatgagaatatcgcatgccctgtatgatggtctgagtctagagcatgtcgtgcgcaaacttcacatgctctacaacgggagatcacttttgccaccacaccaattctcgcggtacatgcagtatactgctgacggtcgcgaaagtggacatggattttggcgcgatgtgattcaaaatacgcccatgacaatattgagtgatgacacggttgttgatggaaatgatgcaacctgcaaggcgttgcacctatcaaagattgtcaatattccttcacaggtacttcgaggcagcagtaacatcattactcaagctactgtgtttaacgcagcctgcgcgttagtcttgtcacgggaatctgactcgaaagacgttgtctttggacgcatcgtctctggtcgtcaaggcttgcctgttgaataccaggacattgtcgggccttgtaccaacgcagttcctgttcgcgctcatatagagtcgtcagattacaaccaattgctgcacgacatccaagaccagtaccttctcagcttgccacacgaaacaattggcttctcagatctcaagcgcaactgtacagattggccagaagcaatcaccaacttctcatgctgcatcacataccacaatttcgagtaccatcccgagagtcagttcgaacagcagagagttgagatgggtgtattgacaaagtttgtcaacattgagatggatgagccactatatgatttggcgattgcgggtgaagttgaaccagacggagcaggactgaaggttactgttatcgcgaagacgcagttatttggtaggaagagagtagaacatctgttggaggaagtttccaaaacgtttgagggtctcaactcttctttgtaa

SEQ ID NO: 102 - protein, 3129 aa, Fusarium venenatum
MEYLTAVDGRQDLPPTPASFCSHGDSPLNSSYEQLFHLYGLDSSRIEAIKPCTPFQLDMIDCNALDKQSAIGHAVYDVPTDIDISRFALAWKEIVNQTPALRAFAFTSDSGKTSQVILKDSFVFSWMCWSSSSSPDEVVRDEAAAAASGPRCNRFVLLEDMQTKKCQLVWTFSHALVDVTFQQRVLSRVFAAYKHEKDTHRPETPESSDATDTDSQSVSVVSMSCEDNAVSATHFWQTHLNDLNASVFPHLSDHLMVPNPTTTAEHRITFPLSQKALSNSAICRTALSILLSRYTHSDEALFGAVTEQSLPFDKHYLADGTYQTVAPLRVHCQSNLRASDVMDAISSYDDRLGHLAPFGLRDIRNTGDNGSAACDFQTVLLVTDGSHVNNGINGFLQQITESSHFMPCNNRALLLHCQMESSGALLVAYYDHNVIDSLQTTRLLQQFGHLIKCLQSPLDLSSMAEVNLMTEYDRAEIESWNSQPLEVQDTLIHHEMLKAVSHSPTKTAIQAWDGDWTYSELDNVSSRLAVHIKSLGLRAQQAIIPVYFEKSKWVIASMLAVLKSGNAFTLIDPNDPPARTAQVVTQTRATVALTSKLHRETVQKLVGRCVVVDDELLQSVSASDDFSSLTKSQDLAYVIFTSGSTGDPKGIMIEHRAFSSCALKFGASLGINSDTRALQFGTHAFGACLLEIMTTLINGGCVCIPSDDDRMNSIPSFINRYNVNWMMATPSYMGTFSPEDVPGLATLVLVGEQMSSSVNAIWAPKLQLLNGYGQSESSSICFASNMSTEPNNMGRAVGAHSWVIDPNDINRLVPIGAVGELVIESPGIARDYIVPPPPEKSPFFTDIPSWYPANTFPDGAKLYRTGDLARYASDGSIVCLGRIDSQVKIRGQRVELGAIETHLRQQMPDDLTIVVEATKRSQSANSTSLIAFLIGSSYFGNRPSDAHILDHDATKAINIKLEQVLPRHSIPSFYICMLELPRTATGKIDRRRLRIMGKDILDKQTQGAIVQQAPAPIPVFADTAAKLHSIWVQSLGIDPATVNVGATFFELGGNSITAIKMVNMARSVGMDLKVSNIYQHPTLAGISAVVKGDPLSYTLIPKSTHEGPVEQSYSQGRLWFLDQLDVGSLWYLIPYAVRMRGPVNVDALRRALAALEQRHETLRTTFEDQDGVGVQIVHEKLSEEMKVIDLCGSDLDPFEVLNQEQTTPFNLSSEAGWRATLLRLGEDDHILTIVMHHIISDGWSIDVLRRDLNQLYSAALKDSKDPLSALTPLPIQYSDFAKWQKDQFIEQEKQLNYWKKQLKDSSPAKIPTDFARPALLSGDAGCVHVTIDGELYQSLRAFCNEHNTTSFVVLLAAFRAAHYRLTAVEDAVIGTPIANRNRPELEDIIGCFVNTQCMRINIDHHDTFGTLINQVKATTTAAFENEDIPFERVVSALQPGSRDLSSTPLAQLIFAVHSQKDLGRFKFQGLESVPVPSKAYTRFDMEFHLFQETDSLKGSVNFADELFKMETVENVVRVFFEILRNGLQSSRTPVSILPLTDGIVTLEKLDVLNVKHVDYPRESSLADVFQTQVSAYPDSLAVVDSSCRLTYTELDRQSDILAGWLRRRSMPAETLVAVFAPRSCETIVAFFGVLKANLAYLPLDVRSPSARVQDILSGLSGPTIVLIGHDTAPPDIEVTNVEFVRIRDALNDSNADGFEVIEHDSTKPSATSLAYVLYTSGSTGRPKGVMIEHRVIIRTVTSGCIPNYPSETRMAHMATIAFDGASYEIYSALLFGRTLVCVDYMTTLDARALKDVFFREHVNAASHVTSSSQDVPLRVPRRLSRTLMFFFLVVTDSTAPDALDAQGLYQGVQCYNGYGPTENGVMSTIYPIDSTESFINGVPIGRALNNSGAYVVDPEQQLVGIGVMGELVVTGDGLARGYSDKALDENRFVHITVNDQTVKAYRTGDRVRYRIGDGLIEFFGRMDTQFKIRGNRIESAEIEAALLRDSSVRDAAVVLQQNEDQAPEILGFVVADHDHSENDKGQSANQVEGWQDHFESGMYSDIGEIDPSTIGSDFKGWTSMYDGSQIDFDEMHEWLGETTRTLHDNRSLGNVLEIGTGSGMILFNLDSRLESYVGLEPSRSAAAFVNKATESIPSLAGKAKVQVGTATDIGQVDDLHPDLVVLNSVIQYFPSSEYLAEIADTLIHLPNVQRIFFGDVRSQATNEHFLAARAIHTLGKNATKDDVRQKMAELEDMEEELLVEPAFFTSLKDRFPGLVEHVEILPKNMEAVNELSAYRYAAVVHVRGSLGDELVLPVEKDDWIDFQANQLNQKSLGDLLKSSDAAIMAVSKIPFEITAFERQVVASLNSNIDEWQLSTIRSSAEGDSSLSVPDIFRIAGEAGFRVEVSSARQWSQNGALDAVFHHCCSQGRTLVNFPTDHHLRGSDLLTNRPLQRLQNRRIAIEVRERLRSLLPSYMIPSNIVVLDKMPLNANGKVDRKELSRRAKVVPKQQTAAPLPTFPISEVEVILCEEATEVFGMKVDITDHFFNLGGHSLLATKLISRIDQRLKVRITVKDVFDHPVFADLASVIRQGLGLQQPVSDGQGQDRSAHMAPRTETEAILCDEFAKVLGFQVGITDNFFDLGGHSLMATKLAVRIGHRLDTTVSVKDVFDHPVLFQLAIALDNLVQSKTNEIVGGREMAEYSPFQLLFTEDPEEFMASEIKPQLELQEIIQDIYPSTQMQKAFLFDHTTARPRPFVPFYIDFPSTSEPDAAGLIKACESLVNHLDIFRTVFAEASGELYQVVLSCLDLPIQVIETEDNINTATNEFLDEFAKEPVRLGHPLIRFTIIKQTKSMRVIMRISHALYDGLSLEHVVRKLHMLYNGRSLLPPHQFSRYMQYTADGRESGHGFWRDVIQNTPMTILSDDTVVDGNDATCKALHLSKIVNIPSQVLRGSSNIITQATVFNAACALVLSRESDSKDVVFGRIVSGRQGLPVEYQDIVGPCTNAVPVRAHIESSDYNQLLHDIQDQYLLSLPHETIGFSDLKRNCTDWPEAITNFSCCITYHNFEYHPESQFEQQRVEMGVLTKFVNIEMDEPLYDLAIAGEVEPDGAGLKVTVIAKTQLFGRKRVEHLLEEVSKTFEGLNSSL

SEQ ID NO: 103 - DNA, 38 bp, Fusarium venenatum
gactaagccctgcaggttggtctcaatcgtcgcgacag

SEQ ID NO: 104 - DNA, 46 bp, Fusarium venenatum
agtctacccctgcaggcggccgctggcatcggtggacgtaacacgc

SEQ ID NO: 105 - DNA, 30 bp, Fusarium venenatum
gctattgaggggactatctccatgactaca

SEQ ID NO: 106 - DNA, 29 bp, Fusarium venenatum
gcctaccatcgacagcagtaagatattcc

SEQ ID NO: 107 - DNA, 50 bp, Fusarium venenatum
atgtgctacaggcgcgccgcggccgcgagttccaacatgtcttattatcc

SEQ ID NO: 108 - DNA, 41 bp, Fusarium venenatum
tactgtaccggcgcgccatctgagccaagagactcattcat

SEQ ID NO: 109 - DNA, 25 bp, Fusarium venenatum
cttgactattatctcacgttgtcag

SEQ ID NO: 110 - DNA, 25 bp, Fusarium venenatum
tcaagtgttgtgtaatgttggaaca

SEQ ID NO: 111 - DNA, 42 bp, Trichoderma reesei
tatagcgtacctgcaggtgtcatgcccgcggctttgccttga

SEQ ID NO: 112 - DNA, 48 bp, Trichoderma reesei
atgctgtacctgcaggcggccgccgctcccgatcatcatccctccgag

SEQ ID NO: 113 - DNA, 56 bp, Trichoderma reesei
catggtttaaacggcggcgcgccgcggccgcaattcagagcatcacggttgaggga

SEQ ID NO: 114 - DNA, 45 bp, Trichoderma reesei
cttgttttgtcgggcgcgccacatggccttggattgacgcaggac

SEQ ID NO: 115 - DNA, 35 bp, Trichoderma reesei
gacgcatacaatacaagcatatgctgttggtgtct

SEQ ID NO: 116 - DNA, 25 bp, Trichoderma reesei
aaggcgtctggaaacagaagctgct